(19) United States
(12) Patent Application Publication
(10) Pub. No.: US 2021/0380976 A1
(43) Pub. Date: Dec. 9, 2021

(54) CHIRALLY ENRICHED OLIGOMERIC COMPOUNDS

(71) Applicant: Ionis Pharmaceuticals, Inc., Carlsbad, CA (US)

(72) Inventors: Punit P. Seth, Carlsbad, CA (US); Michael Oestergaard, Carlsbad, CA (US); Michael T. Migawa, Carlsbad, CA (US); Xue-hai Liang, Del Mar, CA (US); Wen Shen, Carlsbad, CA (US); Stanley T. Crooke, Carlsbad, CA (US); Eric E. Swayze, Encinitas, CA (US)

(73) Assignee: Ionis Pharmaceuticals, Inc., Carlsbad, CA (US)

(21) Appl. No.: 17/282,314

(22) PCT Filed: Oct. 4, 2019
(86) PCT No.: PCT/US2019/054667

§ 371 Date: Apr. 1, 2021

Related U.S. Application Data
(60) Provisional application No. 62/742,260, filed on Oct. 5, 2018.

(51) Int. Cl. C12N 15/113

(52) U.S. Cl. CPC C12N 15/113; C12N 2310/341; C12N 2310/11; C12N 2310/315; C12N 2310/322

(57) ABSTRACT

The present disclosure provides oligomeric compounds comprising a modified oligonucleotide having one or more chirally enriched phosphorothioate intemucleoside linkages. In certain embodiments, the modified oligonucleotide decreases expression of target mRNA.

### SEQUENCE LISTING

The present application is being filed along with a Sequence Listing in electronic format. The Sequence Listing is provided as a file entitled CORE0151WOSEQ_ST25.txt created Oct. 3, 2019 which is 20 kb in size. The information in the electronic format of the sequence listing is incorporated herein by reference in its entirety.

### FIELD

The present disclosure provides oligomeric compounds comprising a modified oligonucleotide having one or more chirally enriched phosphorothioate internucleoside linkages. In certain embodiments, the modified oligonucleotide decreases expression of target mRNA.

### BACKGROUND

The principle behind antisense technology is that an antisense compound hybridizes to a target nucleic acid and modulates the amount, activity, and/or function of the target nucleic acid. In one example, target RNA function is modulated via degradation by RNase H upon hybridization with a DNA-like antisense compound. Another example of modulation of gene expression by target degradation is RNA interference (RNAi). RNAi refers to antisense-mediated gene silencing through a mechanism that utilizes the RNA-induced silencing complex (RISC). Regardless of the specific mechanism, sequence specificity makes antisense compounds attractive as tools for target validation and gene functionalization, as well as therapeutics to selectively modulate the expression of genes involved in the pathogenesis of disease.

Antisense technology is an effective means for modulating the expression of one or more specific gene products and can therefore prove to be uniquely useful in a number of therapeutic, diagnostic, and research applications. Chemically modified nucleosides may be incorporated into antisense compounds to enhance one or more properties, such as nuclease resistance, pharmacokinetics, or affinity for a target nucleic acid.

### SUMMARY

The present disclosure provides chirally enriched populations of oligomeric compounds comprising modified oligonucleotides having a region of 2′-β-D-deoxyribosyl sugar moieties linked through phosphorothioate internucleoside linking groups chirally enriched in the (Sp) configuration or the (Rp) configuration. Controlling the chirality of one or more phosphorothioate internucleoside linkages can alter the properties of a modified oligonucleotide. For example, site specific introduction of (Sp) or (Rp) phosphorothioate internucleoside linkages into the central region of a modified oligonucleotide can enhance the properties of the modified oligonucleotide. For example, in certain embodiments, site specific introduction of (Sp) or (Rp) phosphorothioate internucleoside linkages into the central region of a modified oligonucleotide can mitigate toxicity of an otherwise cytotoxic modified oligonucleotide having a stereorandom configuration at the phosphorothioate internucleoside linkages.

Site specific introduction of (Sp) or (Rp) phosphorothioate internucleoside linkages into the central region of a modified oligonucleotide can alter the protein binding properties of the modified oligonucleotide. In certain embodiments, site specific introduction of (Sp) or (Rp) phosphorothioate internucleoside linkages into the central region of a modified oligonucleotide can alter the cleavage pattern of RNase H1 when the modified oligonucleotide binds to a target nucleic acid. In certain embodiments, site specific introduction of

(Sp) or (Rp) phosphorothioate internucleoside linkages into the central region of a modified oligonucleotide can increase the therapeutic index relative to an otherwise identifical modified oligonucleotide having a stereorandom configuration at the phosphorothioate internucleoside linkages.

### DETAILED DESCRIPTION

It is to be understood that both the foregoing general description and the following detailed description are exemplary and explanatory only and are not restrictive of the embodiments, as claimed. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting.

The section headings used herein are for organizational purposes only and are not to be construed as limiting the subject matter described. All documents, or portions of documents, cited in this application, including, but not limited to, patents, patent applications, articles, books, treatises, and GenBank and NCBI reference sequence records are hereby expressly incorporated by reference for the portions of the document discussed herein, as well as in their entirety.

It is understood that the sequence set forth in each SEQ ID NO contained herein is independent of any modification to a sugar moiety, an internucleoside linkage, or a nucleobase. As such, compounds defined by a SEQ ID NO may comprise, independently, one or more modifications to a sugar moiety, an internucleoside linkage, or a nucleobase.

As used herein, “2′-deoxyfuranosyl sugar moiety” or “2′-deoxyfuranosyl sugar” means a furanosyl sugar moiety having two hydrogens at the 2′-position. 2′-deoxyfuranosyl sugar moieties may be unmodified or modified and may be substituted at positions other than the 2′-position or unsubstituted. A β-D-2′-deoxyribosyl sugar moiety or 2′-β-D-deoxyribosyl sugar moiety in the context of an oligonucleotide is an unsubstituted, unmodified 2′-deoxyfuranosyl and is found in naturally occurring deoxyribonucleic acids (DNA).

As used herein, “2′-modified” in reference to a furanosyl sugar moiety or nucleoside comprising a furanosyl sugar moiety means the furanosyl sugar moiety comprises a substituent other than H or OH at the 2′-position of the furanosyl sugar moiety. 2′-modified furanosyl sugar moieties include non-bicyclic and bicyclic sugar moieties and may comprise, but are not required to comprise, additional substituents at other positions of the furanosyl sugar moiety.

As used herein, “2′-substituted” in reference to a furanosyl sugar moiety or nucleoside comprising a furanosyl sugar moiety means the furanosyl sugar moiety or nucleoside comprising the furanosyl sugar moiety comprises a substituent other than H or OH at the 2′-position and is a non-bicyclic furanosyl sugar moiety. 2′-substituted furanosyl sugar moieties do not comprise additional substituents at other positions of the furanosyl sugar moiety other than a nucleobase and/or internucleoside linkage(s) when in the context of an oligonucleotide.

As used herein, “administration” or “administering” refers to routes of introducing a compound or composition provided herein to a subject to perform its intended function. Examples of routes of administration that can be used include, but are not limited to, administration by inhalation, subcutaneous injection, intrathecal injection, and oral administration.

As used herein, “administered concomitantly” or “co-administration” means administration of two or more compounds in any manner in which the pharmacological effects of both are manifest in the patient. Concomitant administration does not require that both compounds be administered in a single pharmaceutical composition, in the same dosage form, by the same route of administration, or at the same time. The effects of both compounds need not manifest themselves at the same time. The effects need only be overlapping for a period of time and need not be coextensive. Concomitant administration or co-administration encompasses administration in parallel, sequentially, separate, or simultaneous administration.

As used herein, “animal” refers to a human or non-human animal, including, but not limited to, mice, rats, rabbits, dogs, cats, pigs, and non-human primates, including, but not limited to, monkeys and chimpanzees.

As used herein, “antisense activity” means any detectable and/or measurable change attributable to the hybridization of an antisense compound to its target nucleic acid. In certain embodiments, antisense activity is a decrease in the amount or expression of a target nucleic acid or protein encoded by such target nucleic acid compared to target nucleic acid levels or target protein levels in the absence of the antisense compound.

As used herein, “antisense compound” means a compound comprising an antisense oligonucleotide and optionally one or more additional features, such as a conjugate group or terminal group.

As used herein, “antisense oligonucleotide” means an oligonucleotide having a nucleobase sequence that is at least partially complementary to a target nucleic acid.

As used herein, “ameliorate” in reference to a treatment means improvement in at least one symptom relative to the same symptom in the absence of the treatment. In certain embodiments, amelioration is the reduction in the severity or frequency of a symptom or the delayed onset or slowing of progression in the severity or frequency of a symptom.

As used herein, “bicyclic nucleoside” or “BNA” means a nucleoside comprising a bicyclic sugar moiety. As used herein, “bicyclic sugar” or “bicyclic sugar moiety” means a modified sugar moiety comprising two rings, wherein the second ring is formed via a bridge connecting two of the atoms in the first ring thereby forming a bicyclic structure. In certain embodiments, the first ring of the bicyclic sugar moiety is a furanosyl moiety, and the bicyclic sugar moiety is a modified furanosyl sugar moiety. In certain embodiments, the bicyclic sugar moiety does not comprise a furanosyl moiety. As used herein, “cEt” or “constrained ethyl” means a bicyclic sugar moiety, wherein the first ring of the bicyclic sugar moiety is a ribosyl sugar moiety, the second ring of the bicyclic sugar is formed via a bridge connecting the 4′-carbon and the 2′-carbon, the bridge has the formula 4′-CH(CH3)-O-2′, and the methyl group of the bridge is in the S configuration. A cEt bicyclic sugar moiety is in the (β-D configuration.

As used herein, “coding region” in the context of an RNA means the portion of the RNA that is translated into an amino acid sequence. The coding region of an mRNA excludes the 5′-untranslated region and the 3′-untranslated region.

As used herein, “chirally enriched population” means a plurality of molecules of identical molecular formula, wherein the number or percentage of molecules within the population that contain a particular stereochemical configuration at a particular chiral center is greater than the number or percentage of molecules expected to contain the same particular stereochemical configuration at the same particular chiral center within the population if the particular chiral center were stereorandom. Chirally enriched populations of molecules having multiple chiral centers within each molecule may contain one or more sterorandom chiral centers. In certain embodiments, the molecules are modified oligonucleotides. In certain embodiments, the molecules are compounds comprising modified oligonucleotides.

As used herein, “complementary” in reference to an oligonucleotide or a region of an oligonucleotide means that at least 70% of the nucleobases of the entire oligonucleotide or the region of the oligonucleotide, respectively, and the nucleobases of another nucleic acid or one or more regions thereof are capable of hydrogen bonding with one another when the nucleobase sequence of the oligonucleotide and the other nucleic acid are aligned in opposing directions. Complementary nucleobases are nucleobase pairs that are capable of forming hydrogen bonds with one another. Complementary nucleobase pairs include adenine (A) and thymine (T), adenine (A) and uracil (U), cytosine (C) and guanine (G), 5-methyl cytosine (^{m}C) and guanine (G). Complementary oligonucleotides and/or nucleic acids need not have nucleobase complementarity at each nucleoside. Rather, some mismatches are tolerated. As used herein, “fully complementary” or “100% complementary” in reference to oligonucleotides means that such oligonucleotides are complementary to another oligonucleotide or nucleic acid at each nucleoside of the oligonucleotide.

As used herein, “conjugate group” means a group of atoms that is directly or indirectly attached to an oligonucleotide. Conjugate groups may comprise a conjugate moiety and a conjugate linker that attaches the conjugate moiety to the oligonucleotide.

As used herein, “conjugate linker” means a group of atoms comprising at least one bond that connects a conjugate moiety to an oligonucleotide.

As used herein, “conjugate moiety” means a group of atoms that is attached to an oligonucleotide via a conjugate linker.

As used herein, “contiguous” or “adjacent” in the context of an oligonucleotide refers to nucleosides, nucleobases, sugar moieties, or internucleoside linkages that are immediately adjacent to each other independent of the other moieties of the oligonucleotide. For example, “contiguous nucleobases” means nucleobases that are immediately adjacent to each other in a sequence. Moieties that are “directly linked” are immediately adjacent to each other and not separated by any other type of moiety.

As used herein, “degradation” in the context of a nucleic acid or protein means at least one cleavage of a contiguous nucleic acid or polypeptide. In certain embodiments, the at least one cleavage is performed by a nuclease.

As used herein, “double-stranded antisense compound” means an antisense compound comprising two oligomeric compounds that are complementary to each other and form a duplex, and wherein one of the two said oligomeric compounds comprises an antisense oligonucleotide.

As used herein, “effective amount” means the amount of compound sufficient to effectuate a desired physiological outcome in a subject in need of the compound. The effective amount may vary among subjects depending on the health and physical condition of the subject to be treated, the taxonomic group of the subjects to be treated, the formulation of the composition, assessment of the subject's medical condition, and other relevant factors.

As used herein, “efficacy” means the ability to produce a desired effect.

As used herein, “exon-exon junction” means a contiguous portion of an mRNA where two exons of a corresponding pre-mRNA were spliced together. An exon-exon junction includes at least one nucleoside of each of the two respective exons and may include up to the entirety of both of the respective exons.

As used herein, “expression” includes all the functions by which a gene's coded information is converted into structures present and operating in a cell. Such structures include, but are not limited to, the products of transcription and translation. As used herein, “modulation of expression” means any change in amount or activity of a product of transcription or translation of a gene. Such a change may be an increase or a reduction of any amount relative to the expression level prior to the modulation.

As used herein, “gapmer” means an oligonucleotide or a portion of an oligonucleotide having a central region comprising a plurality of nucleosides that support RNase H cleavage positioned between a 5′-region and a 3′-region. Herein, the 3′- and 5′-most nucleosides of the central region each comprise a 2′-deoxyfuranosyl sugar moiety. Herein, the 3′-most nucleoside of the 5′-region comprises a 2′-modified sugar moiety or a sugar surrogate. Herein, the 5′-most nucleoside of the 3′-region comprises a 2′-modified sugar moiety or a sugar surrogate. The “central region” may be referred to as a “gap”; and the “5′-region” and “3′-region” may be referred to as “wings”.

As used herein, “hybridization” means the pairing or annealing of complementary oligonucleotides and/or nucleic acids. While not limited to a particular mechanism, the most common mechanism of hybridization involves hydrogen bonding, which may be Watson-Crick, Hoogsteen or reversed Hoogsteen hydrogen bonding, between complementary nucleobases.

As used herein, “inhibiting the expression or activity” refers to a reduction or blockade of the expression or activity relative to the expression or activity in an untreated or control sample and does not necessarily indicate a total elimination of expression or activity. Inhibition of the expression or activity of a nucleic acid, such as a target mRNA, includes but is not limited to degradation of the nucleic acid.

As used herein, the terms “internucleoside linkage” means a group or bond that forms a covalent linkage between adjacent nucleosides in an oligonucleotide. As used herein “modified internucleoside linkage” means any internucleoside linkage other than a naturally occurring, phosphodiester internucleoside linkage. “Phosphorothioate linkage” means a modified internucleoside linkage in which one of the non-bridging oxygen atoms of a phosphodiester is replaced with a sulfur atom. Modified internucleoside linkages may or may not contain a phosphorus atom. A “neutral internucleoside linkage” is a modified internucleoside linkage that is mostly or completely uncharged at pH 7.4 and/or has a pKa below 7.4.

As used herein, “abasic nucleoside” means a sugar moiety in an oligonucleotide or oligomeric compound that is not directly connected to a nucleobase. In certain embodiments, an abasic nucleoside is adjacent to one or two nucleosides in an oligonucleotide.

As used herein, “LICA-1” is a conjugate group that is represented by the formula:

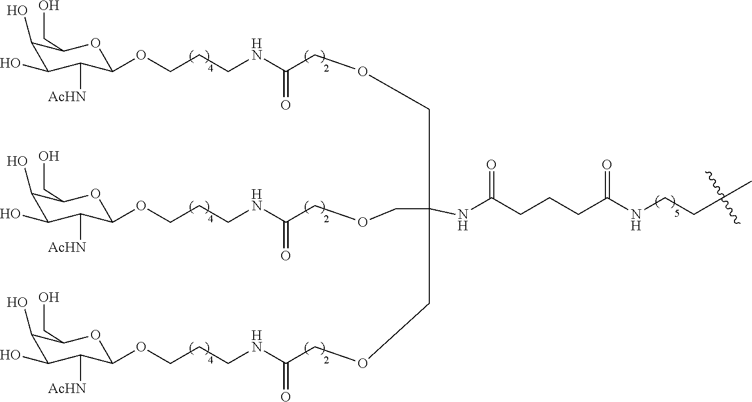

As used herein, “linker-nucleoside” means a nucleoside that links, either directly or indirectly, an oligonucleotide to a conjugate moiety. Linker-nucleosides are located within the conjugate linker of an oligomeric compound. Linker-nucleosides are not considered part of the oligonucleotide portion of an oligomeric compound even if they are contiguous with the oligonucleotide.

As used herein, “non-bicyclic sugar” or “non-bicyclic sugar moiety” means a sugar moiety that comprises fewer than 2 rings. Substituents of modified, non-bicyclic sugar moieties do not form a bridge between two atoms of the sugar moiety to form a second ring.

As used herein, “linked nucleosides” are nucleosides that are connected in a continuous sequence (i.e. no additional nucleosides are present between those that are linked).

As used herein, “mismatch” or “non-complementary” means a nucleobase of a first oligonucleotide that is not complementary with the corresponding nucleobase of a second oligonucleotide or target nucleic acid when the first and second oligomeric compound are aligned.

As used herein, “modulating” refers to changing or adjusting a feature in a cell, tissue, organ or organism.

As used herein, “MOE” means methoxyethyl. “2′-MOE” or “2′-O-methoxyethyl” means a 2′-OCH₂CH₂OCH₃ group at the 2′-position of a furanosyl ring. In certain embodiments, the 2′-OCH₂CH₂OCH₃ group is in place of the 2′-OH group of a ribosyl ring or in place of a 2′-H in a 2′-deoxyribosyl ring.

As used herein, “motif” means the pattern of unmodified and/or modified sugar moieties, nucleobases, and/or internucleoside linkages, in an oligonucleotide or a portion of an oligonucleotide.

As used herein, “naturally occurring” means found in nature.

As used herein, “nucleobase” means an unmodified nucleobase or a modified nucleobase. As used herein an “unmodified nucleobase” is adenine (A), thymine (T), cytosine (C), uracil (U), or guanine (G). As used herein, a modified nucleobase is a group of atoms capable of pairing with at least one unmodified nucleobase. A universal base is a nucleobase that can pair with any one of the five unmodified nucleobases. 5-methylcytosine (^{m}C) is one example of a modified nucleobase.

As used herein, “nucleobase sequence” means the order of contiguous nucleobases in a nucleic acid or oligonucleotide independent of any sugar moiety or internucleoside linkage modification.

As used herein, “nucleoside” means a moiety comprising a nucleobase and a sugar moiety. The nucleobase and sugar moiety are each, independently, unmodified or modified. As used herein, “modified nucleoside” means a nucleoside comprising a modified nucleobase and/or a modified sugar moiety.

As used herein, “oligomeric compound” means a compound consisting of an oligonucleotide and optionally one or more additional features, such as a conjugate group or terminal group.

As used herein, “oligonucleotide” means a strand of linked nucleosides connected via internucleoside linkages, wherein each nucleoside and internucleoside linkage may be modified or unmodified. Unless otherwise indicated, oligonucleotides consist of 8-50 linked nucleosides. As used herein, “modified oligonucleotide” means an oligonucleotide, wherein at least one nucleoside or internucleoside linkage is modified. As used herein, “unmodified oligonucleotide” means an oligonucleotide that does not comprise any nucleoside modifications or internucleoside modifications.

As used herein, “pharmaceutically acceptable carrier or diluent” means any substance suitable for use in administering to an animal. Certain such carriers enable pharmaceutical compositions to be formulated as, for example, liquids, powders, or suspensions that can be aerosolized or otherwise dispersed for inhalation by a subject. In certain embodiments, a pharmaceutically acceptable carrier or diluent is sterile water; sterile saline; or sterile buffer solution.

As used herein “pharmaceutically acceptable salts” means physiologically and pharmaceutically acceptable salts of compounds, such as oligomeric compounds, i.e., salts that retain the desired biological activity of the compound and do not impart undesired toxicological effects thereto.

As used herein “pharmaceutical composition” means a mixture of substances suitable for administering to a subject. For example, a pharmaceutical composition may comprise an antisense compound and an aqueous solution.

As used herein, “RNAi compound” means an antisense compound that acts, at least in part, through RISC or Ago2 to modulate a target nucleic acid and/or protein encoded by a target nucleic acid. RNAi compounds include, but are not limited to double-stranded siRNA, single-stranded RNA (ssRNA), and microRNA, including microRNA mimics. In certain embodiments, an RNAi compound modulates the amount, activity, and/or splicing of a target nucleic acid. The term RNAi compound excludes antisense oligonucleotides that act through RNase H.

As used herein, the term “single-stranded” in reference to an antisense compound means such a compound consisting of one oligomeric compound that is not paired with a second oligomeric compound to form a duplex. “Self-complementary” in reference to an oligonucleotide means an oligonucleotide that at least partially hybridizes to itself. A compound consisting of one oligomeric compound, wherein the oligonucleotide of the oligomeric compound is self-complementary, is a single-stranded compound. A single-stranded antisense or oligomeric compound may be capable of binding to a complementary oligomeric compound to form a duplex, in which case the compound would no longer be single-stranded.

As used herein, “standard cell assay” means an assay described in any of the Examples, and reasonable variations thereof.

As used herein, “stereorandom chiral center” in the context of a population of molecules of identical molecular formula means a chiral center having a random stereochemical configuration. For example, in a population of molecules comprising a stereorandom chiral center, the number of molecules having the (S) configuration of the stereorandom chiral center may be but is not necessarily the same as the number of molecules having the (R) configuration of the stereorandom chiral center. The stereochemical configuration of a chiral center is considered random when it is the result of a synthetic method that is not designed to control the stereochemical configuration. In certain embodiments, a stereorandom chiral center is a stereorandom phosphorothioate internucleoside linkage.

As used herein, “subject” means a human or non-human animal selected for treatment or therapy.

As used herein, “sugar moiety” means an unmodified sugar moiety or a modified sugar moiety. As used herein, “unmodified sugar moiety” means a β-D-ribosyl moiety, as found in naturally occurring RNA, or a β-D-2′-deoxyribosyl sugar moiety as found in naturally occurring DNA. As used herein, “modified sugar moiety” or “modified sugar” means a sugar surrogate or a furanosyl sugar moiety other than a β-D-ribosyl or a β-D-2′-deoxyribosyl. Modified furanosyl sugar moieties may be modified or substituted at a certain position(s) of the sugar moiety, or unsubstituted, and they may or may not have a stereoconfiguration other than β-D-ribosyl. Modified furanosyl sugar moieties include bicyclic sugars and non-bicyclic sugars. As used herein, “sugar surrogate” means a modified sugar moiety that does not comprise a furanosyl or tetrahydrofuranyl ring (is not a “furanosyl sugar moiety”) and that can link a nucleobase to another group, such as an internucleoside linkage, conjugate group, or terminal group in an oligonucleotide. Modified nucleosides comprising sugar surrogates can be incorporated into one or more positions within an oligonucleotide and such oligonucleotides are capable of hybridizing to complementary oligomeric compounds or nucleic acids.

As used herein, “target” in the context of a nucleic acid, such as an RNA, means a nucleic acid that an oligomeric compound is designed to affect. In certain embodiments, an oligomeric compound comprises an oligonucleotide having a nucleobase sequence that is complementary to more than one RNA, only one of which is the target RNA of the oligomeric compound. In certain embodiments, the target RNA is an RNA present in the species to which an oligomeric compound is administered. In certain embodiments, the target RNA is an mRNA. In certain such embodiments, the target mRNA is a mature mRNA, meaning that the mRNA has already been processed. A mature mRNA excludes a pre-mRNA.

As used herein, “therapeutically effective amount” means an amount of a compound, pharmaceutical agent, or composition that provides a therapeutic benefit to a subject.

As used herein, “treat” refers to administering a compound or pharmaceutical composition to an animal in order to effect an alteration or improvement of a disease, disorder, or condition in the animal.

As used herein, a “standard RNase H cleavage assay” is an assay wherein a heteroduplex of the modified oligonucleotide and a complementary strand of unmodified RNA are incubated with each other to form a heteroduplex, and are then incubated with RNase H1 for specified time points before being analyzed on a polyacrylamide gel.

As used herein, a modified nucleoside “supports RNase H cleavage” when incorporated into an oligonucleotide if RNase H cleavage of the complementary RNA is observed within two nucleobases of the modified nucleoside in a standard RNase H cleavage assay.

### Certain Compounds

In certain embodiments, compounds described herein are oligomeric compounds comprising or consisting of oligonucleotides consisting of linked nucleosides. Oligonucleotides may be unmodified oligonucleotides or may be modified oligonucleotides. Modified oligonucleotides comprise at least one modification relative to an unmodified oligonucleotide (i.e., comprise at least one modified nucleoside (comprising a modified sugar moiety and/or a modified nucleobase) and/or at least one modified internucleoside linkage).

### Certain Embodiments

The present disclosure provides the following non-limiting numbered embodiments:
Embodiment 1. A chirally enriched population of oligomeric compounds comprising modified oligonucleotides, wherein the modified oligonucleotides consist of 12-23 linked nucleosides, wherein the modified oligonucleotide comprises a gapmer consisting of a 5′-region, a central region, and a 3′-region wherein: the 5′-region consists of 1-5 linked modified nucleosides, wherein each nucleoside of the 5′-region comprises a 2′-modified furanosyl sugar moiety; the 3′-region consists of 1-5 linked modified nucleosides, wherein each nucleoside of the 3′-region comprises a 2′-modified furanosyl sugar moiety; the central region consists of 7-10 linked nucleosides, where each nucleoside of the central region comprises a 2′-β-D-deoxyribosyl sugar moiety; and wherein the central region has at least one phosphorothioate internucleoside linkage that is chirally enriched in the (Sp) configuration or the (Rp) configuration.
Embodiment 2. A chirally enriched population of oligomeric compounds, wherein the oligomeric compounds comprise a modified oligonucleotide consisting of 12-23 linked nucleosides, wherein the modified oligonucleotide is a gapmer consisting of a 5′-region, a central region, and a 3′-region wherein: the 5′-region consists of 1-5 linked modified nucleosides, wherein each nucleoside of the 5′-region comprises a 2′-modified furanosyl sugar moiety; the 3′-region consists of 1-5 linked modified nucleosides, wherein each nucleoside of the 3′-region comprises a 2′-modified furanosyl sugar moiety; the central region consists of 7-10 linked nucleosides, where each nucleoside of the central region comprises a 2′-β-D-deoxyribosyl sugar moiety; wherein the central region has at least one phosphorothioate internucleoside linkage; and wherein a percentage of oligomeric compounds within the population that contain a stereochemical configuration at the phosphorothioate internucleoside linkage is greater than an expected percentage of oligomeric compounds expected to contain the same particular stereochemical configuration at the phosphorothioate internucleoside linkage within the population if the stereochemical configuration at the phosphorothioate internucleoside linkage was stereorandom.
Embodiment 3. The chirally enriched population of oligomeric compounds of embodiment 1 or 2, wherein each central region internucleoside linkage is selected from among a phosphodiester internucleoside linkage and a phosphorothioate internucleoside linkage.
Embodiment 4. The chirally enriched population of oligomeric compounds of embodiment 1 or 2, wherein the central region has 2 phosphorothioate internucleoside linkages.
Embodiment 5. The chirally enriched population of oligomeric compounds of embodiment 1 or 2, wherein the central region has 3 phosphorothioate internucleoside linkages.
Embodiment 6. The chirally enriched population of oligomeric compounds of embodiment 1 or 2, wherein the central region has 4 phosphorothioate internucleoside linkages.
Embodiment 7. The chirally enriched population of oligomeric compounds of embodiment 1 or 2, wherein the central region has 5 phosphorothioate internucleoside linkages.
Embodiment 8. The chirally enriched population of oligomeric compounds of embodiment 1 or 2, wherein the central region has 6 phosphorothioate internucleoside linkages.
Embodiment 9. The chirally enriched population of oligomeric compounds of embodiment 1 or 2, wherein the central region has 7 phosphorothioate internucleoside linkages.
Embodiment 10. The chirally enriched population of oligomeric compounds of embodiment 1 or 2, wherein the central region has 8 phosphorothioate internucleoside linkages.

Embodiment 11. The chirally enriched population of oligomeric compounds of embodiment 1 or 2, wherein the central region has 9 phosphorothioate internucleoside linkages.
Embodiment 12. The chirally enriched population of oligomeric compounds of embodiment 1 or 2, wherein the central region has 10 phosphorothioate internucleoside linkages.
Embodiment 13. The chirally enriched population of oligomeric compounds of embodiment 1 or 2, wherein the central region has 11 phosphorothioate internucleoside linkages.
Embodiment 14. The chirally enriched population of oligomeric compounds of embodiment 1 or 2, wherein each central region internucleoside linkage is a phosphorothioate internucleoside linkage.
Embodiment 15. The chirally enriched population of oligomeric compounds of embodiment 1 or 2, wherein each phosphorothioate internucleoside linkage has the (Sp) configuration.
Embodiment 16. The chirally enriched population of oligomeric compounds of embodiment 1 or 2, wherein each phosphorothioate internucleoside linkage has the (Rp) configuration.
Embodiment 17. The chirally enriched population of oligomeric compounds of embodiment 1 or 2, wherein 1 central region internucleoside linkage has the (Rp) configuration.
Embodiment 18. The chirally enriched population of oligomeric compounds of any of embodiments 1-14, wherein 2 central region internucleoside linkages have the (Rp) configuration.
Embodiment 19. The chirally enriched population of oligomeric compounds of embodiment 18, wherein the 2 central region internucleoside linkages having the (Rp) configuration are adjacent.
Embodiment 20. The chirally enriched population of oligomeric compounds of embodiment 1 or 2, wherein the central region consists of 7 linked nucleosides and has the formula:
(N_{d})_{L1}(N_{d})_{L2}(N_{d})_{L3}(N_{d})_{L4}(N_{d})_{L5}(N_{d})_{L6}(N_{d})_{L7}; wherein each N_{d} is a 2′-β-D- deoxyribosyl sugar moiety and each of L1, L2, L3, L4, L5, L6, and L7 is a phosphorothioate internucleoside linkage; and wherein at least one of L1, L2, L3, L4, L5, L6, and L7 has the (Sp) configuration or the (Rp) configuration.
Embodiment 21. The chirally enriched population of oligomeric compounds of embodiment 20, wherein L1 is a phosphorothioate internucleoside linkage having the (Rp) configuration and where each of L2, L3, L4, L5, L6, and L7 is a phosphorothioate internucleoside linkage having the (Sp) configuration.
Embodiment 22. The chirally enriched population of oligomeric compounds of embodiment 20, wherein L2 is a phosphorothioate internucleoside linkage having the (Rp) configuration and where each of L1, L3, L4, L5, L6, and L7 is a phosphorothioate internucleoside linkage having the (Sp) configuration.
Embodiment 23. The chirally enriched population of oligomeric compounds of embodiment 20, wherein L3 is a phosphorothioate internucleoside linkage having the (Rp) configuration and where each of L1, L2, L4, L5, L6, and L7 is a phosphorothioate internucleoside linkage having the (Sp) configuration.
Embodiment 24. The chirally enriched population of oligomeric compounds of embodiment 20, wherein L4 is a phosphorothioate internucleoside linkage having the (Rp) configuration and where each of L1, L2, L3, L5, L6, and L7 is a phosphorothioate internucleoside linkage having the (Sp) configuration.
Embodiment 25. The chirally enriched population of oligomeric compounds of embodiment 20, wherein L5 is a phosphorothioate internucleoside linkage having the (Rp) configuration and where each of L1, L2, L3, L4, L6, and L7 is a phosphorothioate internucleoside linkage having the (Sp) configuration.
Embodiment 26. The chirally enriched population of oligomeric compounds of embodiment 20, wherein L6 is a phosphorothioate internucleoside linkage having the (Rp) configuration and where each of L1, L2, L3, L4, L5, and L7 is a phosphorothioate internucleoside linkage having the (Sp) configuration.
Embodiment 27. The chirally enriched population of oligomeric compounds of embodiment 20, wherein L7 is a phosphorothioate internucleoside linkage having the (Rp) configuration and where each of L1, L2, L3, L4, L5, and L6 is a phosphorothioate internucleoside linkage having the (Sp) configuration.
Embodiment 28. The chirally enriched population of oligomeric compounds of embodiment 20, wherein each L1 and L2 is a phosphorothioate internucleoside linkage having the (Rp) configuration and where each of L3, L4, L5, L6, and L7 is a phosphorothioate internucleoside linkage having the (Sp) configuration.
Embodiment 29. The chirally enriched population of oligomeric compounds of embodiment 20, wherein each L2 and L3 is a phosphorothioate internucleoside linkage having the (Rp) configuration and where each of L1, L4, L5, L6, and L7 is a phosphorothioate internucleoside linkage having the (Sp) configuration.
Embodiment 30. The chirally enriched population of oligomeric compounds of embodiment 20, wherein each L3 and L4 is a phosphorothioate internucleoside linkage having the (Rp) configuration and where each of L1, L2, L5, L6, and L7 is a phosphorothioate internucleoside linkage having the (Sp) configuration.
Embodiment 31. The chirally enriched population of oligomeric compounds of embodiment 20, wherein each L4 and L5 is a phosphorothioate internucleoside linkage having the (Rp) configuration and where each of L1, L2, L3, L6, and L7 is a phosphorothioate internucleoside linkage having the (Sp) configuration.
Embodiment 32. The chirally enriched population of oligomeric compounds of embodiment 20, wherein each L5 and L6 is a phosphorothioate internucleoside linkage having the (Rp) configuration and where each of L1, L2, L3, L4, and L7 is a phosphorothioate internucleoside linkage having the (Sp) configuration.
Embodiment 33. The chirally enriched population of oligomeric compounds of embodiment 20, wherein each L6 and L7 is a phosphorothioate internucleoside linkage having the (Rp) configuration and where each of L1, L2, L3, L4, and L5 is a phosphorothioate internucleoside linkage having the (Sp) configuration.
Embodiment 34. The chirally enriched population of oligomeric compounds of embodiment 20, wherein each L1 is a phosphorothioate internucleoside linkage having the (Rp) configuration, each L2 is a phosphorothioate internucleoside linkage having the (Sp) configuration, and where each of L3, L4, L5, L6 and L7 is a stereorandom phosphorothioate internucleoside linkage.
Embodiment 35. The chirally enriched population of oligomeric compounds of embodiment 20, wherein each L2 is a phosphorothioate internucleoside linkage having the (Rp) configuration, each L3 is a phosphorothioate internucleoside linkage having the (Sp) configuration, and where each of L1, L4, L5, L6 and L7 is a stereorandom phosphorothioate internucleoside linkage.
Embodiment 36. The chirally enriched population of oligomeric compounds of embodiment 20, wherein each L3 is a phosphorothioate internucleoside linkage having the (Rp) configuration, each L4 is a phosphorothioate internucleoside linkage having the (Sp) configuration, and where each of L1, L2, L5, L6 and L7 is a stereorandom phosphorothioate internucleoside linkage.
Embodiment 37. The chirally enriched population of oligomeric compounds of embodiment 20, wherein each L4 is a phosphorothioate internucleoside linkage having the (Rp) configuration, each L5 is a phosphorothioate internucleoside linkage having the (Sp) configuration, and where each of L1, L2, L3, L6 and L7 is a stereorandom phosphorothioate internucleoside linkage.
Embodiment 38. The chirally enriched population of oligomeric compounds of embodiment 20, wherein each L5 is a phosphorothioate internucleoside linkage having the (Rp) configuration, each L6 is a phosphorothioate internucleoside linkage having the (Sp) configuration, and where each of L1, L2, L3, L4 and L7 is a stereorandom phosphorothioate internucleoside linkage.
Embodiment 39. The chirally enriched population of oligomeric compounds of embodiment 20, wherein each L6 is a phosphorothioate internucleoside linkage having the (Rp) configuration, each L7 is a phosphorothioate internucleoside linkage having the (Sp) configuration, and where each of L1, L2, L3, L4 and L5 is a stereorandom phosphorothioate internucleoside linkage.
Embodiment 40. The oligomeric compound of embodiment 1 or 2, wherein the central region consists of 8 linked nucleosides and has the formula: (N_{d})_{L1}(N_{d})_{L2}(N_{d})_{L3}(N_{d})_{L4}(N_{d})_{L5}(N_{d})_{L6}(N_{d})_{L7}(N_{d})_{L8}; wherein each N_{d} is a 2′-β-D-deoxyribosyl sugar moiety and each of L1, L2, L3, L4, L5, L6, L7, and L8 is a phosphorothioate internucleoside linkage; and wherein at least one of L1, L2, L3, L4, L5, L6, L7 and L8 has the (Sp) configuration or the (Rp) configuration.
Embodiment 41. The oligomeric compound of embodiment 1 or 2, wherein the central region consists of 9 linked nucleosides and has the formula: (N_{d})_{L1}(N_{d})_{L2}(N_{d})_{L3}(N_{d})_{L4}(N_{d})_{L5}(N_{d})_{L6}(N_{d})_{L7}(N_{d})_{L8}(N_{d})_{L9}; wherein each N_{d} is a 2′-β-D-deoxyribosyl sugar moiety and each of L1, L2, L3, L4, L5, L6, L7, L8, and L9 is a phosphorothioate internucleoside linkage; and wherein at least one of L1, L2, L3, L4, L5, L6, L7, L8 and L9 has the (Sp) configuration or the (Rp) configuration.
Embodiment 42. The chirally enriched population of oligomeric compounds of embodiment 41, wherein L1 is a phosphorothioate internucleoside linkage having the (Rp) configuration and where each of L2, L3, L4, L5, L6, L7 and L8 is a phosphorothioate internucleoside linkage having the (Sp) configuration.
Embodiment 43. The chirally enriched population of oligomeric compounds of embodiment 41, wherein L2 is a phosphorothioate internucleoside linkage having the (Rp) configuration and where each of L1, L3, L4, L5, L6, L7 and L8 is a phosphorothioate internucleoside linkage having the (Sp) configuration.
Embodiment 44. The chirally enriched population of oligomeric compounds of embodiment 41, wherein L3 is a phosphorothioate internucleoside linkage having the (Rp) configuration and where each of L1, L2, L4, L5, L6, L7 and L8 is a phosphorothioate internucleoside linkage having the (Sp) configuration.
Embodiment 45. The chirally enriched population of oligomeric compounds of embodiment 41, wherein L4 is a phosphorothioate internucleoside linkage having the (Rp) configuration and where each of L1, L2, L4, L5, L6, L7 and L8 is a phosphorothioate internucleoside linkage having the (Sp) configuration.
Embodiment 46. The chirally enriched population of oligomeric compounds of embodiment 41, wherein L5 is a phosphorothioate internucleoside linkage having the (Rp) configuration and where each of L1, L2, L3, L4, L6, L7 and L8 is a phosphorothioate internucleoside linkage having the (Sp) configuration.
Embodiment 47. The chirally enriched population of oligomeric compounds of embodiment 41, wherein L6 is a phosphorothioate internucleoside linkage having the (Rp) configuration and where each of L1, L2, L3, L4, L5, L7 and L8 is a phosphorothioate internucleoside linkage having the (Sp) configuration.
Embodiment 48. The chirally enriched population of oligomeric compounds of embodiment 41, wherein L7 is a phosphorothioate internucleoside linkage having the (Rp) configuration and where each of L1, L2, L3, L4, L5, L6 and L8 is a phosphorothioate internucleoside linkage having the (Sp) configuration.
Embodiment 49. The chirally enriched population of oligomeric compounds of embodiment 41, wherein L8 is a phosphorothioate internucleoside linkage having the (Rp) configuration and where each of L1, L2, L3, L4, L5, L6 and L7 is a phosphorothioate internucleoside linkage having the (Sp) configuration.
Embodiment 50. The chirally enriched population of oligomeric compounds of embodiment 41, wherein each L1 and L2 is a phosphorothioate internucleoside linkage having the (Rp) configuration and where each of L3, L4, L5, L6 and L8 is a phosphorothioate internucleoside linkage having the (Sp) configuration.
Embodiment 51. The chirally enriched population of oligomeric compounds of embodiment 41, wherein each L2 and L3 is a phosphorothioate internucleoside linkage having the (Rp) configuration and where each of L1, L4, L5, L6 and L8 is a phosphorothioate internucleoside linkage having the (Sp) configuration.
Embodiment 52. The chirally enriched population of oligomeric compounds of embodiment 41, wherein each L3 and L4 is a phosphorothioate internucleoside linkage having the (Rp) configuration and where each of L1, L2, L5, L6 and L8 is a phosphorothioate internucleoside linkage having the (Sp) configuration.
Embodiment 53. The chirally enriched population of oligomeric compounds of embodiment 41, wherein each L4 and L5 is a phosphorothioate internucleoside linkage having the (Rp) configuration and where each of L1, L2, L3, L6 and L8 is a phosphorothioate internucleoside linkage having the (Sp) configuration.
Embodiment 54. The chirally enriched population of oligomeric compounds of embodiment 41, wherein each L5 and L6 is a phosphorothioate internucleoside linkage having the (Rp) configuration and where each of L1, L2, L3, L6 and L8 is a phosphorothioate internucleoside linkage having the (Sp) configuration.
Embodiment 55. The chirally enriched population of oligomeric compounds of embodiment 41, wherein each L6 and L7 is a phosphorothioate internucleoside linkage having the (Rp) configuration and where each of L1, L2, L3, L5 and L8 is a phosphorothioate internucleoside linkage having the (Sp) configuration.
Embodiment 56. The chirally enriched population of oligomeric compounds of embodiment 41, wherein each L7 and L8 is a phosphorothioate internucleoside linkage having the (Rp) configuration and where each of L1, L2, L3, L5 and L6 is a phosphorothioate internucleoside linkage having the (Sp) configuration.
Embodiment 57. The chirally enriched population of oligomeric compounds of embodiment 41, wherein each L1 is a phosphorothioate internucleoside linkage having the (Rp) configuration, each L2 is a phosphorothioate internucleoside linkage having the (Sp) configuration, and where each of L3, L4, L5, L6, L7 and L8 is a stereorandom phosphorothioate internucleoside linkage.
Embodiment 58. The chirally enriched population of oligomeric compounds of embodiment 41, wherein each L2 is a phosphorothioate internucleoside linkage having the (Rp) configuration, each L3 is a phosphorothioate internucleoside linkage having the (Sp) configuration, and where each of L1, L4, L5, L6, L7 and L8 is a stereorandom phosphorothioate internucleoside linkage.
Embodiment 59. The chirally enriched population of oligomeric compounds of embodiment 41, wherein each L3 is a phosphorothioate internucleoside linkage having the (Rp) configuration, each L4 is a phosphorothioate internucleoside linkage having the (Sp) configuration, and where each of L1, L2, L5, L6, L7 and L8 is a stereorandom phosphorothioate internucleoside linkage.
Embodiment 60. The chirally enriched population of oligomeric compounds of embodiment 41, wherein each L4 is a phosphorothioate internucleoside linkage having the (Rp) configuration, each L5 is a phosphorothioate internucleoside linkage having the (Sp) configuration, and where each of L1, L2, L3, L6, L7 and L8 is a stereorandom phosphorothioate internucleoside linkage.
Embodiment 61. The chirally enriched population of oligomeric compounds of embodiment 41, wherein each L5 is a phosphorothioate internucleoside linkage having the (Rp) configuration, each L6 is a phosphorothioate internucleoside linkage having the (Sp) configuration, and where each of L1, L2, L3, L4, L7 and L8 is a stereorandom phosphorothioate internucleoside linkage.
Embodiment 62. The chirally enriched population of oligomeric compounds of embodiment 41, wherein each L6 is a phosphorothioate internucleoside linkage having the (Rp) configuration, each L7 is a phosphorothioate internucleoside linkage having the (Sp) configuration, and where each of L1, L2, L3, L4, L5 and L8 is a stereorandom phosphorothioate internucleoside linkage.
Embodiment 63. The chirally enriched population of oligomeric compounds of embodiment 41, wherein each L7 is a phosphorothioate internucleoside linkage having the (Rp) configuration, each L8 is a phosphorothioate internucleoside linkage having the (Sp) configuration, and where each of L1, L2, L3, L4, L5 and L6 is a stereorandom phosphorothioate internucleoside linkage.
Embodiment 64. The oligomeric compound of embodiment 1 or 2, wherein the central region consists of 9 linked nucleosides and has the formula: (N_{d})_{L1}(N_{d})_{L2}(N_{d})_{L3}(N_{d})_{L4}(N_{d})_{L5}(N_{d})_{L6}(N_{d})_{L7}(N_{d})_{L8}(N_{d})_{L9}; wherein each N_{d} is a 2′-β-D-deoxyribosyl sugar moiety and each of L1, L2, L3, L4, L5, L6, L7, L8, and L9 is a phosphorothioate internucleoside linkage; and wherein at least one of L1, L2, L3, L4, L5, L6, L7, L8 and L9 has the (Sp) configuration or the (Rp) configuration.
Embodiment 65. The chirally enriched population of oligomeric compounds of embodiment 51 or 64, wherein L1 is a phosphorothioate internucleoside linkage having the (Rp) configuration and where each of L2, L3, L4, L5, L6, L7, L8 and L9 is a phosphorothioate internucleoside linkage having the (Sp) configuration.
Embodiment 66. The chirally enriched population of oligomeric compounds of embodiment 64, wherein L2 is a phosphorothioate internucleoside linkage having the (Rp) configuration and where each of L1, L3, L4, L5, L6, L7, L8 and L9 is a phosphorothioate internucleoside linkage having the (Sp) configuration.
Embodiment 67. The chirally enriched population of oligomeric compounds of embodiment 64, wherein L3 is a phosphorothioate internucleoside linkage having the (Rp) configuration and where each of L1, L2, L4, L5, L6, L7, L8 and L9 is a phosphorothioate internucleoside linkage having the (Sp) configuration.
Embodiment 68. The chirally enriched population of oligomeric compounds of embodiment 64, wherein L4 is a phosphorothioate internucleoside linkage having the (Rp) configuration and where each of L1, L2, L3, L5, L6, L7, L8 and L9 is a phosphorothioate internucleoside linkage having the (Sp) configuration.
Embodiment 69. The chirally enriched population of oligomeric compounds of embodiment 64, wherein L5 is a phosphorothioate internucleoside linkage having the (Rp) configuration and where each of L1, L2, L3, L4, L6, L7, L8 and L9 is a phosphorothioate internucleoside linkage having the (Sp) configuration.
Embodiment 70. The chirally enriched population of oligomeric compounds of embodiment 64, wherein L6 is a phosphorothioate internucleoside linkage having the (Rp) configuration and where each of L1, L2, L3, L4, L5, L7, L8 and L9 is a phosphorothioate internucleoside linkage having the (Sp) configuration.
Embodiment 71. The chirally enriched population of oligomeric compounds of embodiment 64, wherein L7 is a phosphorothioate internucleoside linkage having the (Rp) configuration and where each of L1, L2, L3, L4, L5, L6, L8 and L9 is a phosphorothioate internucleoside linkage having the (Sp) configuration.
Embodiment 72. The chirally enriched population of oligomeric compounds of embodiment 64, wherein L8 is a phosphorothioate internucleoside linkage having the (Rp) configuration and where each of L1, L2, L3, L4, L5, L6, L7 and L9 is a phosphorothioate internucleoside linkage having the (Sp) configuration.
Embodiment 73. The chirally enriched population of oligomeric compounds of embodiment 64, wherein L9 is a phosphorothioate internucleoside linkage having the (Rp) configuration and where each of L1, L2, L3, L4, L5, L6, L7 and L8 is a phosphorothioate internucleoside linkage having the (Sp) configuration.
Embodiment 74. The chirally enriched population of oligomeric compounds of embodiment 64, wherein each of L1 and L2 is a phosphorothioate internucleoside linkage having the (Rp) configuration and where each of L3, L4, L5, L6, L7, L8 and L9 is a phosphorothioate internucleoside linkage having the (Sp) configuration.
Embodiment 75. The chirally enriched population of oligomeric compounds of embodiment 64, wherein each of L2 and L3 is a phosphorothioate internucleoside linkage having the (Rp) configuration and where each of L1, L4, L5, L6, L7, L8 and L9 is a phosphorothioate internucleoside linkage having the (Sp) configuration.
Embodiment 76. The chirally enriched population of oligomeric compounds of embodiment 64, wherein each of L3 and L4 is a phosphorothioate internucleoside linkage having the (Rp) configuration and where each of L1, L2, L5, L6, L7, L8 and L9 is a phosphorothioate internucleoside linkage having the (Sp) configuration.
Embodiment 77. The chirally enriched population of oligomeric compounds of embodiment 64, wherein each of L4 and L5 is a phosphorothioate internucleoside linkage having the (Rp) configuration and where each of L1, L2, L3, L6, L7, L8 and L9 is a phosphorothioate internucleoside linkage having the (Sp) configuration.
Embodiment 78. The chirally enriched population of oligomeric compounds of embodiment 64, wherein each of L5 and L6 is a phosphorothioate internucleoside linkage having the (Rp) configuration and where each of L1, L2, L3, L4, L7, L8 and L9 is a phosphorothioate internucleoside linkage having the (Sp) configuration.
Embodiment 79. The chirally enriched population of oligomeric compounds of embodiment 64, wherein each of L6 and L7 is a phosphorothioate internucleoside linkage having the (Rp) configuration and where each of L1, L2, L3, L4, L5, L8 and L9 is a phosphorothioate internucleoside linkage having the (Sp) configuration.
Embodiment 80. The chirally enriched population of oligomeric compounds of embodiment 64, wherein each of L7 and L8 is a phosphorothioate internucleoside linkage having the (Rp) configuration and where each of L1, L2, L3, L4, L5, L6 and L9 is a phosphorothioate internucleoside linkage having the (Sp) configuration.
Embodiment 81. The chirally enriched population of oligomeric compounds of embodiment 64, wherein each of L8 and L9 is a phosphorothioate internucleoside linkage having the (Rp) configuration and where each of L1, L2, L3, L4, L5, L6 and L7 is a phosphorothioate internucleoside linkage having the (Sp) configuration.
Embodiment 82. The chirally enriched population of oligomeric compounds of embodiment 64, wherein each L1 is a phosphorothioate internucleoside linkage having the (Rp) configuration, each L2 is a phosphorothioate internucleoside linkage having the (Sp) configuration, and where each of L3, L4, L5, L6, L7, L8 and L9 is a stereorandom phosphorothioate internucleoside linkage.
Embodiment 83. The chirally enriched population of oligomeric compounds of embodiment 64, wherein each L2 is a phosphorothioate internucleoside linkage having the (Rp) configuration, each L3 is a phosphorothioate internucleoside linkage having the (Sp) configuration, and where each of L1, L4, L5, L6, L7, L8 and L9 is a stereorandom phosphorothioate internucleoside linkage.
Embodiment 84. The chirally enriched population of oligomeric compounds of embodiment 64, wherein each

L3 is a phosphorothioate internucleoside linkage having the (Rp) configuration, each L4 is a phosphorothioate internucleoside linkage having the (Sp) configuration, and where each of L1, L2, L5, L6, L7, L8 and L9 is a stereorandom phosphorothioate internucleoside linkage.
Embodiment 85. The chirally enriched population of oligomeric compounds of embodiment 64, wherein each L4 is a phosphorothioate internucleoside linkage having the (Rp) configuration, each L5 is a phosphorothioate internucleoside linkage having the (Sp) configuration, and where each of L1, L2, L3, L6, L7, L8 and L9 is a stereorandom phosphorothioate internucleoside linkage.
Embodiment 86. The chirally enriched population of oligomeric compounds of embodiment 64, wherein each L5 is a phosphorothioate internucleoside linkage having the (Rp) configuration, each L6 is a phosphorothioate internucleoside linkage having the (Sp) configuration, and where each of L1, L2, L3, L4, L7, L8 and L9 is a stereorandom phosphorothioate internucleoside linkage.
Embodiment 87. The chirally enriched population of oligomeric compounds of embodiment 64, wherein each L6 is a phosphorothioate internucleoside linkage having the (Rp) configuration, each L7 is a phosphorothioate internucleoside linkage having the (Sp) configuration, and where each of L1, L2, L3, L4, L5, L8 and L9 is a stereorandom phosphorothioate internucleoside linkage.
Embodiment 88. The chirally enriched population of oligomeric compounds of embodiment 64, wherein each L7 is a phosphorothioate internucleoside linkage having the (Rp) configuration, each L8 is a phosphorothioate internucleoside linkage having the (Sp) configuration, and where each of L1, L2, L3, L4, L5, L6 and L9 is a stereorandom phosphorothioate internucleoside linkage.
Embodiment 89. The chirally enriched population of oligomeric compounds of embodiment 64, wherein each

L8 is a phosphorothioate internucleoside linkage having the (Rp) configuration, each L9 is a phosphorothioate internucleoside linkage having the (Sp) configuration, and where each of L1, L2, L3, L4, L5, L6 and L7 is a stereorandom phosphorothioate internucleoside linkage.
Embodiment 90. The oligomeric compound of embodiment 1 or 2, wherein the central region consists of 10 linked nucleosides and has the formula: (N_{d})_{L1}(N_{d})_{L2}(N_{d})_{L3}(N_{d})_{L4}(N_{d})_{L5}(N_{d})_{L6}(N_{d})_{L7}(N_{d})_{L8}(N_{d})_{L9}(N_{d})_{L10}; wherein each N_{d} is a 2′-β-D-deoxyribosyl sugar moiety and each of L1, L2, L3, L4, L5, L6, L7, L8, L9, and L10 is a phosphorothioate internucleoside linkage; and wherein at least one of L1, L2, L3, L4, L5, L6, L7, L8, L9 and L10 has the (Sp) configuration or the (Rp) configuration.
Embodiment 91. The chirally enriched population of oligomeric compounds of embodiment 69 or 90, wherein L1 is a phosphorothioate internucleoside linkage having the (Rp) configuration and where each of L2, L3, L4, L5, L6, L7, L8, L9 and L10 is a phosphorothioate internucleoside linkage having the (Sp) configuration.
Embodiment 92. The chirally enriched population of oligomeric compounds of embodiment 90, wherein L2 is a phosphorothioate internucleoside linkage having the (Rp) configuration and where each of L1, L3, L4, L5, L6, L7, L8, L9 and L10 is a phosphorothioate internucleoside linkage having the (Sp) configuration.
Embodiment 93. The chirally enriched population of oligomeric compounds of embodiment 90, wherein L3 is a phosphorothioate internucleoside linkage having the (Rp) configuration and where each of L1, L2, L4, L5, L6, L7, L8, L9 and L10 is a phosphorothioate internucleoside linkage having the (Sp) configuration.
Embodiment 94. The chirally enriched population of oligomeric compounds of embodiment 90, wherein L4 is a phosphorothioate internucleoside linkage having the (Rp) configuration and where each of L1, L2, L3, L5, L6, L7, L8, L9 and L10 is a phosphorothioate internucleoside linkage having the (Sp) configuration.
Embodiment 95. The chirally enriched population of oligomeric compounds of embodiment 90, wherein L5 is a phosphorothioate internucleoside linkage having the (Rp) configuration and where each of L1, L2, L3, L4, L6, L7, L8, L9 and L10 is a phosphorothioate internucleoside linkage having the (Sp) configuration.
Embodiment 96. The chirally enriched population of oligomeric compounds of embodiment 90, wherein L6 is a phosphorothioate internucleoside linkage having the (Rp) configuration and where each of L1, L2, L3, L4, L5, L7, L8, L9 and L10 is a phosphorothioate internucleoside linkage having the (Sp) configuration.
Embodiment 97. The chirally enriched population of oligomeric compounds of embodiment 90, wherein L7 is a phosphorothioate internucleoside linkage having the (Rp) configuration and where each of L1, L2, L3, L4, L5, L6, L8, L9 and L10 is a phosphorothioate internucleoside linkage having the (Sp) configuration.
Embodiment 98. The chirally enriched population of oligomeric compounds of embodiment 90, wherein L8 is a phosphorothioate internucleoside linkage having the (Rp) configuration and where each of L1, L2, L3, L4, L5, L6, L7, L9 and L10 is a phosphorothioate internucleoside linkage having the (Sp) configuration.
Embodiment 99. The chirally enriched population of oligomeric compounds of embodiment 90, wherein L9 is a phosphorothioate internucleoside linkage having the (Rp) configuration and where each of L1, L2, L3, L4, L5, L6, L7, L8 and L10 is a phosphorothioate internucleoside linkage having the (Sp) configuration.
Embodiment 100. The chirally enriched population of oligomeric compounds of embodiment 90, wherein L10 is a phosphorothioate internucleoside linkage having the (Rp) configuration and where each of L1, L2, L3, L4, L5, L6, L7, L8 and L9 is a phosphorothioate internucleoside linkage having the (Sp) configuration.
Embodiment 101. The chirally enriched population of oligomeric compounds of embodiment 90, wherein each of L1 and L2 is a phosphorothioate internucleoside linkage having the (Rp) configuration and where each of L3, L4, L5, L6, L7, L8, L9 and L10 is a phosphorothioate internucleoside linkage having the (Sp) configuration.
Embodiment 102. The chirally enriched population of oligomeric compounds of embodiment 90, wherein each of L2 and L3 is a phosphorothioate internucleoside linkage having the (Rp) configuration and where each of L1, L4, L5, L6, L7, L8, L9 and L10 is a phosphorothioate internucleoside linkage having the (Sp) configuration.
Embodiment 103. The chirally enriched population of oligomeric compounds of embodiment 90, wherein each of L3 and L4 is a phosphorothioate internucleoside linkage having the (Rp) configuration and where each of L1, L2, L5, L6, L7, L8, L9 and L10 is a phosphorothioate internucleoside linkage having the (Sp) configuration.
Embodiment 104. The chirally enriched population of oligomeric compounds of embodiment 90, wherein each of L4 and L5 is a phosphorothioate internucleoside linkage having the (Rp) configuration and where each of L1, L2, L3, L6, L7, L8, L9 and L10 is a phosphorothioate internucleoside linkage having the (Sp) configuration.
Embodiment 105. The chirally enriched population of oligomeric compounds of embodiment 90, wherein each of L5 and L6 is a phosphorothioate internucleoside linkage having the (Rp) configuration and where each of L1, L2, L3, L4, L7, L8, L9 and L10 is a phosphorothioate internucleoside linkage having the (Sp) configuration.
Embodiment 106. The chirally enriched population of oligomeric compounds of embodiment 90, wherein each of L6 and L7 is a phosphorothioate internucleoside linkage having the (Rp) configuration and where each of L1, L2, L3, L4, L5, L8, L9 and L10 is a phosphorothioate internucleoside linkage having the (Sp) configuration.
Embodiment 107. The chirally enriched population of oligomeric compounds of embodiment 90, wherein each of L7 and L8 is a phosphorothioate internucleoside linkage having the (Rp) configuration and where each of L1, L2, L3, L4, L5, L6, L9 and L10 is a phosphorothioate internucleoside linkage having the (Sp) configuration.
Embodiment 108. The chirally enriched population of oligomeric compounds of embodiment 90, wherein each of L8 and L9 is a phosphorothioate internucleoside linkage having the (Rp) configuration and where each of L1, L2, L3, L4, L5, L6, L7 and L10 is a phosphorothioate internucleoside linkage having the (Sp) configuration.
Embodiment 109. The chirally enriched population of oligomeric compounds of embodiment 90, wherein each of L9 and L10 is a phosphorothioate internucleoside linkage having the (Rp) configuration and where each of L1, L2, L3, L4, L5, L6, L7 and L8 is a phosphorothioate internucleoside linkage having the (Sp) configuration.
Embodiment 110. The chirally enriched population of oligomeric compounds of embodiment 90, wherein each of L3, L6, L9 and L10 is a phosphorothioate internucleoside linkage having the (Rp) configuration and where each of L1, L2, L4, L5, L7, and L8 is a phosphorothioate internucleoside linkage having the (Sp) configuration.
Embodiment 111. The chirally enriched population of oligomeric compounds of embodiment 90, wherein each L1 is a phosphorothioate internucleoside linkage having the (Rp) configuration, each L2 is a phosphorothioate internucleoside linkage having the (Sp) configuration, and where each of L3, L4, L5, L6, L7, L8, L9 and L10 is a stereorandom phosphorothioate internucleoside linkage.
Embodiment 112. The chirally enriched population of oligomeric compounds of embodiment 90, wherein each L2 is a phosphorothioate internucleoside linkage having the (Rp) configuration, each L3 is a phosphorothioate internucleoside linkage having the (Sp) configuration, and where each of L1, L4, L5, L6, L7, L8, L9 and L10 is a stereorandom phosphorothioate internucleoside linkage.
Embodiment 113. The chirally enriched population of oligomeric compounds of embodiment 90, wherein each L3 is a phosphorothioate internucleoside linkage having the (Rp) configuration, each L4 is a phosphorothioate internucleoside linkage having the (Sp) configuration, and where each of L1, L2, L5, L6, L7, L8, L9 and L10 is a stereorandom phosphorothioate internucleoside linkage.
Embodiment 114. The chirally enriched population of oligomeric compounds of embodiment 90, wherein each L4 is a phosphorothioate internucleoside linkage having the (Rp) configuration, each L5 is a phosphorothioate internucleoside linkage having the (Sp) configuration, and where each of L1, L2, L3, L6, L7, L8, L9 and L10 is a stereorandom phosphorothioate internucleoside linkage.
Embodiment 115. The chirally enriched population of oligomeric compounds of embodiment 90, wherein each L5 is a phosphorothioate internucleoside linkage having the (Rp) configuration, each L6 is a phosphorothioate internucleoside linkage having the (Sp) configuration, and where each of L1, L2, L3, L4, L7, L8, L9 and L10 is a stereorandom phosphorothioate internucleoside linkage.
Embodiment 116. The chirally enriched population of oligomeric compounds of embodiment 90, wherein each L6 is a phosphorothioate internucleoside linkage having the (Rp) configuration, each L7 is a phosphorothioate internucleoside linkage having the (Sp) configuration, and where each of L1, L2, L3, L4, L5, L8, L9 and L10 is a stereorandom phosphorothioate internucleoside linkage.
Embodiment 117. The chirally enriched population of oligomeric compounds of embodiment 90, wherein each L7 is a phosphorothioate internucleoside linkage having the (Rp) configuration, each L8 is a phosphorothioate internucleoside linkage having the (Sp) configuration, and where each of L1, L2, L3, L4, L5, L6, L9 and L10 is a stereorandom phosphorothioate internucleoside linkage.
Embodiment 118. The chirally enriched population of oligomeric compounds of embodiment 90, wherein each L8 is a phosphorothioate internucleoside linkage having the (Rp) configuration, each L9 is a phosphorothioate internucleoside linkage having the (Sp) configuration, and where each of L1, L2, L3, L4, L5, L6, L7 and L10 is a stereorandom phosphorothioate internucleoside linkage.
Embodiment 119. The chirally enriched population of oligomeric compounds of embodiment 90, wherein each L9 is a phosphorothioate internucleoside linkage having the (Rp) configuration, each L10 is a phosphorothioate internucleoside linkage having the (Sp) configuration, and where each of L1, L2, L3, L4, L5, L6, L7 and L8 is a stereorandom phosphorothioate internucleoside linkage. Embodiment 120. The oligomeric compound of embodiment 1 or 2, wherein the central region consists of 11 linked nucleosides and has the formula:
(Nd)L1(Nd)L2(Nd)L3(Nd)L4(Nd)L5(Nd)L6(Nd)L7(Nd)L8(Nd)L9(Nd)L10(Nd)L11; wherein, each Nd is a 2′-β-D-deoxyribosyl sugar moiety and each of L1, L2, L3, L4, L5, L6, L7, L8, L9, L10 and L11 is a phosphorothioate internucleoside linkage; and wherein at least one of L1, L2, L3, L4, L5, L6, L7, L8, L9, L10 and L11 has the (Sp) configuration or the (Rp) configuration.
Embodiment 121. The chirally enriched population of oligomeric compounds of embodiment 120, wherein L1 is a phosphorothioate internucleoside linkage having the (Rp) configuration and where each of L2, L3, L4, L5, L6, L7, L8, L9, L10 and L11 is a phosphorothioate internucleoside linkage having the (Sp) configuration.
Embodiment 122. The chirally enriched population of oligomeric compounds of embodiment 120, wherein L2 is a phosphorothioate internucleoside linkage having the (Rp) configuration and where each of L1, L3, L4, L5, L6, L7, L8, L9, L10 and L11 is a phosphorothioate internucleoside linkage having the (Sp) configuration.
Embodiment 123. The chirally enriched population of oligomeric compounds of embodiment 120, wherein L3 is a phosphorothioate internucleoside linkage having the (Rp) configuration and where each of L1, L2, L4, L5, L6, L7, L8, L9, L10 and L11 is a phosphorothioate internucleoside linkage having the (Sp) configuration.
Embodiment 124. The chirally enriched population of oligomeric compounds of embodiment 120, wherein L4 is a phosphorothioate internucleoside linkage having the (Rp) configuration and where each of L1, L2, L3, L5, L6, L7, L8, L9, L10 and L11 is a phosphorothioate internucleoside linkage having the (Sp) configuration.
Embodiment 125. The chirally enriched population of oligomeric compounds of embodiment 120, wherein L5 is a phosphorothioate internucleoside linkage having the (Rp) configuration and where each of L1, L2, L3, L4, L6, L7, L8, L9, L10 and L11 is a phosphorothioate internucleoside linkage having the (Sp) configuration.
Embodiment 126. The chirally enriched population of oligomeric compounds of embodiment 120, wherein L6 is a phosphorothioate internucleoside linkage having the (Rp) configuration and where each of L1, L2, L3, L4, L5, L7, L8, L9, L10 and L11 is a phosphorothioate internucleoside linkage having the (Sp) configuration.
Embodiment 127. The chirally enriched population of oligomeric compounds of embodiment 120, wherein L7 is a phosphorothioate internucleoside linkage having the (Rp) configuration and where each of L1, L2, L3, L4, L5, L6, L8, L9, L10 and L11 is a phosphorothioate internucleoside linkage having the (Sp) configuration.
Embodiment 128. The chirally enriched population of oligomeric compounds of embodiment 120, wherein L8 is a phosphorothioate internucleoside linkage having the (Rp) configuration and where each of L1, L2, L3, L4, L5, L6, L7, L9, L10 and L11 is a phosphorothioate internucleoside linkage having the (Sp) configuration.
Embodiment 129. The chirally enriched population of oligomeric compounds of embodiment 120, wherein L9 is a phosphorothioate internucleoside linkage having the (Rp) configuration and where each of L1, L2, L3, L4, L5, L6, L7, L8, L10 and L11 is a phosphorothioate internucleoside linkage having the (Sp) configuration.
Embodiment 130. The chirally enriched population of oligomeric compounds of embodiment 120, wherein L10 is a phosphorothioate internucleoside linkage having the (Rp) configuration and where each of L1, L2, L3, L4, L5, L6, L7, L8, L9 and L11 is a phosphorothioate internucleoside linkage having the (Sp) configuration.
Embodiment 131. The chirally enriched population of oligomeric compounds of embodiment 120, wherein L11 is a phosphorothioate internucleoside linkage having the (Rp) configuration and where each of L1, L2, L3, L4, L5, L6, L7, L8, L9 and L10 is a phosphorothioate internucleoside linkage having the (Sp) configuration.
Embodiment 132. The chirally enriched population of oligomeric compounds of embodiment 120, wherein each of L1 and L2 is a phosphorothioate internucleoside linkage having the (Rp) configuration and where each of L3, L4, L5, L6, L7, L8, L9, L10 and L11 is a phosphorothioate internucleoside linkage having the (Sp) configuration.
Embodiment 133. The chirally enriched population of oligomeric compounds of embodiment 120, wherein each of L2 and L3 is a phosphorothioate internucleoside linkage having the (Rp) configuration and where each of L1, L4, L5, L6, L7, L8, L9, L10 and L11 is a phosphorothioate internucleoside linkage having the (Sp) configuration.
Embodiment 134. The chirally enriched population of oligomeric compounds of embodiment 120, wherein each of L2 and L3 is a phosphorothioate internucleoside linkage having the (Rp) configuration and where each of L1, L4, L5, L6, L7, L8, L9, L10 and L11 is a phosphorothioate internucleoside linkage having the (Sp) configuration.
Embodiment 135. The chirally enriched population of oligomeric compounds of embodiment 120, wherein each of L3 and L4 is a phosphorothioate internucleoside linkage having the (Rp) configuration and where each of L1, L2, L5, L6, L7, L8, L9, L10 and L11 is a phosphorothioate internucleoside linkage having the (Sp) configuration.
Embodiment 136. The chirally enriched population of oligomeric compounds of embodiment 120, wherein each of L4 and L5 is a phosphorothioate internucleoside linkage having the (Rp) configuration and where each of L1, L2, L3, L6, L7, L8, L9, L10 and L11 is a phosphorothioate internucleoside linkage having the (Sp) configuration.
Embodiment 137. The chirally enriched population of oligomeric compounds of embodiment 120, wherein each of L5 and L6 is a phosphorothioate internucleoside linkage having the (Rp) configuration and where each of L1, L2, L3, L4, L7, L8, L9, L10 and L11 is a phosphorothioate internucleoside linkage having the (Sp) configuration.
Embodiment 138. The chirally enriched population of oligomeric compounds of embodiment 120, wherein each of L6 and L7 is a phosphorothioate internucleoside linkage having the (Rp) configuration and where each of L1, L2, L3, L4, L5, L8, L9, L10 and L11 is a phosphorothioate internucleoside linkage having the (Sp) configuration.
Embodiment 139. The chirally enriched population of oligomeric compounds of embodiment 120, wherein each of L7 and L8 is a phosphorothioate internucleoside linkage having the (Rp) configuration and where each of L1, L2, L3, L4, L5, L6, L9, L10 and L11 is a phosphorothioate internucleoside linkage having the (Sp) configuration.
Embodiment 140. The chirally enriched population of oligomeric compounds of embodiment 120, wherein each of L8 and L9 is a phosphorothioate internucleoside linkage having the (Rp) configuration and where each of L1, L2, L3, L4, L5, L6, L7, L10 and L11 is a phosphorothioate internucleoside linkage having the (Sp) configuration.
Embodiment 141. The chirally enriched population of oligomeric compounds of embodiment 120, wherein each of L9 and L10 is a phosphorothioate internucleoside linkage having the (Rp) configuration and where each of L1, L2, L3, L4, L5, L6, L7, L9 and L11 is a phosphorothioate internucleoside linkage having the (Sp) configuration.
Embodiment 142. The chirally enriched population of oligomeric compounds of embodiment 120, wherein each of L10 and L11 is a phosphorothioate internucleoside linkage having the (Rp) configuration and where each of L1, L2, L3, L4, L5, L6, L7, L8 and L9 is a phosphorothioate internucleoside linkage having the (Sp) configuration.
Embodiment 143. The chirally enriched population of oligomeric compounds of embodiment 120, wherein each L1 is a phosphorothioate internucleoside linkage having the (Rp) configuration, each L2 is a phosphorothioate internucleoside linkage having the (Sp) configuration, and where each of L3, L4, L5, L6, L7, L8, L9, L10 and L11 is a stereorandom phosphorothioate internucleoside linkage.
Embodiment 144. The chirally enriched population of oligomeric compounds of embodiment 120, wherein each L2 is a phosphorothioate internucleoside linkage having the (Rp) configuration, each L3 is a phosphorothioate internucleoside linkage having the (Sp) configuration, and where each of L1, L4, L5, L6, L7, L8, L9, L10 and L11 is a stereorandom phosphorothioate internucleoside linkage.
Embodiment 145. The chirally enriched population of oligomeric compounds of embodiment 120, wherein each L3 is a phosphorothioate internucleoside linkage having the (Rp) configuration, each L4 is a phosphorothioate internucleoside linkage having the (Sp) configuration, and where each of L1, L2, L5, L6, L7, L8, L9, L10 and L11 is a stereorandom phosphorothioate internucleoside linkage.
Embodiment 146. The chirally enriched population of oligomeric compounds of embodiment 120, wherein each L4 is a phosphorothioate internucleoside linkage having the (Rp) configuration, each L5 is a phosphorothioate internucleoside linkage having the (Sp) configuration, and where each of L1, L2, L3, L6, L7, L8, L9, L10 and L11 is a stereorandom phosphorothioate internucleoside linkage.
Embodiment 147. The chirally enriched population of oligomeric compounds of embodiment 120, wherein each L5 is a phosphorothioate internucleoside linkage having the (Rp) configuration, each L6 is a phosphorothioate internucleoside linkage having the (Sp) configuration, and wherein each of L1, L2, L3, L4, L7, L8, L9, L10 and L11 is a stereorandom phosphorothioate internucleoside linkage.
Embodiment 148. The chirally enriched population of oligomeric compounds of embodiment 120, wherein each L6 is a phosphorothioate internucleoside linkage having the (Rp) configuration, each L7 is a phosphorothioate internucleoside linkage having the (Sp) configuration, and where each of L1, L2, L3, L4, L5, L8, L9, L10 and L11 is a stereorandom phosphorothioate internucleoside linkage.
Embodiment 149. The chirally enriched population of oligomeric compounds of embodiment 120, wherein each L7 is a phosphorothioate internucleoside linkage having the (Rp) configuration, each L8 is a phosphorothioate internucleoside linkage having the (Sp) configuration, and where each of L1, L2, L3, L4, L5, L6, L9, L10 and L11 is a stereorandom phosphorothioate internucleoside linkage.
Embodiment 150. The chirally enriched population of oligomeric compounds of embodiment 120, wherein each L8 is a phosphorothioate internucleoside linkage having the (Rp) configuration, each L9 is a phosphorothioate internucleoside linkage having the (Sp) configuration, and where each of L1, L2, L3, L4, L5, L6, L7, L10 and L11 is a stereorandom phosphorothioate internucleoside linkage.
Embodiment 151. The chirally enriched population of oligomeric compounds of embodiment 120, wherein each L9 is a phosphorothioate internucleoside linkage having the (Rp) configuration, each L10 is a phosphorothioate internucleoside linkage having the (Sp) configuration, and where each of L1, L2, L3, L4, L5, L6, L7, L8 and L11 is a stereorandom phosphorothioate internucleoside linkage.
Embodiment 152. The chirally enriched population of oligomeric compounds of embodiment 120, wherein each L10 is a phosphorothioate internucleoside linkage having the (Rp) configuration, each L11 is a phosphorothioate internucleoside linkage having the (Sp) configuration, and where each of L1, L2, L3, L4, L5, L6, L7, L8 and L9 is a stereorandom phosphorothioate internucleoside linkage.
Embodiment 153. The chirally enriched population of oligomeric compounds of any of embodiments 1-152, wherein the 5′-region consists of 2-4 linked nucleosides.
Embodiment 154. The chirally enriched population of oligomeric compounds of any of embodiments 1-152, wherein the 5′-region consists of 1 nucleoside.
Embodiment 155. The chirally enriched population of oligomeric compounds of any of embodiments 1-152, wherein the 5′-region consists of 2 linked nucleosides.
Embodiment 156. The chirally enriched population of oligomeric compounds of any of embodiments 1-152, wherein the 5′-region consists of 3 linked nucleosides.
Embodiment 157. The chirally enriched population of oligomeric compounds of any of embodiments 1-152, wherein the 5′-region consists of 4 linked nucleosides.
Embodiment 158. The chirally enriched population of oligomeric compounds of any of embodiments 1-152, wherein the 5′-region consists of 5 linked nucleosides.
Embodiment 159. The chirally enriched population of oligomeric compounds of any of embodiments 1-158, wherein each nucleoside of the 5′-region comprises a 2′-modified ribosyl sugar moiety.
Embodiment 160. The chirally enriched population of oligomeric compounds of any of embodiments 1-159, wherein at least one nucleoside of the 5′-region comprises a bicyclic furanosyl sugar moiety.
Embodiment 161. The chirally enriched population of oligomeric compounds of any of embodiments 1-160, wherein each nucleoside of the 5′-region comprises a bicyclic furanosyl sugar moiety.
Embodiment 162. The chirally enriched population of oligomeric compounds of any of embodiments 1-160 wherein at least one nucleoside of the 5′-region comprises a non-bicyclic sugar moiety.
Embodiment 163. The chirally enriched population of oligomeric compounds of embodiment 162, wherein the non-bicyclic sugar moiety of the at least one nucleoside of the 5′-region is a 2′-substituted ribosyl sugar moiety.
Embodiment 164. The chirally enriched population of oligomeric compounds of any of embodiments 1-159 or 162-163, wherein each nucleoside of the 5′-region comprises a non-bicyclic sugar moiety.
Embodiment 165. The chirally enriched population of oligomeric compounds of embodiment 164, wherein each nucleoside of the 5′-region comprises a 2′-substituted ribosyl sugar moiety.
Embodiment 166. The chirally enriched population of oligomeric compounds of any of embodiments 1-159, wherein each nucleoside of the 5′-region comprises a 2′-modified furanosyl sugar moiety independently selected from a bicyclic sugar moiety and a non-bicyclic, 2′-substituted ribosyl sugar moiety.
Embodiment 167. The chirally enriched population of oligomeric compounds of any of embodiments 160-163 or 166, wherein each bicyclic sugar moiety of the 5′-region is selected from among cEt, LNA, and ENA.
Embodiment 168. The chirally enriched population of oligomeric compounds of any of embodiments 162-166, wherein each non-bicyclic sugar moiety of the 5′-region has a 2′-substituent selected from among 2′-MOE, 2′-OMe, and 2′-NMA.
Embodiment 169. The chirally enriched population of oligomeric compounds of any of embodiments 1-168, wherein none of the nucleosides of the 5′-region comprise a sugar moiety having a 2′-F substituent.
Embodiment 170. The chirally enriched population of oligomeric compounds of any of embodiments 1-169, wherein each nucleobase of the 5′-region is independently selected from among thymine, uracil, guanine, cytosine, 5-methylcytosine, and adenine.
Embodiment 171. The chirally enriched population of oligomeric compounds of any of embodiments 1-170, wherein each internucleoside linkage of the 5′-region is selected from among phosphodiester and phosphorothioate internucleoside linkages.
Embodiment 172. The chirally enriched population of oligomeric compounds of any of embodiments 1-171, wherein the 3′-region consists of 2-4 linked nucleosides.
Embodiment 173. The chirally enriched population of oligomeric compounds of any of embodiments 1-171, wherein the 3′-region consists of 1 nucleoside.
Embodiment 174. The chirally enriched population of oligomeric compounds of any of embodiments 1-171, wherein the 3′-region consists of 2 linked nucleosides.
Embodiment 175. The chirally enriched population of oligomeric compounds of any of embodiments 1-171, wherein the 3′-region consists of 3 linked nucleosides.
Embodiment 176. The chirally enriched population of oligomeric compounds of any of embodiments 1-170, wherein the 3′-region consists of 4 linked nucleosides.
Embodiment 177. The chirally enriched population of oligomeric compounds of any of embodiments 1-171, wherein the 3′-region consists of 5 linked nucleosides.
Embodiment 178. The chirally enriched population of oligomeric compounds of any of embodiments 1-177, wherein each nucleoside of the 3′-region comprises a 2′-modified ribosyl sugar moiety.
Embodiment 179. The chirally enriched population of oligomeric compounds of any of embodiments 1-178, wherein at least one nucleoside of the 3′-region comprises a bicyclic furanosyl sugar moiety.
Embodiment 180. The chirally enriched population of oligomeric compounds of any of embodiments 1-179, wherein each nucleoside of the 3′-region comprises a bicyclic furanosyl sugar moiety.
Embodiment 181. The chirally enriched population of oligomeric compounds of any of embodiments 1-179, wherein at least one nucleoside of the 3′-region comprises a non-bicyclic sugar moiety.
Embodiment 182. The chirally enriched population of oligomeric compounds of embodiment 1-181, wherein the non-bicyclic sugar moiety of the at least one nucleoside of the 3′-region is a 2′-substituted ribosyl sugar moiety.
Embodiment 183. The chirally enriched population of oligomeric compounds of any of embodiments 1-178 or 181-182, wherein each nucleoside of the 3′-region comprises a non-bicyclic sugar moiety.
Embodiment 184. The chirally enriched population of oligomeric compounds of embodiment 183, wherein each nucleoside of the 3′-region comprises a 2′-substituted ribosyl sugar moiety.
Embodiment 185. The chirally enriched population of oligomeric compounds of any of embodiments 1-178, wherein each nucleoside of the 3′-region comprises a 2′-modified furanosyl sugar moiety independently selected from a bicyclic sugar moiety and a non-bicyclic, 2′-substituted ribosyl sugar moiety.
Embodiment 186. The chirally enriched population of oligomeric compounds of any of embodiments 179-182 or 185, wherein each bicyclic sugar moiety of the 3′-region is selected from among cEt, LNA, and ENA.
Embodiment 187. The chirally enriched population of oligomeric compounds of any of embodiments 181-185, wherein each non-bicyclic sugar moiety of the 3′-region has a 2′-substituent selected from among 2′-MOE, 2′-OMe, and 2′-NMA.
Embodiment 188. The chirally enriched population of oligomeric compounds of any of embodiments 1-187, wherein none of the nucleosides of the 3′-region comprise a sugar moiety having a 2′-F substituent.
Embodiment 189. A pharmaceutical composition comprising, the chirally enriched population of oligomeric compounds of any of embodiments 1-187.
Embodiment 190. A method comprising, contacting a cell with the chirally enriched population of oligomeric compounds of any of embodiments 1-187.
Embodiment 191. A method of modulating the amount or activity of a target nucleic acid in a cell, comprising contacting a cell with the chirally enriched population of oligomeric compounds of any of embodiments 1-187.

### I. Modifications

### A. Modified Nucleosides

Modified nucleosides comprise a modified sugar moiety, a modified nucleobase, or both a modifed sugar moiety and a modified nucleobase.

1. Certain Modified Sugar Moieties

In certain embodiments, sugar moieties are non-bicyclic, modified furanosyl sugar moieties. In certain embodiments, modified sugar moieties are bicyclic or tricyclic furanosyl sugar moieties. In certain embodiments, modified sugar moieties are sugar surrogates. Such sugar surrogates may comprise one or more substitutions corresponding to those of other types of modified sugar moieties.

In certain embodiments, modified sugar moieties are non-bicyclic modified furanosyl sugar moieties comprising one or more acyclic substituent, including but not limited to substituents at the 2′, 3′, 4′, and/or 5′ positions. In certain embodiments, the furanosyl sugar moiety is a ribosyl sugar moiety. In certain embodiments one or more acyclic substituent of non-bicyclic modified sugar moieties is branched. Examples of 2′-substituent groups suitable for non-bicyclic modified sugar moieties include but are not limited to: 2′-F, 2′-OCH₃(“OMe” or “O-methyl”), and 2′-O(CH₂)₂OCH₃ (“MOE”). In certain embodiments, 2′-substituent groups are selected from among: halo, allyl, amino, azido, SH, CN, OCN, CF₃, OCF₃, O-C₁-C₁₀ alkoxy, O—C₁-C₁₀ substituted alkoxy, O-C₁-C₁₀ alkyl, O-C₁-C₁₀ substituted alkyl, S-alkyl, N(Rₘ)-alkyl, O-alkenyl, S-alkenyl, N(Rₘ)-alkenyl, O-alkynyl, S-alkynyl, N(Rₘ)-alkynyl, O-alkylenyl-O-alkyl, alkynyl, alkaryl, aralkyl, O-alkaryl, O-aralkyl, O(CH₂)₂SCH₃, O(CH₂)₂ON(Rₘ)(Rₙ) or OCH₂C(═O)-N(Rₘ)(Rₙ), where each Rₘ and Rₙ is, independently, H, an amino protecting group, or substituted or unsubstituted C₁-C₁₀ alkyl, and the 2′-substituent groups described in Cook et al., U.S. Pat. No. 6,531,584; Cook et al., U.S. Pat. No. 5,859,221; and Cook et al., U.S. Pat. No. 6,005,087. Certain embodiments of these 2′-substituent groups can be further substituted with one or more substituent groups independently selected from among: hydroxyl, amino, alkoxy, carboxy, benzyl, phenyl, nitro (NO2), thiol, thioalkoxy, thioalkyl, halogen, alkyl, aryl, alkenyl and alkynyl. Examples of 4′-substituent groups suitable for non-bicyclic modified sugar moieties include but are not limited to alkoxy (e.g., methoxy), alkyl, and those described in Manoharan et al., WO 2015/106128. Examples of 5′-substituent groups suitable for non-bicyclic modified sugar moieties include but are not limited to: 5′-methyl (R or S), 5′-vinyl, and 5′-methoxy. In certain embodiments, non-bicyclic modified sugars comprise more than one non-bridging sugar substituent, for example, 2′-F-5′-methyl sugar moieties and the modified sugar moieties and modified nucleosides described in Migawa et al., WO 2008/101157 and Rajeev et al., US2013/0203836.

In certain embodiments, a 2′-substituted nucleoside or non-bicyclic 2′-modified nucleoside comprises a sugar moiety comprising a non-bridging 2′-substituent group selected from: F, NH₂, N₃, OCF₃, OCH₃, O(CH₂)₃NH₂, CH₂CH═CH₂, OCH₂CH═CH₂, OCH₂CH₂OCH₃, O(CH₂)₂SCH₃, O(CH₂)₂ON(Rₘ)(Rₙ), O(CH₂)₂O(CH₂)₂N(CH₃)₂, and N-substituted acetamide (OCH₂C(═O)-N(Rₘ)(Rₙ)), where each Rₘ and Rₙ is, independently, H, an amino protecting group, or substituted or unsubstituted C₁-C₁₀ alkyl.

In certain embodiments, a 2′-substituted nucleoside or non-bicyclic 2′-modified nucleoside comprises a sugar moiety comprising a non-bridging 2′-substituent group selected from: F, OCF₃, OCH₃, OCH₂CH₂OCH₃, O(CH₂)₂SCH₃, O(CH₂)₂ON(CH₃)₂, O(CH₂)₂O(CH₂)₂N(CH₃)₂, and OCH₂C(═O)-N(H)CH₃ (“NMA”).

In certain embodiments, a 2′-substituted nucleoside or non-bicyclic 2′-modified nucleoside comprises a sugar moiety comprising a non-bridging 2′-substituent group selected from: F, OCH₃, and OCH₂CH₂OCH₃.

Certain modifed sugar moieties comprise a bridging sugar substituent that forms a second ring resulting in a bicyclic sugar moiety. In certain such embodiments, the bicyclic sugar moiety comprises a bridge between the 4′ and the 2′ furanose ring atoms. In certain such embodiments, the furanose ring is a ribose ring. Examples of sugar moieties comprising such 4′ to 2′ bridging sugar substituents include but are not limited to bicyclic sugars comprising: 4′-CH₂-2′, 4′-(CH₂)₂-2′, 4′-(CH₂)₃-2′, 4′-CH₂-O-2′ (“LNA”), 4′-CH₂—S-2′, 4′-(CH₂)₂-O-2′ (“ENA”), 4′-CH(CH₃)-O-2′ (referred to as “constrained ethyl” or “cEt” when in the S configuration), 4′-CH₂-O—CH₂-2′, 4′-CH₂-N(R)-2′, 4′-CH(CH₂OCH₃)-O-2′ (“constrained MOE” or “cMOE”) and analogs thereof (see, e.g., Seth et al., U.S. Pat. No. 7,399,845, Bhat et al., U.S. Pat. No. 7,569,686, Swayze et al., U.S. Pat. No. 7,741,457, and Swayze et al., U.S. Pat. No. 8,022,193), 4′-C(CH₃)(CH₃)-O-2′ and analogs thereof (see, e.g., Seth et al., U.S. Pat. No. 8,278,283), 4′-CH₂-N(OCH3)-2′ and analogs thereof (see, e.g., Prakash et al., U.S. Pat. No. 8,278,425), 4′-CH₂-O—N(CH₃)-2′ (see, e.g., Allerson et al., U.S. Pat. No. 7,696,345 and Allerson et al., U.S. Pat. No. 8,124,745), 4′-CH₂-C(H)(CH₃)-2′ (see, e.g., Zhou, et al., J. Org. Chem.,2009, 74, 118-134), 4′-CH₂-C(═CH₂)-2′ and analogs thereof (see e.g., Seth et al., U.S. Pat. No. 8,278,426), 4′-C(RₐR_{b})-N(R)-O-2′, 4′-C(RₐR_{b})-O—N(R)-2′, 4′-CH₂-O—N(R)-2′, and 4′-CH₂—N(R)-O-2′, wherein each R, Rₐ, and R_{b} is, independently, H, a protecting group, or C₁-C₁₂ alkyl (see, e.g. Imanishi et al., U.S. Pat. No. 7,427,672).

In certain embodiments, such 4′ to 2′ bridges independently comprise from 1 to 4 linked groups independently selected from: —[C(Rₐ)(R_{b})]ₙ—, —[C(Rₐ)(R_{b})]ₙ—O—, —C(Rₐ)═C(R_{b})—, —C(Rₐ)═N—, —C(═NRₐ)—, —C(═O)—, —C(═S)—, —O—, —Si(Rₐ)₂—, —S(═O)ₓ—, and —N(Rₐ)—;

wherein:

x is 0, 1, or 2;

n is 1, 2, 3, or 4;

each Rₐ and R_{b}, is, independently, H, a protecting group, hydroxyl, C₁-C12 alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and

each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl, or a protecting group.

Additional bicyclic sugar moieties are known in the art, see, for example: Freier et al., Nucleic Acids Research, 1997, 25(22), 4429-4443, Albaek et al., J. Org. Chem., 2006, 71, 7731-7740, Singh et al., Chem. Commun., 1998, 4, 455-456; Koshkin et al., Tetrahedron, 1998, 54, 3607-3630; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 20017, 129, 8362-8379; Elayadi et al.,; Wengel et a., U.S. 7,053,207; Imanishi et al., U.S. Pat. No. 6,268,490; Imanishi et al. U.S. Pat. No. 6,770,748; Imanishi et al., U.S. RE44,779; Wengel et al., U.S. Pat. No. 6,794,499; Wengel et al., U.S. Pat. No. 6,670,461; Wengel et al., U.S. Pat. No. 7,034,133; Wengel et al., U.S. Pat. No. 8,080,644; Wengel et al., U.S. Pat. No. 8,034,909; Wengel et al., U.S. Pat. No. 8,153,365; Wengel et al., U.S. Pat. No. 7,572,582; and Ramasamy et al., U.S. Pat. No. 6,525,191; Torsten et al., WO 2004/106356;Wengel et al., WO 1999/014226; Seth et al., WO 2007/134181; Seth et al., U.S. Pat. No. 7,547,684; Seth et al., U.S. Pat. No. 7,666,854; Seth et al., U.S. Pat. No. 8,088,746; Seth et al., U.S. Pat. No. 7,750,131; Seth et al., U.S. Pat. No. 8,030,467; Seth et al., U.S. Pat. No. 8,268,980; Seth et al., U.S. Pat. No. 8,546,556; Seth et al., U.S. Pat. No. 8,530,640; Migawa et al., U.S. Pat. No. 9,012,421; Seth et al., U.S. Pat. No. 8,501,805; and U.S. Patent Publication Nos. Allerson et al., US2008/0039618 and Migawa et al., US2015/0191727.

In certain embodiments, bicyclic sugar moieties and nucleosides incorporating such bicyclic sugar moieties are further defined by isomeric configuration. For example, an LNA nucleoside (described herein) may be in the α-L configuration or in the β-D configuration.

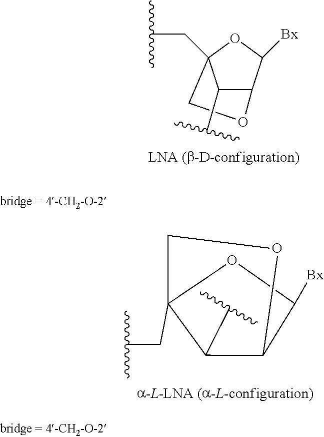

α-L-methyleneoxy (4′-CH₂—O—2′) or α-L-LNA bicyclic nucleosides have been incorporated into antisense oligonucleotides that showed antisense activity (Frieden et al., Nucleic Acids Research, 2003, 21, 6365-6372). Herein, general descriptions of bicyclic nucleosides include both isomeric configurations. When the positions of specific bicyclic nucleosides (e.g., LNA) are identified in exemplified embodiments herein, they are in the β-D configuration, unless otherwise specified.

In certain embodiments, modified sugar moieties comprise one or more non-bridging sugar substituent and one or more bridging sugar substituent (e.g., 5′-substituted and 4′-2′ bridged sugars).

Nucleosides comprising modified furanosyl sugar moieties and modified furanosyl sugar moieties may be referred to by the position(s) of the substitution(s) on the sugar moiety of the nucleoside. The term “modified” following a position of the furanosyl ring, such as “2′-modified”, indicates that the sugar moiety comprises the indicated modification at the 2′ position and may comprise additional modifications and/or substituents. The term “substituted” following a position of the furanosyl ring, such as “2′-substituted” or “2′-4′-substituted”, indicates that is the only position(s) having a substituent other than those found in unmodified sugar moieties in oligonucleotides. Accordingly, the following sugar moieties are represented by the following formulas.

In the context of a nucleoside and/or an oligonucleotide, a non-bicyclic, modified furanosyl sugar moiety is represented by formula I:

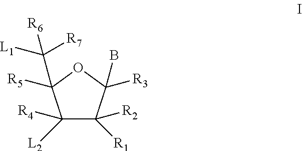

wherein B is a nucleobase; and L₁ and L₂ are each, independently, an internucleoside linkage, a terminal group, a conjugate group, or a hydroxyl group. Among the R groups, at least one of R₃₋₇ is not H and/or at least one of R₁ and R₂ is not H or OH. In a 2′-modified furanosyl sugar moiety, at least one of R₁ and R₂ is not H or OH and each of R₃₋₇ is independently selected from H or a substituent other than H. In a 4′-modified furanosyl sugar moiety, R₅ is not H and each of R_{1-4, 6, 7} are independently selected from H and a substituent other than H; and so on for each position of the furanosyl ring. The stereochemistry is not defined unless otherwise noted.

In the context of a nucleoside and/or an oligonucleotide, a non-bicyclic, modified, substituted fuarnosyl sugar moiety is represented by formula I, wherein B is a nucleobase; and L1 and L2 are each, independently, an internucleoside linkage, a terminal group, a conjugate group, or a hydroxyl group. Among the R groups, either one (and no more than one) of R₃₋₇ is a substituent other than H or one of R₁ or R₂ is a substituent other than H or OH. The stereochemistry is not defined unless otherwise noted. Examples of non-bicyclic, modified, substituted furanosyl sugar moieties include 2′-substituted ribosyl, 4′-substituted ribosyl, and 5′-substituted ribosyl sugar moieties, as well as substituted 2′-deoxyfuranosyl sugar moieties, such as 4′-substituted 2′-deoxyribosyl and 5′-substituted 2′-deoxyribosyl sugar moieties.

In the context of a nucleoside and/or an oligonucleotide, a 2′-substituted ribosyl sugar moiety is represented by formula II:

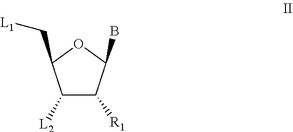

wherein B is a nucleobase; and L₁ and L₂ are each, independently, an internucleoside linkage, a terminal group, a conjugate group, or a hydroxyl group. R₁ is a substituent other than H or OH. The stereochemistry is defined as shown.

In the context of a nucleoside and/or an oligonucleotide, a 4′-substituted ribosyl sugar moiety is represented by formula III:

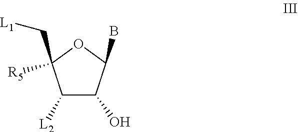

wherein B is a nucleobase; and L₁ and L₂ are each, independently, an internucleoside linkage, a terminal group, a conjugate group, or a hydroxyl group. R₅ is a substituent other than H. The stereochemistry is defined as shown.

In the context of a nucleoside and/or an oligonucleotide, a 5′-substituted ribosyl sugar moiety is represented by formula IV:

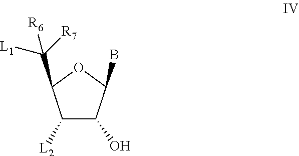

wherein B is a nucleobase; and L₁ and L₂ are each, independently, an internucleoside linkage, a terminal group, a conjugate group, or a hydroxyl group. R₆ or R₇ is a substituent other than H. The stereochemistry is defined as shown.

In the context of a nucleoside and/or an oligonucleotide, a 2′-deoxyfuranosyl sugar moiety is represented by formula V:

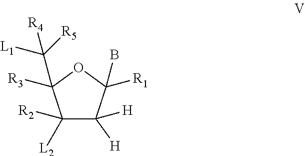

wherein B is a nucleobase; and L₁ and L₂ are each, independently, an internucleoside linkage, a terminal group, a conjugate group, or a hydroxyl group. Each of R₁₋₅ are indepently selected from H and a non-H substituent. If all of R₁₋₅ are each H, the sugar moiety is an unsubstituted 2′-deoxyfuranosyl sugar moiety. The stereochemistry is not defined unless otherwise noted.

In the context of a nucleoside and/or an oligonucleotide, a 4′-substituted 2′-deoxyribosyl sugar moiety is represented by formula VI:

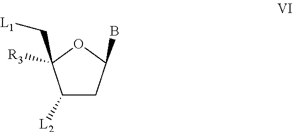

wherein B is a nucleobase; and L₁ and L₂ are each, independently, an internucleoside linkage, a terminal group, a conjugate group, or a hydroxyl group. R₃ is a substituent other than H. The stereochemistry is defined as shown.

In the context of a nucleoside and/or an oligonucleotide, a 5′-substituted 2′-deoxyribosyl sugar moiety is represented by formula VII:

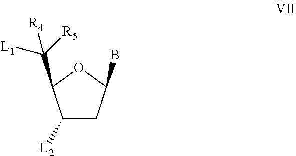

wherein B is a nucleobase; and L₁ and L₂ are each, independently, an internucleoside linkage, a terminal group, a conjugate group, or a hydroxyl group. R₄ or R₅ is a substituent other than H. The stereochemistry is defined as shown.

Unsubstituted 2′-deoxyfuranosyl sugar moieties may be unmodified (β-D-2′-deoxyribosyl) or modified. Examples of modified, unsubstituted 2′-deoxyfuranosyl sugar moieties include β-L-2′-deoxyribosyl, α-L-2′-deoxyribosyl, α-D-2′-deoxyribosyl, and β-D-xylosyl sugar moieties. For example, in the context of a nucleoside and/or an oligonucleotide, a β-L-2′-deoxyribosyl sugar moiety is represented by formula VIII:

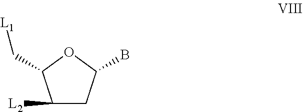

wherein B is a nucleobase; and L₁ and L₂ are each, independently, an internucleoside linkage, a terminal group, a conjugate group, or a hydroxyl group. The stereochemistry is defined as shown.

In certain embodiments, modified sugar moieties are sugar surrogates. In certain such embodiments, the oxygen atom of the sugar moiety is replaced, e.g., with a sulfur, carbon or nitrogen atom. In certain such embodiments, such modified sugar moieties also comprise bridging and/or non-bridging substituents as described herein. For example, certain sugar surrogates comprise a 4′-sulfur atom and a substitution at the 2′-position (see, e.g., Bhat et al., U.S. Pat. No. 7,875,733 and Bhat et al., U.S. Pat. No. 7,939,677) and/or the 5′ position.

In certain embodiments, sugar surrogates comprise rings having other than 5 atoms. For example, in certain embodiments, a sugar surrogate comprises a six-membered tetrahydropyran (“THP”). Such tetrahydropyrans may be further modified or substituted. Nucleosides comprising such modified tetrahydropyrans include but are not limited to hexitol nucleic acid (“HNA”), altritol nucleic acid (“ANA”), mannitol nucleic acid (“MNA”) (see, e.g., Leumann, C J. Bioorg. & Med. Chem. 2002, 10, 841-854), fluoro HNA:

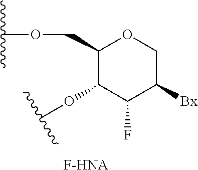

(“F-HNA”, see e.g. Swayze et al., U.S. Pat. No. 8,088,904; Swayze et al., U.S. Pat. No. 8,440,803; Swayze et al., U.S. Pat. No. 8,796,437; and Swayze et al., U.S. Pat. No. 9,005,906; F-HNA can also be referred to as a F-THP or 3′-fluoro tetrahydropyran), and nucleosides comprising additional modified THP compounds having the formula:

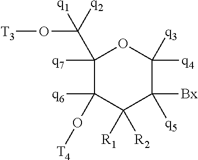

wherein, independently, for each of said modified THP nucleoside:

Bx is a nucleobase moiety;

T₃ and T₄ are each, independently, an internucleoside linkage linking the modified THP nucleoside to the remainder of an oligonucleotide or one of T₃ and T₄ is an internucleoside linkage linking the modified THP nucleoside to the remainder of an oligonucleotide and the other of T₃ and T₄ is H, a hydroxyl protecting group, a linked conjugate group, or a 5′ or 3′-terminal group;
q₁, q₂, q₃, q₄, q₅, q₆ and q₇ are each, independently, H, C₁-C₆ alkyl, substituted C₁-C₆ alkyl, C₂-C₆ alkenyl, substituted C₂-C₆ alkenyl, C₂-C₆ alkynyl, or substituted C₂-C₆ alkynyl; and

each of R₁ and R₂ is independently selected from among: hydrogen, halogen, substituted or unsubstituted alkoxy, NJ₁J₂, SJ₁, N₃, OC(═X)J₁, OC(═X)NJ₁J₂, NJ₃C(═X)NJ₁J₂, and CN, wherein X is O, S or NJ₁, and each J₁, J₂, and J₃ is, independently, H or C₁-C₆ alkyl.

In certain embodiments, modified THP nucleosides are provided wherein q₁, q₂, q₃, q₄, q₅, q₆ and q₇ are each H. In certain embodiments, at least one of q₁, q₂, q₃, q₄, q₅, q₆ and q₇ is other than H. In certain embodiments, at least one of q₁, q₂, q₃, q₄, q₅, q₆ and q₇ is methyl. In certain embodiments, modified THP nucleosides are provided wherein one of R₁ and R₂ is F. In certain embodiments, R₁ is F and R₂ is H, in certain embodiments, R₁ is methoxy and R₂ is H, and in certain embodiments, R₁ is methoxyethoxy and R₂ is H.

In certain embodiments, sugar surrogates comprise rings having more than 5 atoms and more than one heteroatom. For example, nucleosides comprising morpholino sugar moieties and their use in oligonucleotides have been reported (see, e.g., Braasch et al., Biochemistry, 2002, 41, 4503-4510 and Summerton et al., U.S. Pat. No. 5,698,685; Summerton et al., U.S. Pat. No. 5,166,315; Summerton et al., U.S. Pat. No. 5,185,444; and Summerton et al., U.S. Pat. No. 5,034,506). As used here, the term “morpholino” means a sugar surrogate having the following structure:

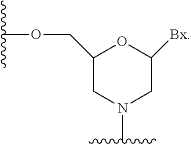

In certain embodiments, morpholinos may be modified, for example by adding or altering various substituent groups from the above morpholino structure. Such sugar surrogates are refered to herein as “modifed morpholinos.”

In certain embodiments, sugar surrogates comprise acyclic moieites. Examples of nucleosides and oligonucleotides comprising such acyclic sugar surrogates include but are not limited to: peptide nucleic acid (“PNA”), acyclic butyl nucleic acid (see, e.g., Kumar et al., Org. Biomol. Chem., 2013, 11, 5853-5865), and nucleosides and oligonucleotides described in Manoharan et al., WO2011/133876.

Many other bicyclic and tricyclic sugar and sugar surrogate ring systems are known in the art that can be used in modified nucleosides.

In certain embodiments, modified nucleosides are DNA mimics. In certain embodiments, a DNA mimic is a sugar surrogate. In certain embodiments, a DNA mimic is a cycohexenyl or hexitol nucleic acid. In certain embodiments, a DNA mimic is described in Figure 1 of Vester, et. al., “Chemically modified oligonucleotides with efficient RNase H response,” Bioorg. Med. Chem. Letters, 2008, 18: 2296-2300, incorporated by reference herein. In certain embodiments, a DNA mimic nucleoside has a formula selected from:

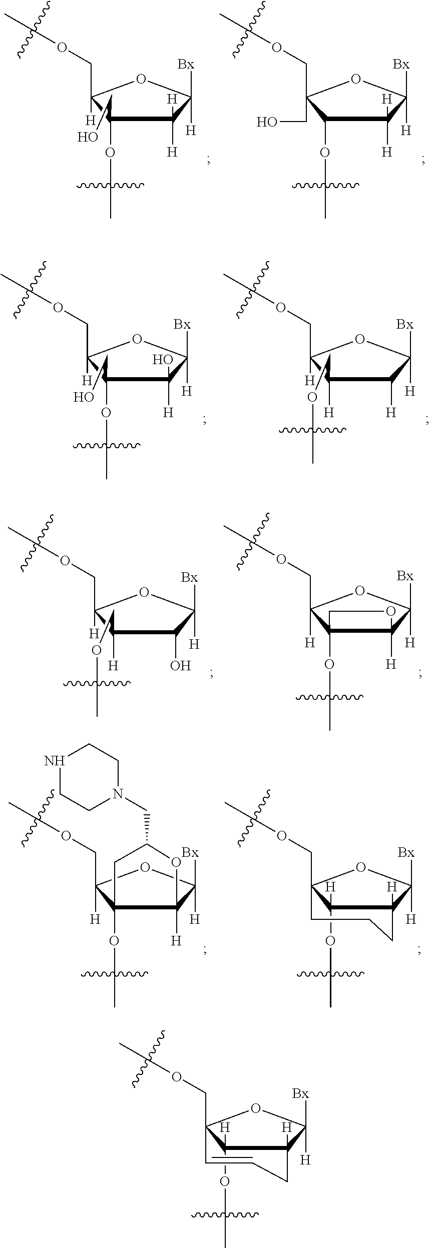

wherein Bx is a heterocyclic base moiety. In certain embodiments, a DNA mimic is α,β-constrained nucleic acid (CAN), 2′,4′-carbocyclic-LNA, or 2′,4′-carbocyclic-ENA. In certain embodiments, a DNA mimic has a sugar moiety selected from among: 4′-C-hydroxymethyl-2′-deoxyribosyl, 3′-C-hydroxymethyl-2′-deoxyribosyl, 3′-C-hydroxymethyl-arabinosyl, 3′-C-2′-O-arabinosyl, 3′-C-methylene-extended-2′-deoxyxylosyl, 3′-C-methylene-extended-xyolosyl, 3′-C-2′-0-piperazino-arabinosyl. In certain embodiments, a DNA mimic has a sugar moiety selected from among: 2′-methylribosyl, 2′-S-methylribosyl, 2′-aminoribosyl, 2′-NH(CH2)-ribosyl, 2′-NH(CH2)2-ribosyl, 2′-CH2-F-ribosyl, 2′-CHF2-ribosyl, 2′-CF3-ribosyl, 2′=CF2 ribosyl, 2′-ethylribosyl, 2′-alkenylribosyl, 2′-alkynylribosyl, 2′-O-4′-C-methyleneribosyl, 2′-cyanoarabinosyl, 2′-chloroarabinosyl, 2′-fluoroarabinosyl, 2′-bromoarabinosyl, 2′-azidoarabinosyl, 2′-methoxyarabinosyl, and 2′-arabinosyl. In certain embodiments, a DNA mimic has a sugar moiety selected from 4′-methyl-modified deoxyfuranosyl, 4′-F-deoxyfuranosyl, 4′-OMe-deoxyfuranosyl. In certain embodiments, a DNA mimic has a sugar moiety selected from among: 5′-methyl-2′-β-D-deoxyribosyl, 5′-ethyl-2′-β-D-deoxyribosyl, 5′-allyl-2′-β-D-deoxyribosyl, 2′-fluoro-β-D-arabinofuranosyl. In certain embodiments, DNA mimics are listed on page 32-33 of PCT/US00/267929 as B-form nucleotides, incorporated by reference herein in its entirety.

2. Modified Nucleobases

In certain embodiments, modified nucleobases are selected from: 5-substituted pyrimidines, 6-azapyrimidines, alkyl or alkynyl substituted pyrimidines, alkyl substituted purines, and N-2, N-6 and O-6 substituted purines. In certain embodiments, modified nucleobases are selected from: 2-aminopropyladenine, 5-hydroxymethyl cytosine, xanthine, hypoxanthine, 2-aminoadenine, 6-N-methylguanine, 6-N-methyladenine, 2-propyladenine , 2-thiouracil, 2-thiothymine and 2-thiocytosine, 5-propynyl (—C≡C—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in Merigan et al., U.S. Pat. No. 3,687,808, those disclosed in The Concise Encyclopedia Of Polymer Science And Engineering, Kroschwitz, J. I., Ed., John Wiley & Sons, 1990, 858-859; Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443.

Publications that teach the preparation of certain of the above noted modified nucleobases as well as other modified nucleobases include without limitation, Manoharan et al., US2003/0158403; Manoharan et al., US2003/0175906; Dinh et al., U.S. Pat. No. 4,845,205; Spielvogel et al., U.S. Pat. No. 5,130,302; Rogers et al., U.S. Pat. No. 5,134,066; Bischofberger et al., U.S. Pat. No. 5,175,273; Urdea et al., U.S. Pat. No. 5,367,066; Benner et al., U.S. Pat. No. 5,432,272; Matteucci et al., U.S. Pat. No. 5,434,257; Gmeiner et al., U.S. Pat. No. 5,457,187; Cook et al., U.S. Pat. No. 5,459,255; Froehler et al., U.S. Pat. No. 5,484,908; Matteucci et al., U.S. Pat. No. 5,502,177; Hawkins et al., U.S. Pat. No. 5,525,711; Haralambidis et al., U.S. Pat. No. 5,552,540; Cook et al., U.S. Pat. No. 5,587,469; Froehler et al., U.S. Pat. No. 5,594,121; Switzer et al., U.S. Pat. No. 5,596,091; Cook et al., U.S. Pat. No. 5,614,617; Froehler et al., U.S. Pat. No. 5,645,985; Cook et al., U.S. Pat. No. 5,681,941; Cook et al., U.S. Pat. No. 5,811,534; Cook et al., U.S. Pat. No. 5,750,692; Cook et al., U.S. Pat. No. 5,948,903; Cook et al., U.S. Pat. No. 5,587,470; Cook et al., U.S. Pat. No. 5,457,191; Matteucci et al., U.S. Pat. No. 5,763,588; Froehler et al., U.S. Pat. No. 5,830,653; Cook et al., U.S. Pat. No. 5,808,027; Cook et al., 6,166,199; and Matteucci et al., U.S. Pat. No. 6,005,096.

In certain embodiments, compounds comprise or consist of a modified oligonucleotide complementary to an target nucleic acid comprising one or more modified nucleobases. In certain embodiments, the modified nucleobase is 5-methylcytosine. In certain embodiments, each cytosine is a 5-methylcyto sine.

### B. Modified Internucleoside Linkages

In certain embodiments, compounds described herein having one or more modified internucleoside linkages are selected over compounds having only phosphodiester internucleoside linkages because of desirable properties such as, for example, enhanced cellular uptake, enhanced affinity for target nucleic acids, and increased stability in the presence of nucleases.

In certain embodiments, compounds comprise or consist of a modified oligonucleotide complementary to a target nucleic acid comprising one or more modified internucleoside linkages. In certain embodiments, the modified internucleoside linkages are phosphorothioate linkages. In certain embodiments, each internucleoside linkage of an antisense compound is a phosphorothioate internucleoside linkage.

In certain embodiments, nucleosides of modified oligonucleotides may be linked together using any internucleoside linkage. The two main classes of internucleoside linkages are defined by the presence or absence of a phosphorus atom. Representative phosphorus-containing internucleoside linkages include unmodified phosphodiester internucleoside linkages, modified phosphotriesters such as THP phosphotriester and isopropyl phosphotriester, phosphonates such as methylphosphonate, isopropyl phosphonate, isobutyl phosphonate, and phosphonoacetate, phosphoramidates, phosphorothioate, and phosphorodithioate (“HS—P═S”). Representative non-phosphorus containing internucleoside linkages include but are not limited to methylenemethylimino (—CH₂—N(CH₃)—O—CH2—), thiodiester, thionocarbamate (—O—C(═O)(NH)—S—); siloxane (—O—SiH₂—O—); formacetal, thioacetamido (TANA), alt-thioformacetal, glycine amide, and N,N′-dimethylhydrazine (—CH₂—N(CH₃)—N(CH₃)—). Modified internucleoside linkages, compared to naturally occurring phosphate linkages, can be used to alter, typically increase, nuclease resistance of the oligonucleotide. Methods of preparation of phosphorous-containing and non-phosphorous-containing internucleoside linkages are well known to those skilled in the art.

Representative internucleoside linkages having a chiral center include but are not limited to alkylphosphonates and phosphorothioates. Modified oligonucleotides comprising internucleoside linkages having a chiral center can be prepared as populations of modified oligonucleotides comprising stereorandom internucleoside linkages, or as populations of modified oligonucleotides comprising phosphorothioate linkages in particular stereochemical configurations. In certain embodiments, populations of modified oligonucleotides comprise phosphorothioate internucleoside linkages wherein all of the phosphorothioate internucleoside linkages are stereorandom. Such modified oligonucleotides can be generated using synthetic methods that result in random selection of the stereochemical configuration of each phosphorothioate linkage. Nonetheless, as is well understood by those of skill in the art, each individual phosphorothioate of each individual oligonucleotide molecule has a defined stereoconfiguration. In certain embodiments, populations of modified oligonucleotides are enriched for modified oligonucleotides comprising one or more particular phosphorothioate internucleoside linkages in a particular, independently selected stereochemical configuration. In certain embodiments, the particular configuration of the particular phosphorothioate linkage is present in at least 65% of the molecules in the population. In certain embodiments, the particular configuration of the particular phosphorothioate linkage is present in at least 70% of the molecules in the population. In certain embodiments, the particular configuration of the particular phosphorothioate linkage is present in at least 80% of the molecules in the population. In certain embodiments, the particular configuration of the particular phosphorothioate linkage is present in at least 90% of the molecules in the population. In certain embodiments, the particular configuration of the particular phosphorothioate linkage is present in at least 99% of the molecules in the population. Such chirally enriched populations of modified oligonucleotides can be generated using synthetic methods known in the art, e.g., methods described in Oka et al., JACS 125, 8307 (2003), Wan et al. Nuc. Acid. Res. 42, 13456 (2014), and WO 2017/015555. In certain embodiments, a population of modified oligonucleotides is enriched for modified oligonucleotides having at least one indicated phosphorothioate in the (Sp) configuration. In certain embodiments, a population of modified oligonucleotides is enriched for modified oligonucleotides having at least one phosphorothioate in the (Rp) configuration. In certain embodiments, modified oligonucleotides comprising (Rp) and/or (Sp) phosphorothioates comprise one or more of the following formulas, respectively, wherein “B” indicates a nucleobase:

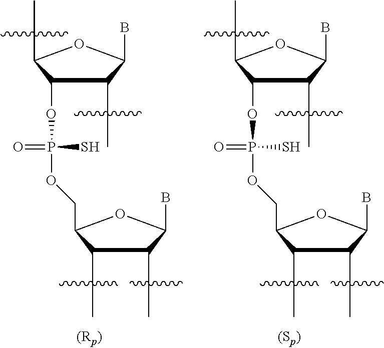

Unless otherwise indicated, chiral internucleoside linkages of modified oligonucleotides described herein can be stereorandom or in a particular stereochemical configuration.

Neutral internucleoside linkages include, without limitation, phosphotriesters, phosphonates, MMI (3′-CH₂—N(CH₃)—O-5′), amide-3 (3′-CH₂—C(═O)—N(H)-5′), amide-4 (3′-CH₂—N(H)—C(═O)-5′), formacetal (3′-O—CH₂-O-5), methoxypropyl, and thioformacetal (3′-S—CH₂—O-5′). Further neutral internucleoside linkages include nonionic linkages comprising siloxane (dialkylsiloxane), carboxylate ester, carboxamide, sulfide, sulfonate ester and amides (See for example: Carbohydrate Modifications in Antisense Research; Y. S. Sanghvi and P.D. Cook, Eds., ACS Symposium Series 580; Chapters 3 and 4, 40-65). Further neutral internucleoside linkages include nonionic linkages comprising mixed N, O, S and CH2 component parts.

II. Certain Motifs

In certain embodiments, oligomeric compounds described herein comprise or consist of oligonucleotides. Oligonucleotides can have a motif, e.g. a pattern of unmodified and/or modified sugar moieties, nucleobases, and/or internucleoside linkages. In certain embodiments, modified oligonucleotides comprise one or more modified nucleoside comprising a modified sugar. In certain embodiments, modified oligonucleotides comprise one or more modified nucleosides comprising a modified nucleobase. In certain embodiments, modified oligonucleotides comprise one or more modified internucleoside linkage. In such embodiments, the modified, unmodified, and differently modified sugar moieties, nucleobases, and/or internucleoside linkages of a modified oligonucleotide define a pattern or motif. In certain embodiments, the patterns or motifs of sugar moieties, nucleobases, and internucleoside linkages are each independent of one another. Thus, a modified oligonucleotide may be described by its sugar motif, nucleobase motif and/or internucleoside linkage motif (as used herein, nucleobase motif describes the modifications to the nucleobases independent of the sequence of nucleobases).

A. Certain Sugar Motifs

In certain embodiments, oligomeric compounds described herein comprise or consist of oligonucleotides. In certain embodiments, oligonucleotides comprise one or more type of modified sugar and/or unmodified sugar moiety arranged along the oligonucleotide or region thereof in a defined pattern or sugar motif. In certain instances, such sugar motifs include but are not limited to any of the sugar modifications discussed herein.

In certain embodiments, a modified oligonucleotide comprises or has a uniformly modified sugar motif. An oligonucleotide comprising a uniformly modified sugar motif comprises a segment of linked nucleosides, wherein each nucleoside of the segment comprises the same modified sugar moiety. An oligonucleotide having a uniformly modified sugar motif throughout the entirety of the oligonucleotide comprises only nucleosides comprising the same modified sugar moiety. For example, each nucleoside of a 2′-MOE uniformly modified oligonucleotide comprises a 2′-MOE modified sugar moiety. An oligonucleotide comprising or having a uniformly modified sugar motif can have any nucleobase sequence and any internucleoside linkage motif

B. Certain Nucleobase Motifs

In certain embodiments, oligomeric compounds described herein comprise or consist of oligonucleotides. In certain embodiments, oligonucleotides comprise modified and/or unmodified nucleobases arranged along the oligonucleotide or region thereof in a defined pattern or motif. In certain embodiments, each nucleobase is modified. In certain embodiments, none of the nucleobases are modified. In certain embodiments, each purine or each pyrimidine is modified. In certain embodiments, each adenine is modified. In certain embodiments, each guanine is modified. In certain embodiments, each thymine is modified. In certain embodiments, each uracil is modified. In certain embodiments, each cytosine is modified. In certain embodiments, some or all of the cytosine nucleobases in a modified oligonucleotide are 5-methylcytosines.

In certain embodiments, modified oligonucleotides comprise a block of modified nucleobases. In certain such embodiments, the block is at the 3′-end of the oligonucleotide. In certain embodiments the block is within 3 nucleosides of the 3′-end of the oligonucleotide. In certain embodiments, the block is at the 5′-end of the oligonucleotide. In certain embodiments the block is within 3 nucleosides of the 5′-end of the oligonucleotide.

C. Certain Internucleoside Linkage Motifs

In certain embodiments, oligomeric compounds described herein comprise or consist of oligonucleotides. In certain embodiments, oligonucleotides comprise modified and/or unmodified internucleoside linkages arranged along the oligonucleotide or region thereof in a defined pattern or motif. In certain embodiments, each internucleoside linkage is a phosphodiester internucleoside linkage (P═O). In certain embodiments, each internucleoside linkage of a modified oligonucleotide is a phosphorothioate internucleoside linkage (P═S). In certain embodiments, each internucleoside linkage of a modified oligonucleotide is independently selected from a phosphorothioate internucleoside linkage and phosphodiester internucleoside linkage. In certain embodiments, each phosphorothioate internucleoside linkage is independently selected from a stereorandom phosphorothioate, a (Sp) phosphorothioate, and a (Rp) phosphorothioate. In certain embodiments, the terminal internucleoside linkages are modified. In certain embodiments, the internucleoside linkage motif comprises at least one phosphodiester internucleoside linkage in at least one of the 5′-region and the 3′-region, wherein the at least one phosphodiester linkage is not a terminal internucleoside linkage, and the remaining internucleoside linkages are phosphorothioate internucleoside linkages. In certain such embodiments, all of the phosphorothioate linkages are stereorandom. In certain embodiments, populations of modified oligonucleotides are enriched for modified oligonucleotides comprising such internucleoside linkage motifs.

In certain embodiments, oligonucleotides comprise a region having an alternating internucleoside linkage motif. In certain embodiments, oligonucleotides comprise a region of uniformly modified internucleoside linkages. In certain such embodiments, the internucleoside linkages are phosphorothioate internucleoside linkages. In certain embodiments, all of the internucleoside linkages of the oligonucleotide are phosphorothioate internucleoside linkages. In certain embodiments, each internucleoside linkage of the oligonucleotide is selected from phosphodiester or phosphate and phosphorothioate. In certain embodiments, each internucleoside linkage of the oligonucleotide is selected from phosphodiester or phosphate and phosphorothioate and at least one internucleoside linkage is phosphorothioate.

In certain embodiments, the oligonucleotide comprises at least 6 phosphorothioate internucleoside linkages. In certain embodiments, the oligonucleotide comprises at least 8 phosphorothioate internucleoside linkages. In certain embodiments, the oligonucleotide comprises at least 10 phosphorothioate internucleoside linkages. In certain embodiments, the oligonucleotide comprises at least one block of at least 6 consecutive phosphorothioate internucleoside linkages. In certain embodiments, the oligonucleotide comprises at least one block of at least 8 consecutive phosphorothioate internucleoside linkages. In certain embodiments, the oligonucleotide comprises at least one block of at least 10 consecutive phosphorothioate internucleoside linkages. In certain embodiments, the oligonucleotide comprises at least block of at least one 12 consecutive phosphorothioate internucleoside linkages. In certain such embodiments, at least one such block is located at the 3′ end of the oligonucleotide. In certain such embodiments, at least one such block is located within 3 nucleosides of the 3′ end of the oligonucleotide.

In certain embodiments, oligonucleotides comprise one or more methylphosphonate linkages. In certain embodiments, modified oligonucleotides comprise a linkage motif comprising all phosphorothioate linkages except for one or two methylphosphonate linkages.

In certain embodiments, it is desirable to arrange the number of phosphorothioate internucleoside linkages and phosphodiester internucleoside linkages to maintain nuclease resistance. In certain embodiments, it is desirable to arrange the number and position of phosphorothioate internucleoside linkages and the number and position of phosphodiester internucleoside linkages to maintain nuclease resistance. In certain embodiments, the number of phosphorothioate internucleoside linkages may be decreased and the number of phosphodiester internucleoside linkages may be increased. In certain embodiments, the number of phosphorothioate internucleoside linkages may be decreased and the number of phosphodiester internucleoside linkages may be increased while still maintaining nuclease resistance. In certain embodiments it is desirable to decrease the number of phosphorothioate internucleoside linkages while retaining nuclease resistance. In certain embodiments it is desirable to increase the number of phosphodiester internucleoside linkages while retaining nuclease resistance.

III. Certain Modified Oligonucleotides

In certain embodiments, oligomeric compounds described herein comprise or consist of modified oligonucleotides. In certain embodiments, the above modifications (sugar, nucleobase, internucleoside linkage) are incorporated into a modified oligonucleotide. In certain embodiments, modified oligonucleotides are characterized by their modifications, motifs, and overall lengths. In certain embodiments, such parameters are each independent of one another. Thus, unless otherwise indicated, each internucleoside linkage of a modified oligonucleotide may be modified or unmodified and may or may not follow the modification pattern of the sugar moieties. Likewise, such modified oligonucleotides may comprise one or more modified nucleobase independent of the pattern of the sugar modifications. Furthermore, in certain instances, a modified oligonucleotide is described by an overall length or range and by lengths or length ranges of two or more regions (e.g., a region of nucleosides having specified sugar modifications), in such circumstances it may be possible to select numbers for each range that result in an oligonucleotide having an overall length falling outside the specified range. In such circumstances, both elements must be satisfied. For example, in certain embodiments, a modified oligonucleotide consists of 15-20 linked nucleosides and has a sugar motif consisting of three regions or segments, A, B, and C, wherein region or segment A consists of 2-6 linked nucleosides having a specified sugar motif, region or segment B consists of 6-10 linked nucleosides having a specified sugar motif, and region or segment C consists of 2-6 linked nucleosides having a specified sugar motif. Such embodiments do not include modified oligonucleotides where A and C each consist of 6 linked nucleosides and B consists of 10 linked nucleosides (even though those numbers of nucleosides are permitted within the requirements for A, B, and C) because the overall length of such oligonucleotide is 22, which exceeds the upper limit of 20 for the overall length of the modified oligonucleotide. Unless otherwise indicated, all modifications are independent of nucleobase sequence except that the modified nucleobase 5-methylcytosine is necessarily a “C” in an oligonucleotide sequence.

In certain embodiments, oligonucleotides consist of X to Y linked nucleosides, where X represents the fewest number of nucleosides in the range and Y represents the largest number nucleosides in the range. In certain such embodiments, X and Y are each independently selected from 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50; provided that X≤Y. For example, in certain embodiments, oligonucleotides consist of 12 to 13, 12 to 14, 12 to 15, 12 to 16, 12 to 17, 12 to 18, 12 to 19, 12 to 20, 12 to 21, 12 to 22, 12 to 23, 12 to 24, 12 to 25, 12 to 26, 12 to 27, 12 to 28, 12 to 29, 12 to 30, 13 to 14, 13 to 15, 13 to 16, 13 to 17, 13 to 18, 13 to 19, 13 to 20, 13 to 21, 13 to 22, 13 to 23, 13 to 24, 13 to 25, 13 to 26, 13 to 27, 13 to 28, 13 to 29, 13 to 30, 14 to 15, 14 to 16, 14 to 17, 14 to 18, 14 to 19, 14 to 20, 14 to 21, 14 to 22, 14 to 23, 14 to 24, 14 to 25, 14 to 26, 14 to 27, 14 to 28, 14 to 29, 14 to 30, 15 to 16, 15 to 17, 15 to 18, 15 to 19, 15 to 20, 15 to 21, 15 to 22, 15 to 23, 15 to 24, 15 to 25, 15 to 26, 15 to 27, 15 to 28, 15 to 29, 15 to 30, 16 to 17, 16 to 18, 16 to 19, 16 to 20, 16 to 21, 16 to 22, 16 to 23, 16 to 24, 16 to 25, 16 to 26, 16 to 27, 16 to 28, 16 to 29, 16 to 30, 17 to 18, 17 to 19, 17 to 20, 17 to 21, 17 to 22, 17 to 23, 17 to 24, 17 to 25, 17 to 26, 17 to 27, 17 to 28, 17 to 29, 17 to 30, 18 to 19, 18 to 20, 18 to 21, 18 to 22, 18 to 23, 18 to 24, 18 to 25, 18 to 26, 18 to 27, 18 to 28, 18 to 29, 18 to 30, 19 to 20, 19 to 21, 19 to 22, 19 to 23, 19 to 24, 19 to 25, 19 to 26, 19 to 29, 19 to 28, 19 to 29, 19 to 30, 20 to 21, 20 to 22, 20 to 23, 20 to 24, 20 to 25, 20 to 26, 20 to 27, 20 to 28, 20 to 29, 20 to 30, 21 to 22, 21 to 23, 21 to 24, 21 to 25, 21 to 26, 21 to 27, 21 to 28, 21 to 29, 21 to 30, 22 to 23, 22 to 24, 22 to 25, 22 to 26, 22 to 27, 22 to 28, 22 to 29, 22 to 30, 23 to 24, 23 to 25, 23 to 26, 23 to 27, 23 to 28, 23 to 29, 23 to 30, 24 to 25, 24 to 26, 24 to 27, 24 to 28, 24 to 29, 24 to 30, 25 to 26, 25 to 27, 25 to 28, 25 to 29, 25 to 30, 26 to 27, 26 to 28, 26 to 29, 26 to 30, 27 to 28, 27 to 29, 27 to 30, 28 to 29, 28 to 30, or 29 to 30 linked nucleosides.

In certain embodiments oligonucleotides have a nucleobase sequence that is complementary to a second oligonucleotide or an identified reference nucleic acid, such as a target nucleic acid. In certain embodiments, a region of an oligonucleotide has a nucleobase sequence that is complementary to a second oligonucleotide or an identified reference nucleic acid, such as a target nucleic acid. In certain embodiments, the nucleobase sequence of a region or entire length of an oligonucleotide is at least 70%, at least 80%, at least 90%, at least 95%, or 100% complementary to the second oligonucleotide or nucleic acid, such as a target nucleic acid.

IV. Certain Conjugated Compounds

In certain embodiments, the oligomeric compounds described herein comprise or consist of an oligonucleotide (modified or unmodified) and optionally one or more conjugate groups and/or terminal groups. Conjugate groups consist of one or more conjugate moiety and a conjugate linker that links the conjugate moiety to the oligonucleotide. Conjugate groups may be attached to either or both ends of an oligonucleotide and/or at any internal position. In certain embodiments, conjugate groups are attached to the 2′-position of a nucleoside of a modified oligonucleotide. In certain embodiments, conjugate groups that are attached to either or both ends of an oligonucleotide are terminal groups. In certain such embodiments, conjugate groups or terminal groups are attached at the 3′ and/or 5′-end of oligonucleotides. In certain such embodiments, conjugate groups (or terminal groups) are attached at the 3′-end of oligonucleotides. In certain embodiments, conjugate groups are attached near the 3′-end of oligonucleotides. In certain embodiments, conjugate groups (or terminal groups) are attached at the 5′-end of oligonucleotides. In certain embodiments, conjugate groups are attached near the 5′-end of oligonucleotides.

Examples of terminal groups include but are not limited to conjugate groups, capping groups, phosphate moieties, protecting groups, modified or unmodified nucleosides, and two or more nucleosides that are independently modified or unmodified.

A. Certain Conjugate Groups

In certain embodiments, oligonucleotides are covalently attached to one or more conjugate groups. In certain embodiments, conjugate groups modify one or more properties of the attached oligonucleotide, including but not limited to pharmacodynamics, pharmacokinetics, stability, binding, absorption, tissue distribution, cellular distribution, cellular uptake, charge and clearance. In certain embodiments, conjugate groups impart a new property on the attached oligonucleotide, e.g., fluorophores or reporter groups that enable detection of the oligonucleotide.

Certain conjugate groups and conjugate moieties have been described previously, for example: cholesterol moiety (Letsinger et al., Proc. Natl. Acad. Sci. USA, 1989, 86, 6553-6556), cholic acid (Manoharan et al., Bioorg. Med. Chem. Lett., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Lett., 1993, 3, 2765-2770), a thiocholesterol (Oberhauser et al., Nucl. Acids Res., 1992, 20, 533-538), an aliphatic chain, e.g., do-decan-diol or undecyl residues (Saison-Behmoaras et al., EMBO 1, 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic, a palmityl moiety (Mishra et al., Biochim. Biophys. Acta, 1995, 1264, 229-237), an octadecylamine or hexylamino-carbonyl-oxycholesterol moiety (Crooke et al., J. Pharmacol. Exp. Ther., 1996, i, 923-937),₌a tocopherol group (Nishina et al., Molecular Therapy Nucleic Acids, 2015, 4, e220; doi:10.1038/mtna.2014.72 and Nishina et al., Molecular Therapy, 2008, 16, 734-740), or a GalNAc cluster (e.g., WO2014/179620).

1. Conjugate Moieties

Conjugate moieties include, without limitation, intercalators, reporter molecules, polyamines, polyamides, peptides, carbohydrates (e.g., GalNAc), vitamin moieties, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins, fluorophores, and dyes.

In certain embodiments, a conjugate moiety comprises an active drug substance, for example, aspirin, warfarin, phenylbutazone, ibuprofen, suprofen, fen-bufen, ketoprofen, (S)-(+)-pranoprofen, carprofen, dansylsarcosine, 2,3,5-triiodobenzoic acid, fingolimod, flufenamic acid, folinic acid, a benzothiadiazide, chlorothiazide, a diazepine, indo-methicin, a barbiturate, a cephalosporin, a sulfa drug, an antidiabetic, an antibacterial or an antibiotic.

2. Conjugate Linkers

Conjugate moieties are attached to oligonucleotides through conjugate linkers. In certain oligomeric compounds, a conjugate linker is a single chemical bond (i.e. conjugate moiety is attached to an oligonucleotide via a conjugate linker through a single bond). In certain embodiments, the conjugate linker comprises a chain structure, such as a hydrocarbyl chain, or an oligomer of repeating units such as ethylene glycol, nucleosides, or amino acid units.

In certain embodiments, a conjugate linker comprises one or more groups selected from alkyl, amino, oxo, amide, disulfide, polyethylene glycol, ether, thioether, and hydroxylamino. In certain such embodiments, the conjugate linker comprises groups selected from alkyl, amino, oxo, amide and ether groups. In certain embodiments, the conjugate linker comprises groups selected from alkyl and amide groups. In certain embodiments, the conjugate linker comprises groups selected from alkyl and ether groups. In certain embodiments, the conjugate linker comprises at least one phosphorus moiety. In certain embodiments, the conjugate linker comprises at least one phosphate group. In certain embodiments, the conjugate linker includes at least one neutral linking group.

In certain embodiments, conjugate linkers, including the conjugate linkers described above, are bifunctional linking moieties, e.g., those known in the art to be useful for attaching conjugate groups to oligomeric compounds, such as the oligonucleotides provided herein. In general, a bifunctional linking moiety comprises at least two functional groups. One of the functional groups is selected to bind to a particular site on an oligomeric compound and the other is selected to bind to a conjugate group. Examples of functional groups used in a bifunctional linking moiety include but are not limited to electrophiles for reacting with nucleophilic groups and nucleophiles for reacting with electrophilic groups. In certain embodiments, bifunctional linking moieties comprise one or more groups selected from amino, hydroxyl, carboxylic acid, thiol, alkyl, alkenyl, and alkynyl.

Examples of conjugate linkers include but are not limited to pyrrolidine, 8-amino-3,6-dioxaoctanoic acid (ADO), succinimidyl 4-(N-maleimidomethyl) cyclohexane-l-carboxylate (SMCC) and 6-aminohexanoic acid (AHEX or AHA). Other conjugate linkers include but are not limited to substituted or unsubstituted C₁-C₁₀ alkyl, substituted or unsubstituted C₂-C₁₀ alkenyl or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

In certain embodiments, conjugate linkers comprise 1-10 linker-nucleosides. In certain embodiments, such linker-nucleosides are modified nucleosides. In certain embodiments such linker-nucleosides comprise a modified sugar moiety. In certain embodiments, linker-nucleosides are unmodified. In certain embodiments, linker-nucleosides comprise an optionally protected heterocyclic base selected from a purine, substituted purine, pyrimidine or substituted pyrimidine. In certain embodiments, a cleavable moiety is a nucleoside selected from uracil, thymine, cytosine, 4-N-benzoylcytosine, 5-methylcytosine, 4-N-benzoyl-5-methylcytosine, adenine, 6-N-benzoyladenine, guanine and 2-N-isobutyrylguanine. It is typically desirable for linker-nucleosides to be cleaved from the oligomeric compound after it reaches a target tissue. Accordingly, linker-nucleosides are typically linked to one another and to the remainder of the oligomeric compound through cleavable bonds. In certain embodiments, such cleavable bonds are phosphodiester bonds.

Herein, linker-nucleosides are not considered to be part of the oligonucleotide. Accordingly, in embodiments in which an oligomeric compound comprises an oligonucleotide consisting of a specified number or range of linked nucleosides and/or a specified percent complementarity to a reference nucleic acid and the oligomeric compound also comprises a conjugate group comprising a conjugate linker comprising linker-nucleosides, those linker-nucleosides are not counted toward the length of the oligonucleotide and are not used in determining the percent complementarity of the oligonucleotide for the reference nucleic acid. For example, an oligomeric compound may comprise (1) a modified oligonucleotide consisting of 8-30 nucleosides and (2) a conjugate group comprising 1-10 linker-nucleosides that are contiguous with the nucleosides of the modified oligonucleotide. The total number of contiguous linked nucleosides in such a compound is more than 30. Alternatively, an oligomeric compound may comprise a modified oligonucleotide consisting of 8-30 nucleosides and no conjugate group. The total number of contiguous linked nucleosides in such a compound is no more than 30. Unless otherwise indicated conjugate linkers comprise no more than 10 linker-nucleosides. In certain embodiments, conjugate linkers comprise no more than 5 linker-nucleosides. In certain embodiments, conjugate linkers comprise no more than 3 linker-nucleosides. In certain embodiments, conjugate linkers comprise no more than 2 linker-nucleosides. In certain embodiments, conjugate linkers comprise no more than 1 linker-nucleoside.

In certain embodiments, it is desirable for a conjugate group to be cleaved from the oligonucleotide. For example, in certain circumstances oligomeric compounds comprising a particular conjugate moiety are better taken up by a particular cell type, but once the compound has been taken up, it is desirable that the conjugate group be cleaved to release the unconjugated oligonucleotide. Thus, certain conjugate may comprise one or more cleavable moieties, typically within the conjugate linker. In certain embodiments, a cleavable moiety is a cleavable bond. In certain embodiments, a cleavable moiety is a group of atoms comprising at least one cleavable bond. In certain embodiments, a cleavable moiety comprises a group of atoms having one, two, three, four, or more than four cleavable bonds. In certain embodiments, a cleavable moiety is selectively cleaved inside a cell or subcellular compartment, such as a lysosome. In certain embodiments, a cleavable moiety is selectively cleaved by endogenous enzymes, such as nucleases.

In certain embodiments, a cleavable bond is selected from among: an amide, an ester, an ether, one or both esters of a phosphodiester, a phosphate ester, a carbamate, or a disulfide. In certain embodiments, a cleavable bond is one or both of the esters of a phosphodiester. In certain embodiments, a cleavable moiety comprises a phosphate or phosphodiester. In certain embodiments, the cleavable moiety is a phosphate linkage between an oligonucleotide and a conjugate moiety or conjugate group.

In certain embodiments, a cleavable moiety comprises or consists of one or more linker-nucleosides. In certain such embodiments, one or more linker-nucleosides are linked to one another and/or to the remainder of the oligomeric compound through cleavable bonds. In certain embodiments, such cleavable bonds are unmodified phosphodiester bonds. In certain embodiments, a cleavable moiety is 2′-deoxy nucleoside that is attached to either the 3′ or 5′-terminal nucleoside of an oligonucleotide by a phosphate internucleoside linkage and covalently attached to the remainder of the conjugate linker or conjugate moiety by a phosphate or phosphorothioate linkage. In certain such embodiments, the cleavable moiety is 2′-deoxyadenosine.

3. Certain Cell-Targeting Conjugate Moieties

In certain embodiments, a conjugate group comprises a cell-targeting conjugate moiety. In certain embodiments, a conjugate group has the general formula:

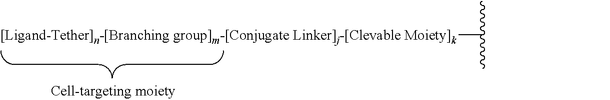

wherein n is from 1 to about 3, m is 0 when n is 1, m is 1 when n is 2 or greater, j is 1 or 0, and k is 1 or 0.

In certain embodiments, n is 1, j is 1 and k is 0. In certain embodiments, n is 1, j is 0 and k is 1. In certain embodiments, n is 1, j is 1 and k is 1. In certain embodiments, n is 2, j is 1 and k is 0. In certain embodiments, n is 2, j is 0 and k is 1. In certain embodiments, n is 2, j is 1 and k is 1. In certain embodiments, n is 3, j is 1 and k is 0. In certain embodiments, n is 3, j is 0 and k is 1. In certain embodiments, n is 3, j is 1 and k is 1.

In certain embodiments, conjugate groups comprise cell-targeting moieties that have at least one tethered ligand. In certain embodiments, cell-targeting moieties comprise two tethered ligands covalently attached to a branching group. In certain embodiments, cell-targeting moieties comprise three tethered ligands covalently attached to a branching group.

In certain embodiments, the cell-targeting moiety comprises a branching group comprising one or more groups selected from alkyl, amino, oxo, amide, disulfide, polyethylene glycol, ether, thioether and hydroxylamino groups. In certain embodiments, the branching group comprises a branched aliphatic group comprising groups selected from alkyl, amino, oxo, amide, disulfide, polyethylene glycol, ether, thioether and hydroxylamino groups. In certain such embodiments, the branched aliphatic group comprises groups selected from alkyl, amino, oxo, amide and ether groups. In certain such embodiments, the branched aliphatic group comprises groups selected from alkyl, amino and ether groups. In certain such embodiments, the branched aliphatic group comprises groups selected from alkyl and ether groups. In certain embodiments, the branching group comprises a mono or polycyclic ring system.

In certain embodiments, each tether of a cell-targeting moiety comprises one or more groups selected from alkyl, substituted alkyl, ether, thioether, disulfide, amino, oxo, amide, phosphodiester, and polyethylene glycol, in any combination. In certain embodiments, each tether is a linear aliphatic group comprising one or more groups selected from alkyl, ether, thioether, disulfide, amino, oxo, amide, and polyethylene glycol, in any combination. In certain embodiments, each tether is a linear aliphatic group comprising one or more groups selected from alkyl, phosphodiester, ether, amino, oxo, and amide, in any combination. In certain embodiments, each tether is a linear aliphatic group comprising one or more groups selected from alkyl, ether, amino, oxo, and amid, in any combination. In certain embodiments, each tether is a linear aliphatic group comprising one or more groups selected from alkyl, amino, and oxo, in any combination. In certain embodiments, each tether is a linear aliphatic group comprising one or more groups selected from alkyl and oxo, in any combination. In certain embodiments, each tether is a linear aliphatic group comprising one or more groups selected from alkyl and phosphodiester, in any combination. In certain embodiments, each tether comprises at least one phosphorus linking group or neutral linking group. In certain embodiments, each tether comprises a chain from about 6 to about 20 atoms in length. In certain embodiments, each tether comprises a chain from about 10 to about 18 atoms in length. In certain embodiments, each tether comprises about 10 atoms in chain length.

In certain embodiments, each ligand of a cell-targeting moiety has an affinity for at least one type of receptor on a target cell. In certain embodiments, each ligand has an affinity for at least one type of receptor on the surface of a mammalian lung cell.

In certain embodiments, each ligand of a cell-targeting moiety is a carbohydrate, carbohydrate derivative, modified carbohydrate, polysaccharide, modified polysaccharide, or polysaccharide derivative. In certain such embodiments, the conjugate group comprises a carbohydrate cluster (see, e.g., Maier et al., “Synthesis of Antisense Oligonucleotides Conjugated to a Multivalent Carbohydrate Cluster for Cellular Targeting,” Bioconjugate Chemistry, 2003, 14, 18-29, or Rensen et al., “Design and Synthesis of Novel N-Acetylgalactosamine-Terminated Glycolipids for Targeting of Lipoproteins to the Hepatic Asiaglycoprotein Receptor,” J. Med. Chem. 2004, 47, 5798-5808, which are incorporated herein by reference in their entirety). In certain such embodiments, each ligand is an amino sugar or a thio sugar. For example, amino sugars may be selected from any number of compounds known in the art, such as sialic acid, α-D-galactosamine, β-muramic acid, 2-deoxy-2-methylamino-L-glucopyranose, 4,6-dideoxy-4-formamido-2,3-di-O-methyl-D-mannopyranose, 2-deoxy-2-sulfoamino-D-glucopyranose and N-sulfo-D-glucosamine, and N-glycoloyl-α-neuraminic acid. For example, thio sugars may be selected from 5-Thio-β-D-glucopyranose, methyl 2,3,4-tri-O-acetyl-1-thio-6-O-trityl-α-D-glucopyranoside, 4-thio-β-D-galactopyranose, and ethyl 3,4,6,7-tetra-O-acetyl-2-deoxy-1,5-dithio-α-D-gluco-heptopyranoside.

In certain embodiments, oligomeric compounds described herein comprise a conjugate group found in any of the following references: Lee, Carbohydr Res, 1978, 67, 509-514; Connolly et al., J Biol Chem, 1982, 257, 939-945; Pavia et al., Int J Pep Protein Res, 1983, 22, 539-548; Lee et al., Biochem, 1984, 23, 4255-4261; Lee et al., Glycoconjugate J, 1987, 4, 317-328; Toyokuni et al., Tetrahedron Lett, 1990, 31, 2673-2676; Biessen et al., J Med Chem, 1995, 38, 1538-1546; Valentijn et al., Tetrahedron, 1997, 53, 759-770; Kim et al., Tetrahedron Lett, 1997, 38, 3487-3490; Lee et al., Bioconjug Chem, 1997, 8, 762-765; Kato et al., Glycobiol, 2001, 11, 821-829; Rensen et al., J Biol Chem, 2001, 276, 37577-37584; Lee et al., Methods Enzymol, 2003, 362, 38-43; Westerlind et al., Glycoconj J, 2004, 21, 227-241; Lee et al., Bioorg Med Chem Lett, 2006, 16(19), 5132-5135; Maierhofer et al., Bioorg Med Chem, 2007, 15, 7661-7676; Khorev et al., Bioorg Med Chem, 2008, 16, 5216-5231; Lee et al., Bioorg Med Chem, 2011, 19, 2494-2500; Kornilova et al., Analyt Biochem, 2012, 425, 43-46; Pujol et al., Angew Chemie Int Ed Engl, 2012, 51, 7445-7448; Biessen et al., J Med Chem, 1995, 38, 1846-1852; Sliedregt et al., J Med Chem, 1999, 42, 609-618; Rensen et al., J Med Chem, 2004, 47, 5798-5808; Rensen et al., Arterioscler Thromb Vasc Biol, 2006, 26, 169-175; van Rossenberg et al., Gene Ther, 2004, 11, 457-464; Sato et al., J Am Chem Soc, 2004, 126, 14013-14022; Lee et al., J Org Chem, 2012, 77, 7564-7571; Biessen et al., FASEB J, 2000, 14, 1784-1792; Rajur et al., Bioconjug Chem, 1997, 8, 935-940; Duff et al., Methods Enzymol, 2000, 313, 297-321; Maier et al., Bioconjug Chem, 2003, 14, 18-29; Jayaprakash et al., Org Lett, 2010, 12, 5410-5413; Manoharan, Antisense Nucleic Acid Drug Dev, 2002, 12, 103-128; Merwin et al., Bioconjug Chem, 1994, 5, 612-620; Tomiya et al., Bioorg Med Chem, 2013, 21, 5275-5281; International applications WO1998/013381; WO2011/038356; WO1997/046098; WO2008/098788; WO2004/101619; WO2012/037254; WO2011/120053; WO2011/100131; WO2011/163121; WO2012/177947; WO2013/033230; WO2013/075035; WO2012/083185; WO2012/083046; WO2009/082607; WO2009/134487; WO2010/144740; WO2010/148013; WO1997/020563; WO2010/088537; WO2002/043771; WO2010/129709; WO2012/068187; WO2009/126933; WO2004/024757; WO2010/054406; WO2012/089352; WO2012/089602; WO2013/166121; WO2013/165816; U.S. Pat. Nos. 4,751,219; 8,552,163; 6,908,903; 7,262,177; 5,994,517; 6,300,319; 8,106,022; 7,491,805; 7,491,805; 7,582,744; 8,137,695; 6,383,812; 6,525,031; 6,660,720; 7,723,509; 8,541,548; 8,344,125; 8,313,772; 8,349,308; 8,450,467; 8,501,930; 8,158,601; 7,262,177; 6,906,182; 6,620,916; 8,435,491; 8,404,862; 7,851,615; Published U.S. Patent Application Publications US2011/0097264; US2011/0097265; US2013/0004427; US2005/0164235; US2006/0148740; US2008/0281044; US2010/0240730; US2003/0119724; US2006/0183886; US2008/0206869; US2011/0269814; US2009/0286973; US2011/0207799; US2012/0136042; US2012/0165393; US2008/0281041; US2009/0203135; US2012/0035115; US2012/0095075; US2012/0101148; US2012/0128760; US2012/0157509; US2012/0230938; US2013/0109817; US2013/0121954; US2013/0178512; US2013/0236968; US2011/0123520; US2003/0077829; US2008/0108801; and US2009/0203132.

### Compositions and Methods for Formulating Pharmaceutical Compositions

Oligomeric compounds described herein may be admixed with pharmaceutically acceptable active or inert substances for the preparation of pharmaceutical compositions. Compositions and methods for the formulation of pharmaceutical compositions are dependent upon a number of criteria, including, but not limited to, route of administration, extent of disease, or dose to be administered.

Certain embodiments provide pharmaceutical compositions comprising one or more oligomeric compounds or a salt thereof. In certain embodiments, the oligomeric compounds comprise or consist of a modified oligonucleotide. In certain such embodiments, the pharmaceutical composition comprises a suitable pharmaceutically acceptable diluent or carrier. In certain embodiments, a pharmaceutical composition comprises a sterile saline solution and one or more oligomeric compound. In certain embodiments, such pharmaceutical composition consists of a sterile saline solution and one or more oligomeric compound. In certain embodiments, the sterile saline is pharmaceutical grade saline. In certain embodiments, a pharmaceutical composition comprises one or more oligomeric compound and sterile water. In certain embodiments, a pharmaceutical composition consists of one oligomeric compound and sterile water. In certain embodiments, the sterile water is pharmaceutical grade water. In certain embodiments, a pharmaceutical composition comprises or consists of one or more oligomeric compound and phosphate-buffered saline (PBS). In certain embodiments, a pharmaceutical composition consists of one or more oligomeric compound and sterile PBS. In certain embodiments, the sterile PBS is pharmaceutical grade PBS. Compositions and methods for the formulation of pharmaceutical compositions are dependent upon a number of criteria, including, but not limited to, route of administration, extent of disease, or dose to be administered.

An oligomeric compound described herein complementary to a target nucleic acid can be utilized in pharmaceutical compositions by combining the oligomeric compound with a suitable pharmaceutically acceptable diluent or carrier and/or additional components such that the pharmaceutical composition is suitable for injection. In certain embodiments, a pharmaceutically acceptable diluent is phosphate buffered saline. Accordingly, in one embodiment, employed in the methods described herein is a pharmaceutical composition comprising an oligomeric compound complementary to a target nucleic acid and a pharmaceutically acceptable diluent. In certain embodiments, the pharmaceutically acceptable diluent is phosphate buffered saline. In certain embodiments, the oligomeric compound comprises or consists of a modified oligonucleotide provided herein.

Pharmaceutical compositions comprising oligomeric compounds provided herein encompass any pharmaceutically acceptable salts, esters, or salts of such esters, or any other oligonucleotide which, upon administration to an animal, including a human, is capable of providing (directly or indirectly) the biologically active metabolite or residue thereof. In certain embodiments, the oligomeric compound comprises or consists of a modified oligonucleotide. Accordingly, for example, the disclosure is also drawn to pharmaceutically acceptable salts of compounds, prodrugs, pharmaceutically acceptable salts of such prodrugs, and other bioequivalents. Suitable pharmaceutically acceptable salts include, but are not limited to, sodium and potassium salts.

### Certain Mechanisms

Site specific introduction of (Sp) or (Rp) phosphorothioate internucleoside linkages into the central region of a modified oligonucleotide can alter the protein binding properties of the modified oligonucleotide.

In certain embodiments, site specific introduction of (Sp) or (Rp) phosphorothioate internucleoside linkages into the central region of a modified oligonucleotide can alter the cleavage pattern of RNase H1 when the modified oligonucleotide binds to a target nucleic acid. In certain embodiments, site specific introduction of (Sp) or (Rp) phosphorothioate internucleoside linkages into the central region of a modified oligonucleotide can increase the therapeutic index of a modified oligonucleotide having a stereorandom configuration at the phosphorothioate internucleoside linkages.

In certain embodiments, site specific introduction of (Sp) or (Rp) phosphorothioate internucleoside linkages into the central region of a modified oligonucleotide can alter the protein binding properties of the modified oligonucleotide. This can also serve to increase the activity of the modified oligonucleotide and/or improve the therapeutic index of a modified oligonucleotide. Site specific introduction of (Sp) or (Rp) phosphorothioate internucleoside linkages into the central region of a modified oligonucleotide can weaken the interaction of the modified oligonucletodie with certain proteins associated with decreased antisense activity. Site specific introduction of (Sp) or (Rp) phosphorothioate internucleoside linkages into the central region of a modified oligonucleotide can increase or strengthen the interaction of the modified oligonucleotide with certain proteins associated with increased antisense activity.

In certain embodiments, specific patterns of (Sp) or (Rp) phosphorothioate internucleoside linkages into the central region of a modified oligonucleotide can increase the therapeutic index compared to a modified oligonucleotide having stereorandom internucleoside linkages. For example, in certain embodiments, placement of a single (Rp) phosphorothioate internucleoside linkage into the 3′-end of the central region of a modified oligonucleotide can increase the activity of a modified oligonucleotide. In certain embodiments, having a single (Rp) phosphorothioate internucleoside linkage in a central region wherein each remaining phosphorothioate internucleoside linkage has the (Sp) configuration improves the therapeutic index of the modified oligonucleotide.

### Target Nucleic Acids

In certain embodiments, compounds described herein comprise or consist of an oligonucleotide that is complementary to a target nucleic acid. In certain embodiments, the target nucleic acid is an endogenous RNA molecule. In certain embodiments, the target nucleic acid encodes a protein. In certain such embodiments, the target nucleic acid is an mRNA. In certain embodiments, an oligonucleotide is complementary to both a pre-mRNA and corresponding mRNA but only the mRNA is the target nucleic acid due to an absence of antisense activity upon hybridization to the pre-mRNA. In certain embodiments, an oligonucleotide is complementary to an exon-exon junction of a target mRNA and is not complementary to the corresponding pre-mRNA.

### Compound Isomers

Certain compounds described herein (e.g., modified oligonucleotides) have one or more asymmetric center and thus give rise to enantiomers, diastereomers, and other stereoisomeric configurations that may be defined, in terms of absolute stereochemistry, as (R) or (S), as α or β, such as for sugar anomers, or as (D) or (L), such as for amino acids, etc. Compounds provided herein that are drawn or described as having certain stereoisomeric configurations include only the indicated compounds. Compounds provided herein that are drawn or described with undefined stereochemistry include all such possible isomers, including their stereorandom and optically pure forms. All tautomeric forms of the compounds provided herein are included unless otherwise indicated.

The compounds described herein include variations in which one or more atoms are replaced with a non-radioactive isotope or radioactive isotope of the indicated element. For example, compounds herein that comprise hydrogen atoms encompass all possible deuterium substitutions for each of the ¹H hydrogen atoms. Isotopic substitutions encompassed by the compounds herein include but are not limited to: ²H or ³H in place of ¹H, ¹³C or ¹⁴C in place of ¹⁵N in place of ¹⁴N, ¹⁷O or ¹⁸O in place of ¹⁶O, and ³³S, ³⁴S, ³⁵S, or ³⁶S in place of ³²S. In certain embodiments, non-radioactive isotopic substitutions may impart new properties on the oligomeric compound that are beneficial for use as a therapeutic or research tool. In certain embodiments, radioactive isotopic substitutions may make the compound suitable for research or diagnostic purposes such as imaging.

### EXAMPLES

### Non-limiting Disclosure and Incorporation by Reference

Although the sequence listing accompanying this filing identifies each sequence as either “RNA” or “DNA” as required, in reality, those sequences may be modified with any combination of chemical modifications. One of skill in the art will readily appreciate that such designation as “RNA” or “DNA” to describe modified oligonucleotides is, in certain instances, arbitrary. For example, an oligonucleotide comprising a nucleoside comprising a 2′-OH sugar moiety and a thymine nucleobase could be described as a DNA having an RNA sugar, or as an RNA having a DNA nucleobase.

Accordingly, nucleic acid sequences provided herein, including, but not limited to those in the sequence listing, are intended to encompass nucleic acids containing any combination of unmodified or modified RNA and/or DNA, including, but not limited to such nucleic acids having modified nucleobases. By way of further example and without limitation, an oligonucleotide having the nucleobase sequence “ATCGATCG” encompasses any oligonucleotides having such nucleobase sequence, whether modified or unmodified, including, but not limited to, such compounds comprising RNA bases, such as those having sequence “AUCGAUCG” and those having some DNA bases and some RNA bases such as “AUCGATCG” and compounds having other modified nucleobases, such as “AT^{m}CGAUCG,” wherein ^{m}C indicates a cytosine base comprising a methyl group at the 5-position.

While certain compounds, compositions and methods described herein have been described with specificity in accordance with certain embodiments, the following examples serve only to illustrate the compounds described herein and are not intended to limit the same. Each of the references recited in the present application is incorporated herein by reference in its entirety.

### Example 1 Nucleosides with Chiral Phosphorothioate Linkages

Modified oligonucleotides containing chirally-controlled phosphorothioate linkages in the central region were synthesized. The compounds in the table below are 100% complementary to mouse CXCL12, GENBANK NT_039353.7 truncated from 69430515 to 69445350 (SEQ ID NO: 1), at position 6877 to 6892. Each compound has the kkk-d(10)-kkk sugar motif, wherein each “k” represents a 2′-constrained ethyl modified sugar moiety and each “d” represents a 2′-deoxy sugar moiety. Internucleoside linkages 1, 2, 3, 14, and 15 are stereorandom phosphorothioate linkages. Internucleoside linkages 4-13 have the stereochemistry indicated in the table below, wherein a subscript “s” indicates a stereorandom phosphorothioate internucleoside linkage, a subscript “r” indicates a phosphorothioate internucleoside linkage having the (Rp) configuration and a subscript “q” indicates a phosphorothioate internucleoside linkage having the (Sp) configuration.

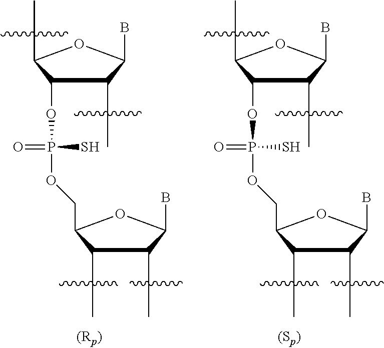

TABLE 1
Modified oligonucleotides with stereochemically-controlled
phosphorothioate linkages

| Compound | | SEQ |
|---|---|---|
| ID | Chemistry Notation | ID NO |
| 558807 | Gₖₛ^{m}CₖₛAₖₛT_{ds}G_{ds}T_{ds}T_{ds}^{m}C_{ds}T_{ds}^{m}C_{ds}A_{ds}^{m}C_{ds}A_{ds}TₖₛTₖₛAₖ | 5 |
| 1220041 | Gₖₛ^{m}CₖₛAₖₛT_{dq}G_{dq}T_{dq}T_{dq}^{m}C_{dq}T_{dq}^{m}C_{dq}A_{dq}^{m}C_{dq}A_{dq}TₖₛTₖₛAₖ | 5 |
| 1220042 | Gₖₛ^{m}CₖₛAₖₛT_{dr}G_{dq}T_{dq}T_{dq}^{m}C_{dq}T_{dq}^{m}C_{dq}A_{dq}^{m}C_{dq}A_{dq}TₖₛTₖₛAₖ | 5 |
| 1220043 | Gₖₛ^{m}CₖₛAₖₛT_{dq}G_{dr}T_{dq}T_{dq}^{m}C_{dq}T_{dq}^{m}C_{dq}A_{dq}^{m}C_{dq}A_{dq}TₖₛTₖₛAₖ | 5 |
| 1220044 | Gₖₛ^{m}CₖₛAₖₛT_{dq}G_{dq}T_{dr}T_{dq}^{m}C_{dq}T_{dq}^{m}C_{dq}A_{dq}^{m}C_{dq}A_{dq}TₖₛTₖₛAₖ | 5 |
| 1220045 | Gₖₛ^{m}CₖₛAₖₛT_{dq}G_{dq}T_{dq}T_{dr}^{m}C_{dq}T_{dq}^{m}C_{dq}A_{dq}^{m}C_{dq}A_{dq}TₖₛTₖₛAₖ | 5 |
| 1220046 | Gₖₛ^{m}CₖₛAₖₛT_{dq}G_{dq}T_{dq}T_{dq}^{m}C_{dr}T_{dq}^{m}C_{dq}A_{dq}^{m}C_{dq}A_{dq}TₖₛTₖₛAₖ | 5 |
| 1220051 | Gₖₛ^{m}CₖₛAₖₛT_{dq}G_{dq}T_{dq}T_{dq}^{m}C_{dq}T_{dr}^{m}C_{dq}A_{dq}^{m}C_{dq}A_{dq}TₖₛTₖₛAₖ | 5 |
| 1220047 | Gₖₛ^{m}CₖₛAₖₛT_{dq}G_{dq}T_{dq}T_{dq}^{m}C_{dq}T_{dq}^{m}C_{dr}A_{dq}^{m}C_{dq}A_{dq}TₖₛTₖₛAₖ | 5 |
| 1220048 | Gₖₛ^{m}CₖₛAₖₛT_{dq}G_{dq}T_{dq}T_{dq}^{m}C_{dq}T_{dq}^{m}C_{dq}A_{dr}^{m}C_{dq}A_{dq}TₖₛTₖₛAₖ | 5 |
| 1220049 | Gₖₛ^{m}CₖₛAₖₛT_{dq}G_{dq}T_{dq}T_{dq}^{m}C_{dq}T_{dq}^{m}C_{dq}A_{dq}^{m}C_{dr}A_{dq}TₖₛTₖₛAₖ | 5 |
| 1220050 | Gₖₛ^{m}CₖₛAₖₛT_{dq}G_{dq}T_{dq}T_{dq}^{m}C_{dq}T_{dq}^{m}C_{dq}A_{dq}^{m}C_{dq}A_{dr}TₖₛTₖₛAₖ | 5 |
| 1237987 | Gₖₛ^{m}CₖₛAₖₛT_{dq}G_{dq}T_{dq}T_{dq}^{m}C_{dq}T_{dq}^{m}C_{dq}A_{dq}^{m}C_{dr}A_{dr}TₖₛTₖₛAₖ | 5 |
| 1237988 | Gₖₛ^{m}CₖₛAₖₛT_{dq}G_{dq}T_{dq}T_{dq}^{m}C_{dq}T_{dq}^{m}C_{dr}A_{dr}^{m}C_{dq}A_{dq}TₖₛTₖₛAₖ | 5 |
| 1237989 | Gₖₛ^{m}CₖₛAₖₛT_{dq}G_{dq}T_{dq}T_{dq}^{m}C_{dr}T_{dr}^{m}C_{dq}A_{dq}^{m}C_{dq}A_{dq}TₖₛTₖₛAₖ | 5 |
| 1237990 | Gₖₛ^{m}CₖₛAₖₛT_{dq}G_{dq}T_{dr}T_{dr}^{m}C_{dq}T_{dq}^{m}C_{dq}A_{dq}^{m}C_{dq}A_{dq}TₖₛTₖₛAₖ | 5 |
| 1237991 | Gₖₛ^{m}CₖₛAₖₛT_{dr}G_{dr}T_{dq}T_{dq}^{m}C_{dq}T_{dq}^{m}C_{dq}A_{dq}^{m}C_{dq}A_{dq}TₖₛTₖₛAₖ | 5 |
| 1220052 | Gₖₛ^{m}CₖₛAₖₛT_{dr}G_{dr}T_{dr}T_{dr}^{m}C_{dr}T_{dr}^{m}C_{dr}A_{dr}^{m}C_{dr}A_{dr}TₖₛTₖₛAₖ | 5 |
| 1220053 | Gₖₛ^{m}CₖₛAₖₛT_{dq}G_{dr}T_{dr}T_{dr}^{m}C_{dr}T_{dr}^{m}C_{dr}A_{dr}^{m}C_{dr}A_{dr}TₖₛTₖₛAₖ | 5 |
| 1220054 | Gₖₛ^{m}CₖₛAₖₛT_{dr}G_{dq}T_{dr}T_{dr}^{m}C_{dr}T_{dr}^{m}C_{dr}A_{dr}^{m}C_{dr}A_{dr}TₖₛTₖₛAₖ | 5 |
| 1220055 | Gₖₛ^{m}CₖₛAₖₛT_{dr}G_{dr}T_{dq}T_{dr}^{m}C_{dr}T_{dr}^{m}C_{dr}A_{dr}^{m}C_{dr}A_{dr}TₖₛTₖₛAₖ | 5 |
| 1220056 | Gₖₛ^{m}CₖₛAₖₛT_{dr}G_{dr}T_{dr}T_{dq}^{m}C_{dr}T_{dr}^{m}C_{dr}A_{dr}^{m}C_{dr}A_{dr}TₖₛTₖₛAₖ | 5 |
| 1220057 | Gₖₛ^{m}CₖₛAₖₛT_{dr}G_{dr}T_{dr}T_{dr}^{m}C_{dq}T_{dr}^{m}C_{dr}A_{dr}^{m}C_{dr}A_{dr}TₖₛTₖₛAₖ | 5 |
| 1220058 | Gₖₛ^{m}CₖₛAₖₛT_{dr}G_{dr}T_{dr}T_{dr}^{m}C_{dr}T_{dq}^{m}C_{dr}A_{dr}^{m}C_{dr}A_{dr}TₖₛTₖₛAₖ | 5 |
| 1220059 | Gₖₛ^{m}CₖₛAₖₛT_{dr}G_{dr}T_{dr}T_{dr}^{m}C_{dr}T_{dr}^{m}T_{dq}A_{dr}^{m}C_{dr}A_{dr}TₖₛTₖₛAₖ | 5 |
| 1220060 | Gₖₛ^{m}CₖₛAₖₛT_{dr}G_{dr}T_{dr}T_{dr}^{m}C_{dr}T_{dr}^{m}C_{dr}A_{dq}^{m}C_{dr}A_{dr}TₖₛTₖₛAₖ | 5 |
| 1220061 | Gₖₛ^{m}CₖₛAₖₛT_{dr}G_{dr}T_{dr}T_{dr}^{m}C_{dr}T_{dr}^{m}C_{dr}A_{dr}^{m}C_{dq}A_{dr}TₖₛTₖₛAₖ | 5 |
| 1220062 | Gₖₛ^{m}CₖₛAₖₛT_{dr}G_{dr}T_{dr}T_{dr}^{m}C_{dr}T_{dr}^{m}C_{dr}A_{dr}^{m}C_{dr}A_{dq}TₖₛTₖₛAₖ | 5 |
| 1220063 | Gₖₛ^{m}CₖₛAₖₛT_{dq}G_{dq}T_{dr}T_{dq}^{m}C_{dq}T_{dr}^{m}C_{dq}A_{dq}^{m}C_{dr}A_{dr}TₖₛTₖₛAₖ | 5 |
| 1220064 | Gₖₛ^{m}CₖₛAₖₛT_{dr}G_{dr}T_{dq}T_{dr}^{m}C_{dr}T_{dq}^{m}C_{dr}A_{dr}^{m}C_{dq}A_{dq}TₖₛTₖₛAₖ | 5 |

A subscript “d” indicates an unmodified, 2′-deoxy sugar moiety. A subscript “k” indicates a cEt modified sugar moiety. A superscript “m” indicates 5-methyl Cytosine. A subscript “s” indicates a stereorandom phosphorothioate intemucleoside linkage, a subscript “r” indicates a phosphorothioate intemucleoside linkage having the (Rp) configuration, and a subscript “q” indicates a phosphorothioate intemucleoside linkage having the (Sp) configuration.

For in vitro activity studies, 3T3-L1 cells were plated at 20,000 cells/well and transfected with 27 nM, 80 nM, 250 nM, 740 nM, 2,222 nM, 6,667 nM, or 20,000 nMnM modified oligonucleotide by electroporation. mRNA was harvested and analyzed by RT-qPCR. CXCL12 mRNA was detected with primer probe set RTS 2605 (forward sequence CCAGAGCCAACGTCAAGCAT, SEQ ID NO: 2; reverse sequence: CAGCCGTGCAACAATCTGAA, SEQ ID NO: 3; probe sequence:

TGAAAATCCTCAACACTCCAAACTGTGCC, SEQ ID NO: 4) and P21 mRNA was detected with primer probe set Mm04207341_ml (ThermoFisher).

Caspase-3 and caspase-7 activation was measured using the Caspase-Glo® 3/7 Assay System (G8090, Promega). Levels of caspase activation correlate with apoptotic cell death. Results are presented relative to the caspase activation in control cells not treated with modified oligonucleotide. Localization of p54nrb in HeLa cells was visualized with confocal microscopy. HeLa cells were transfected by lipofectamine 2000 with 200 nM of modified oligonucleotide for 2 hrs and then cellular protein p54nrb was stained by mP54 antibody (Santa Cruz Biotech, sc-376865) and DAPI was used to stain for the nucleus of cells. The number of cells with nucleolar p54nrb and the total number of cells in the images were counted. The self-structure Tm of each compound was determined.

TABLE 2
In vitro activity, toxicity, and Tm of modified
oligonucleotides complementary to CXCL12

| | in vitro | in vitro | | | |
|---|---|---|---|---|---|
| | CXCL12 | Caspase | P21 mRNA | % | |
| Compound | IC₅₀ | (% control) | (% control) | nucleolar | Tm |
| ID | (nM) | @ 20 μM | @ 20 μM | p54nrb | (° C.) |
| 558807 | 39 | 1437 | 353 | 90 | 64.4 |
| 1220041 | 388 | 223 | 182 | 0 | 61.3 |
| 1220042 | 159 | 584 | 431 | 32 | 62.1 |
| 1220043 | 114 | 838 | 488 | 88 | 62 |
| 1220044 | 181 | 489 | 251 | 18 | 61.5 |
| 1220045 | 222 | 321 | 259 | 9.7 | 61.9 |
| 1220046 | 145 | 572 | 635 | 28 | 61.7 |
| 1220051 | 237 | 310 | 167 | 20 | 61.6 |
| 1220047 | 60 | 814 | 238 | 38 | 61.5 |
| 1220048 | 74 | 287 | 174 | 38 | 61.3 |
| 1220049 | 77 | 323 | 243 | 17 | 61.6 |
| 1220050 | 132 | 174 | 121 | 6.4 | 61.5 |
| 1237987 | 26 | 317 | 273 | 3.9 | 62.2 |
| 1237988 | 20 | 336 | 236 | 23 | 62.1 |
| 1237989 | 72 | 300 | 394 | 28 | 62.2 |
| 1237990 | 186 | 299 | 355 | 14 | 62.5 |
| 1237991 | 35 | 562 | 585 | 77 | 63 |

TABLE 3
In vitro activity, toxicity, and Tm of modified
oligonucleotides complementary to CXCL12

| | in vitro | in vitro | | | |
|---|---|---|---|---|---|
| | CXCL12 | Caspase | P21 mRNA | % | |
| Compound | IC₅₀ | (% control) | (% control) | nucleolar | |
| ID | (nM) | @ 20 μM | @ 20 μM | p54nrb | Tm |
| 558807 | 95 | 647 | 235 | 93 | 64.4 |
| 1220052 | 63 | 484 | 272 | 98 | 67.4 |
| 1220053 | 99 | 621 | 261 | 95 | 66.2 |
| 1220054 | 197 | 495 | 192 | 96 | 66.8 |
| 1220055 | 51 | 606 | 370 | 100 | 66.9 |
| 1220056 | 103 | 569 | 369 | 97 | 67 |
| 1220057 | 104 | 593 | 330 | 92 | 67.1 |
| 1220058 | 125 | 578 | 273 | 100 | 67.3 |
| 1220059 | 109 | 525 | 351 | 62 | 66.7 |
| 1220060 | 61 | 553 | 328 | 100 | 67.3 |
| 1220061 | 84 | 409 | 329 | 100 | 67.1 |
| 1220062 | 123 | 550 | 394 | 100 | 67.1 |
| 1220063 | 111 | 138 | 128 | 12 | 63.1 |
| 1220064 | 53 | 160 | 218 | 100 | 65.3 |

### Example 2. Nucleosides with Chiral Phosphorothioate Linkages and 3′-GalNAc

Modified oligonucleotides containing chirally-controlled phosphorothioate linkages in the central region and a 3′-GalNAc were synthesized. The compounds in Table 4 are 100% complementary to mouse CXCL12, GENBANK NT 039353.7 truncated from 69430515 to 69445350 (SEQ ID NO: 1), at position 6877 to 6892. Each compound has the kkk-d(10)-kkk sugar motif, wherein each “k” represents a 2′-constrained ethyl modified sugar moiety and each “d” represents a 2′-deoxy sugar moiety. Internucleoside linkages 1, 2, 3, 14, and 15 are stereorandom phosphorothioate linkages. Internucleoside linkages 4-13 have the stereochemistry indicated in the table below, wherein a subscript “s” indicates a stereorandom phosphorothioate internucleoside linkage, a subscript “r” indicates a phosphorothioate internucleoside linkage having the (Rp) configuration and a subscript “q” indicates a phosphorothioate internucleoside linkage having the (Sp) configuration.
GalNAc refers to this structure at the 3′ end of the molecule:

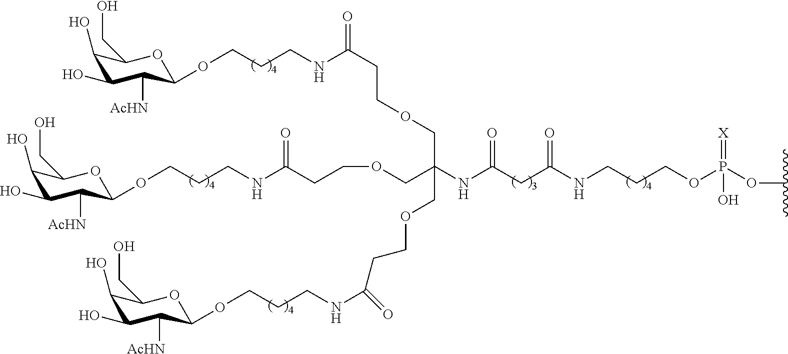

TABLE 4
Modified oligonucleotides

| Compound | | SEQ |
|---|---|---|
| ID | Chemistry Notation | ID NO |
| 558807 | Gₖₛ^{m}CₖₛAₖₛT_{ds}G_{ds}T_{ds}T_{ds}^{m}C_{ds}T_{ds}^{m}C_{ds}A_{ds}^{m}C_{ds}A_{ds}TₖₛT_{ds}Aₖ | 5 |
| 855156 | Gₖₛ^{m}CₖₛAₖₛT_{ds}G_{ds}T_{ds}T_{ds}^{m}C_{ds}T_{ds}^{m}C_{ds}A_{ds}^{m}C_{ds}A_{ds}TₖₛTₖₛAₖ-GalNAc | 5 |
| 1220050 | Gₖₛ^{m}CₖₛAₖₛT_{dq}G_{dq}T_{dq}T_{dq}^{m}C_{dq}T_{dq}^{m}C_{dq}A_{dq}^{m}C_{dq}A_{dr}TₖₛTₖₛAₖ | 5 |
| 1277251 | Gₖₛ^{m}CₖₛAₖₛT_{dq}G_{dq}T_{dq}T_{dq}^{m}C_{dq}T_{dq}^{m}C_{dq}A_{dq}^{m}C_{dq}A_{dr}TₖₛTₖₛAₖ-GalNAc | 5 |
| 1220059 | Gₖₛ^{m}CₖₛAₖₛT_{dr}G_{dr}T_{dr}T_{dr}^{m}C_{dr}T_{dr}^{m}C_{dq}A_{dr}^{m}C_{dr}A_{dr}TₖₛTₖₛAₖ | 5 |
| 1277252 | Gₖₛ^{m}CₖₛAₖₛT_{dr}G_{dr}T_{dr}T_{dr}^{m}C_{dr}T_{dr}^{m}C_{dq}A_{dr}^{m}C_{dr}A_{dr}TₖₛTₖₛAₖ-GalNAc | 5 |
| 1220063 | Gₖₛ^{m}CₖₛAₖₛT_{dq}G_{dq}T_{dr}T_{dq}^{m}C_{dq}T_{dr}^{m}C_{dq}A_{dq}^{m}C_{dr}A_{dr}TₖₛTₖₛAₖ | 5 |
| 1277253 | Gₖₛ^{m}CₖₛAₖₛT_{dq}G_{dq}T_{dr}T_{dq}^{m}C_{dq}T_{dr}^{m}C_{dq}A_{dq}^{m}C_{dr}A_{dr}TₖₛTₖₛAₖ-GalNAc | 5 |
| 1237988 | Gₖₛ^{m}CₖₛAₖₛT_{dq}G_{dq}T_{dq}T_{dq}^{m}C_{dq}T_{dq}^{m}C_{dr}A_{dr}^{m}C_{dq}A_{dr}TₖₛAₖ | 5 |
| 1277254 | Gₖₛ^{m}CₖₛAₖₛT_{dq}G_{dq}T_{dq}T_{dq}^{m}C_{dq}T_{dq}^{m}C_{dr}A_{dr}^{m}C_{dq}A_{dr}TₖₛTₖₛAₖ-GalNAc | 5 |

A subscript “d” indicates an unmodified, 2′-deoxy sugar moiety. A subscript “k” indicates a cEt modified sugar moiety. A superscript “m” indicates 5-methyl Cytosine. A subscript “s” indicates a stereorandom phosphorothioate internucleoside linkage, a subscript “r” indicates a phosphorothioate internucleoside linkage haying the (Rp) configuration, and a subscript “q” indicates a phosphorothioate internucleoside linkage haying the (Sp) configuration.

TABLE 5
In vitro activity and toxicity of modified
oligonucleotides complementary to CXCL12

| | in vitro | in vitro | |
|---|---|---|---|
| Compound | CXCL12 IC₅₀ | Caspase (% control) | % nucleolar |
| ID | (nM) | @ 20 μM | p54nrb |
| 855156 | 40 | 1437 | 90 |
| 1277251 | 130 | 174 | 6.4 |
| 1277252 | 111 | 525 | 62 |
| 1277253 | 111 | 138 | 12 |
| 1277254 | 20 | 336 | 24 |

### Example 3. Nucleosides with Two Chiral Phosphate Linkages in an Otherwise Stereorandom Phosphorothioate Nucleotide

Modified oligonucleotides containing chirally-controlled phosphorothioate linkages at two positions of the central region were synthesized. The compounds in Table 6 are 100% complementary to mouse CXCL12, GENBANK NT_039353.7 truncated from 69430515 to 69445350 (SEQ ID NO: 1), at position 6877 to 6892. Each compound with an ID in the range of 1273959-1273967 has a kkk-d(10)-kkk sugar motif, wherein each “k” represents a 2′-constrained ethyl modified sugar moiety and each “d” represents a 2′-deoxy sugar moiety. Each compound with an ID in the range of 1276491-1276497 has a kkk-d-m-d(8)-kkk sugar motif, wherein each “k” represents a 2′-constrained ethyl modified sugar moiety and each “d” represents a 2′-deoxy sugar moiety and “m” represents a 2′-Omethyl modified sugar moiety. Internucleoside linkages are as indicated in the table below, wherein a subscript “s” indicates a stereorandom phosphorothioate internucleoside linkage, a subscript “r” indicates a phosphorothioate internucleoside linkage having the (Rp) configuration and a subscript “q” indicates a phosphorothioate internucleoside linkage having the (Sp) configuration. Each compound contains an “Rp/Sp” unit comprising an internucleoside linkage having the (Rp) configuration followed by an internucleoside linkage having the (Sp) configuration, from 5′-3′.

Compounds were tested in 3T3-L1 cells for caspase activation as described in Example 1 above.

TABLE 6
Modified oligonucleotides

| Compound | | |
|---|---|---|
| ID | Chemistry Notation | SEQ ID NO |
| 1273959 | Gₖₛ^{m}CₖₛAₖₛT_{dr}G_{dq}T_{ds}T_{ds}^{m}C_{ds}T_{ds}^{m}C_{ds}A_{ds}^{m}C_{ds}A_{ds}TₖₛT_{ds}Aₖ | 5 |
| 1273960 | Gₖₛ^{m}CₖₛAₖₛT_{ds}G_{dr}T_{dq}T_{ds}^{m}C_{ds}T_{ds}^{m}C_{ds}A_{ds}^{m}C_{ds}A_{ds}TₖₛT_{ds}Aₖ | 5 |
| 1273961 | Gₖₛ^{m}CₖₛAₖₛT_{ds}G_{ds}T_{dr}T_{dq}^{m}C_{ds}T_{ds}^{m}C_{ds}A_{ds}^{m}C_{ds}A_{ds}TₖₛT_{ds}Aₖ | 5 |
| 1273962 | Gₖₛ^{m}CₖₛAₖₛT_{ds}G_{ds}T_{ds}T_{dr}^{m}C_{dq}T_{ds}^{m}C_{ds}A_{ds}^{m}C_{ds}A_{ds}TₖₛT_{ds}Aₖ | 5 |
| 1273963 | Gₖₛ^{m}CₖₛAₖₛT_{ds}G_{ds}T_{ds}T_{ds}^{m}C_{dr}T_{dq}^{m}C_{ds}A_{ds}^{m}C_{ds}A_{ds}TₖₛT_{ds}Aₖ | 5 |
| 1273964 | Gₖₛ^{m}CₖₛAₖₛT_{ds}G_{ds}T_{ds}T_{ds}^{m}C_{ds}T_{dr}^{m}C_{dq}A_{ds}^{m}C_{ds}A_{ds}TₖₛT_{ds}Aₖ | 5 |
| 1273965 | Gₖₛ^{m}CₖₛAₖₛT_{ds}G_{ds}T_{ds}T_{ds}^{m}C_{ds}T_{ds}^{m}C_{dr}A_{dq}^{m}C_{ds}A_{ds}TₖₛT_{ds}Aₖ | 5 |
| 1273966 | Gₖₛ^{m}CₖₛAₖₛT_{ds}G_{ds}T_{ds}T_{ds}^{m}C_{ds}T_{ds}^{m}C_{ds}A_{dr}^{m}C_{dq}A_{ds}TₖₛTₖₛAₖ | 5 |
| 1273967 | Gₖₛ^{m}CₖₛAₖₛT_{ds}G_{ds}T_{ds}T_{ds}^{m}C_{ds}T_{ds}^{m}C_{ds}A_{ds}^{m}C_{dr}A_{dq}TₖₛT_{ds}Aₖ | 5 |
| 1276491 | Gₖₛ^{m}CₖₛAₖₛT_{ds}GₘₛT_{dr}T_{dq}^{m}C_{ds}T_{ds}^{m}C_{ds}A_{ds}^{m}C_{ds}A_{ds}TₖₛT_{ds}Aₖ | 5 |
| 1276492 | Gₖₛ^{m}CₖₛAₖₛT_{ds}GₘₛT_{ds}T_{dr}^{m}C_{dq}T_{ds}^{m}C_{ds}A_{ds}^{m}C_{ds}A_{ds}TₖₛT_{ds}Aₖ | 5 |
| 1276493 | Gₖₛ^{m}CₖₛAₖₛT_{ds}GₘₛT_{ds}T_{ds}^{m}C_{dr}T_{dq}^{m}C_{ds}A_{ds}^{m}C_{ds}A_{ds}TₖₛT_{ds}Aₖ | 5 |
| 1276494 | Gₖₛ^{m}CₖₛAₖₛT_{ds}GₘₛT_{ds}T_{ds}^{m}C_{ds}T_{dr}^{m}C_{dq}A_{ds}^{m}C_{ds}A_{ds}TₖₛT_{ds}Aₖ | 5 |
| 1276495 | Gₖₛ^{m}CₖₛAₖₛT_{ds}GₘₛT_{ds}T_{ds}^{m}C_{ds}T_{ds}^{m}C_{dr}A_{dq}^{m}C_{ds}A_{ds}TₖₛT_{ds}Aₖ | 5 |
| 1276496 | Gₖₛ^{m}CₖₛAₖₛT_{ds}GₘₛT_{ds}T_{ds}^{m}C_{ds}T_{ds}^{m}C_{ds}A_{dr}^{m}C_{dq}A_{ds}TₖₛT_{ds}Aₖ | 5 |
| 1276497 | Gₖₛ^{m}CₖₛAₖₛT_{ds}GₘₛT_{ds}T_{ds}^{m}C_{ds}T_{ds}^{m}C_{ds}A_{ds}^{m}C_{dr}A_{dq}TₖₛT_{ds}Aₖ | 5 |

A subscript “d” indicates an unmodified, 2′-deoxy sugar moiety. A subscript “k” indicates cEt modified sugar moiety. A superscript “m” indicates 5-methyl Cytosine. A subscript “s” indicates a stereorandom phosphorothioate internucleoside linkage, a subscript “r” indicates a phosphorothioate internucleoside linkage having the (Rp) configuration, and a subscript “q” indicates a phosphorothioate internucleoside linkage having the (Sp) configuration. A subscript “m” represents a 2′-Omethyl modified sugar moiety.

TABLE 7
In vitro toxicity of modified oligonucleotides
complementary to CXCL12

| Compound | in vitro Caspase |
|---|---|
| ID | (% control) @ 20 μM |
| 1273959 | 1138 |
| 1273960 | 654 |
| 1273961 | 1036 |
| 1273962 | 752 |
| 1273963 | 1349 |
| 1273964 | 907 |
| 1273965 | 984 |
| 1273966 | 750 |
| 1273967 | 785 |
| 1276491 | 116 |
| 1276492 | 450 |
| 1276493 | 234 |
| 1276494 | 85 |
| 1276495 | 214 |
| 1276496 | 165 |
| 1276497 | 148 |

What is claimed:

1. A chirally enriched population of oligomeric compounds comprising modified oligonucleotides, wherein the modified oligonucleotides consist of 12-23 linked nucleosides, wherein the modified oligonucleotide comprises a gapmer consisting of a 5′-region, a central region, and a 3′-region wherein:
the 5′-region consists of 1-5 linked modified nucleosides, wherein each nucleoside of the 5′-region comprises a 2′-modified furanosyl sugar moiety;
the 3′-region consists of 1-5 linked modified nucleosides, wherein each nucleoside of the 3′-region comprises a 2′-modified furanosyl sugar moiety;
the central region consists of 7-10 linked nucleosides, where each nucleoside of the central region comprises a 2′-β-D-deoxyribosyl sugar moiety; and
wherein the central region has at least one phosphorothioate internucleoside linkage that is chirally enriched in the (Sp) configuration or the (Rp) configuration.

2. A chirally enriched population of oligomeric compounds, wherein the oligomeric compounds comprise a modified oligonucleotide consisting of 12-23 linked nucleosides, wherein the modified oligonucleotide is a gapmer consisting of a 5′-region, a central region, and a 3′-region wherein:
the 5′-region consists of 1-5 linked modified nucleosides, wherein each nucleoside of the 5′-region comprises a 2′-modified furanosyl sugar moiety;
the 3′-region consists of 1-5 linked modified nucleosides, wherein each nucleoside of the 3′-region comprises a 2′-modified furanosyl sugar moiety;
the central region consists of 7-10 linked nucleosides, where each nucleoside of the central region comprises a 2′-β-D-deoxyribosyl sugar moiety;
wherein the central region has at least one phosphorothioate internucleoside linkage; and
wherein a percentage of oligomeric compounds within the population that contain a stereochemical configuration at the phosphorothioate internucleoside linkage is greater than an expected percentage of oligomeric compounds expected to contain the same particular stereochemical configuration at the phosphorothioate internucleoside linkage within the population if the stereochemical configuration at the phosphorothioate internucleoside linkage was stereorandom.

3. The chirally enriched population of oligomeric compounds of claim 1 or 2, wherein each central region internucleoside linkage is selected from among a phosphodiester internucleoside linkage and a phosphorothioate internucleoside linkage.

4. The chirally enriched population of oligomeric compounds of claim 1 or 2, wherein the central region has 2 phosphorothioate internucleoside linkages.

5. The chirally enriched population of oligomeric compounds of claim 1 or 2, wherein the central region has 3 phosphorothioate internucleoside linkages.

6. The chirally enriched population of oligomeric compounds of claim 1 or 2, wherein the central region has 4 phosphorothioate internucleoside linkages.

7. The chirally enriched population of oligomeric compounds of claim 1 or 2, wherein the central region has 5 phosphorothioate internucleoside linkages.

8. The chirally enriched population of oligomeric compounds of claim 1 or 2, wherein the central region has 6 phosphorothioate internucleoside linkages.

9. The chirally enriched population of oligomeric compounds of claim 1 or 2, wherein the central region has 7 phosphorothioate internucleoside linkages.

10. The chirally enriched population of oligomeric compounds of claim 1 or 2, wherein the central region has 8 phosphorothioate internucleoside linkages.

11. The chirally enriched population of oligomeric compounds of claim 1 or 2, wherein the central region has 9 phosphorothioate internucleoside linkages.

12. The chirally enriched population of oligomeric compounds of claim 1 or 2, wherein the central region has 10 phosphorothioate internucleoside linkages.

13. The chirally enriched population of oligomeric compounds of claim 1 or 2, wherein the central region has 11 phosphorothioate internucleoside linkages.

14. The chirally enriched population of oligomeric compounds of claim 1 or 2, wherein each central region internucleoside linkage is a phosphorothioate internucleoside linkage.

15. The chirally enriched population of oligomeric compounds of claim 1 or 2, wherein each phosphorothioate internucleoside linkage has the (Sp) configuration.

16. The chirally enriched population of oligomeric compounds of claim 1 or 2, wherein each phosphorothioate internucleoside linkage has the (Rp) configuration.

17. The chirally enriched population of oligomeric compounds of claim 1 or 2, wherein 1 central region internucleoside linkage has the (Rp) configuration.

18. The chirally enriched population of oligomeric compounds of any of claims 1-14, wherein 2 central region internucleoside linkages have the (Rp) configuration.

19. The chirally enriched population of oligomeric compounds of claim 18, wherein the 2 central region internucleoside linkages having the (Rp) configuration are adjacent.

20. The chirally enriched population of oligomeric compounds of claim 1 or 2, wherein the central region consists of 7 linked nucleosides and has the formula:
(N_{d})_{L1}(N_{d})_{L2}(N_{d})_{L3}(N_{d})_{L4}(N_{d})_{L5}(N_{d})_{L6}(N_{d})_{L7}; wherein
each N_{d} is a 2′-β-D-deoxyribosyl sugar moiety and each of L1, L2, L3, L4, L5, L6, and L7 is a phosphorothioate internucleoside linkage; and
wherein at least one of L1, L2, L3, L4, L5, L6, and L7 has the (Sp) configuration or the (Rp) configuration.

21. The chirally enriched population of oligomeric compounds of claim 20, wherein L1 is a phosphorothioate internucleoside linkage having the (Rp) configuration and where each of L2, L3, L4, L5, L6, and L7 is a phosphorothioate internucleoside linkage having the (Sp) configuration.

22. The chirally enriched population of oligomeric compounds of claim 20, wherein L₂ is a phosphorothioate internucleoside linkage having the (Rp) configuration and where each of L1, L3, L4, L5, L6, and L7 is a phosphorothioate internucleoside linkage having the (Sp) configuration.

23. The chirally enriched population of oligomeric compounds of claim 20, wherein L3 is a phosphorothioate internucleoside linkage having the (Rp) configuration and where each of L1, L2, L4, L5, L6, and L7 is a phosphorothioate internucleoside linkage having the (Sp) configuration.

24. The chirally enriched population of oligomeric compounds of claim 20, wherein L4 is a phosphorothioate internucleoside linkage having the (Rp) configuration and where each of L1, L2, L3, L5, L6, and L7 is a phosphorothioate internucleoside linkage having the (Sp) configuration.

25. The chirally enriched population of oligomeric compounds of claim 20, wherein L5 is a phosphorothioate internucleoside linkage having the (Rp) configuration and where each of L1, L2, L3, L4, L6, and L7 is a phosphorothioate internucleoside linkage having the (Sp) configuration.

26. The chirally enriched population of oligomeric compounds of claim 20, wherein L6 is a phosphorothioate internucleoside linkage having the (Rp) configuration and where each of L1, L2, L3, L4, L5, and L7 is a phosphorothioate internucleoside linkage having the (Sp) configuration.

27. The chirally enriched population of oligomeric compounds of claim 20, wherein L7 is a phosphorothioate internucleoside linkage having the (Rp) configuration and where each of L1, L2, L3, L4, L5, and L6 is a phosphorothioate internucleoside linkage having the (Sp) configuration.

28. The chirally enriched population of oligomeric compounds of claim 20, wherein each L1 and L₂ is a phosphorothioate internucleoside linkage having the (Rp) configuration and where each of L3, L4, L5, L6, and L7 is a phosphorothioate internucleoside linkage having the (Sp) configuration.

29. The chirally enriched population of oligomeric compounds of claim 20, wherein each L₂ and L3 is a phosphorothioate internucleoside linkage having the (Rp) configuration and where each of L1, L4, L5, L6, and L7 is a phosphorothioate internucleoside linkage having the (Sp) configuration.

30. The chirally enriched population of oligomeric compounds of claim 20, wherein each L3 and L4 is a phosphorothioate internucleoside linkage having the (Rp) configuration and where each of L1, L2, L5, L6, and L7 is a phosphorothioate internucleoside linkage having the (Sp) configuration.

31. The chirally enriched population of oligomeric compounds of claim 20, wherein each L4 and L5 is a phosphorothioate internucleoside linkage having the (Rp) configuration and where each of L1, L2, L3, L6, and L7 is a phosphorothioate internucleoside linkage having the (Sp) configuration.

32. The chirally enriched population of oligomeric compounds of claim 20, wherein each L5 and L6 is a phosphorothioate internucleoside linkage having the (Rp) configuration and where each of L1, L2, L3, L4, and L7 is a phosphorothioate internucleoside linkage having the (Sp) configuration.

33. The chirally enriched population of oligomeric compounds of claim 20, wherein each L6 and L7 is a phosphorothioate internucleoside linkage having the (Rp) configuration and where each of L1, L2, L3, L4, and L5 is a phosphorothioate internucleoside linkage having the (Sp) configuration.

34. The chirally enriched population of oligomeric compounds of claim 20, wherein each L1 is a phosphorothioate internucleoside linkage having the (Rp) configuration, each L₂ is a phosphorothioate internucleoside linkage having the (Sp) configuration, and where each of L3, L4, L5, L6 and L7 is a stereorandom phosphorothioate internucleoside linkage.

35. The chirally enriched population of oligomeric compounds of claim 20, wherein each L₂ is a phosphorothioate internucleoside linkage having the (Rp) configuration, each L3 is a phosphorothioate internucleoside linkage having the (Sp) configuration, and where each of L1, L4, L5, L6 and L7 is a stereorandom phosphorothioate internucleoside linkage.

36. The chirally enriched population of oligomeric compounds of claim 20, wherein each L3 is a phosphorothioate internucleoside linkage having the (Rp) configuration, each L4 is a phosphorothioate internucleoside linkage having the (Sp) configuration, and where each of L1, L2, L5, L6 and L7 is a stereorandom phosphorothioate internucleoside linkage.

37. The chirally enriched population of oligomeric compounds of claim 20, wherein each L4 is a phosphorothioate internucleoside linkage having the (Rp) configuration, each L5 is a phosphorothioate internucleoside linkage having the (Sp) configuration, and where each of L1, L2, L3, L6 and L7 is a stereorandom phosphorothioate internucleoside linkage.

38. The chirally enriched population of oligomeric compounds of claim 20, wherein each L5 is a phosphorothioate internucleoside linkage having the (Rp) configuration, each L6 is a phosphorothioate internucleoside linkage having the (Sp) configuration, and where each of L1, L2, L3, L4 and L7 is a stereorandom phosphorothioate internucleoside linkage.

39. The chirally enriched population of oligomeric compounds of claim 20, wherein each L6 is a phosphorothioate internucleoside linkage having the (Rp) configuration, each L7 is a phosphorothioate internucleoside linkage having the (Sp) configuration, and where each of L1, L2, L3, L4 and L5 is a stereorandom phosphorothioate internucleoside linkage.

40. The oligomeric compound of claim 1 or 2, wherein the central region consists of 8 linked nucleosides and has the formula:
(N_{d})_{L1}(N_{d})_{L2}(N_{d})_{L3}(N_{d})_{L4}(N_{d})_{L5}(N_{d})_{L6}(N_{d})_{L7}(N_{d})_{L8}; wherein
each N_{d} is a 2′-β-D-deoxyribosyl sugar moiety and each of L1, L2, L3, L4, L5, L6, L7, and L8 is a phosphorothioate internucleoside linkage; and
wherein at least one of L1, L2, L3, L4, L5, L6, L7 and L8 has the (Sp) configuration or the (Rp) configuration.

41. The oligomeric compound of claim 1 or 2, wherein the central region consists of 9 linked nucleosides and has the formula:
(N_{d})_{L1}(N_{d})_{L2}(N_{d})_{L3}(N_{d})_{L4}(N_{d})_{L5}(N_{d})_{L6}(N_{d})_{L7}(N_{d})_{L8}(N_{d})_{L9}; wherein
each N_{d} is a 2′-β-D-deoxyribosyl sugar moiety and each of L1, L2, L3, L4, L5, L6, L7, L8, and L9 is a phosphorothioate internucleoside linkage; and
wherein at least one of L1, L2, L3, L4, L5, L6, L7, L8 and L9 has the (Sp) configuration or the (Rp) configuration.

42. The chirally enriched population of oligomeric compounds of claim 41, wherein L1 is a phosphorothioate internucleoside linkage having the (Rp) configuration and where each of L2, L3, L4, L5, L6, L7 and L8 is a phosphorothioate internucleoside linkage having the (Sp) configuration.

43. The chirally enriched population of oligomeric compounds of claim 41, wherein L₂ is a phosphorothioate internucleoside linkage having the (Rp) configuration and where each of L1, L3, L4, L5, L6, L7 and L8 is a phosphorothioate internucleoside linkage having the (Sp) configuration.

44. The chirally enriched population of oligomeric compounds of claim 41, wherein L3 is a phosphorothioate internucleoside linkage having the (Rp) configuration and where each of L1, L2, L4, L5, L6, L7 and L8 is a phosphorothioate internucleoside linkage having the (Sp) configuration.

45. The chirally enriched population of oligomeric compounds of claim 41, wherein L4 is a phosphorothioate internucleoside linkage having the (Rp) configuration and where each of L1, L2, L4, L5, L6, L7 and L8 is a phosphorothioate internucleoside linkage having the (Sp) configuration.

46. The chirally enriched population of oligomeric compounds of claim 41, wherein L5 is a phosphorothioate internucleoside linkage having the (Rp) configuration and where each of L1, L2, L3, L4, L6, L7 and L8 is a phosphorothioate internucleoside linkage having the (Sp) configuration.

47. The chirally enriched population of oligomeric compounds of claim 41, wherein L6 is a phosphorothioate internucleoside linkage having the (Rp) configuration and where each of L1, L2, L3, L4, L5, L7 and L8 is a phosphorothioate internucleoside linkage having the (Sp) configuration.

48. The chirally enriched population of oligomeric compounds of claim 41, wherein L7 is a phosphorothioate internucleoside linkage having the (Rp) configuration and where each of L1, L2, L3, L4, L5, L6 and L8 is a phosphorothioate internucleoside linkage having the (Sp) configuration.

49. The chirally enriched population of oligomeric compounds of claim 41, wherein L8 is a phosphorothioate internucleoside linkage having the (Rp) configuration and where each of L1, L2, L3, L4, L5, L6 and L7 is a phosphorothioate internucleoside linkage having the (Sp) configuration.

50. The chirally enriched population of oligomeric compounds of claim 41, wherein each L1 and L₂ is a phosphorothioate internucleoside linkage having the (Rp) configuration and where each of L3, L4, L5, L6 and L8 is a phosphorothioate internucleoside linkage having the (Sp) configuration.

51. The chirally enriched population of oligomeric compounds of claim 41, wherein each L₂ and L3 is a phosphorothioate internucleoside linkage having the (Rp) configuration and where each of L1, L4, L5, L6 and L8 is a phosphorothioate internucleoside linkage having the (Sp) configuration.

52. The chirally enriched population of oligomeric compounds of claim 41, wherein each L3 and L4 is a phosphorothioate internucleoside linkage having the (Rp) configuration and where each of L1, L2, L5, L6 and L8 is a phosphorothioate internucleoside linkage having the (Sp) configuration.

53. The chirally enriched population of oligomeric compounds of claim 41, wherein each L4 and L5 is a phosphorothioate internucleoside linkage having the (Rp) configuration and where each of L1, L2, L3, L6 and L8 is a phosphorothioate internucleoside linkage having the (Sp) configuration.

54. The chirally enriched population of oligomeric compounds of claim 41, wherein each L5 and L6 is a phosphorothioate internucleoside linkage having the (Rp) configuration and where each of L1, L2, L3, L6 and L8 is a phosphorothioate internucleoside linkage having the (Sp) configuration.

55. The chirally enriched population of oligomeric compounds of claim 41, wherein each L6 and L7 is a phosphorothioate internucleoside linkage having the (Rp) configuration and where each of L1, L2, L3, L5 and L8 is a phosphorothioate internucleoside linkage having the (Sp) configuration.

56. The chirally enriched population of oligomeric compounds of claim 41, wherein each L7 and L8 is a phosphorothioate internucleoside linkage having the (Rp) configuration and where each of L1, L2, L3, L5 and L6 is a phosphorothioate internucleoside linkage having the (Sp) configuration.

57. The chirally enriched population of oligomeric compounds of claim 41, wherein each L1 is a phosphorothioate internucleoside linkage having the (Rp) configuration, each L₂ is a phosphorothioate internucleoside linkage having the (Sp) configuration, and where each of L3, L4, L5, L6, L7 and L8 is a stereorandom phosphorothioate internucleoside linkage.

58. The chirally enriched population of oligomeric compounds of claim 41, wherein each L₂ is a phosphorothioate internucleoside linkage having the (Rp) configuration, each L3 is a phosphorothioate internucleoside linkage having the (Sp) configuration, and where each of L1, L4, L5, L6, L7 and L8 is a stereorandom phosphorothioate internucleoside linkage.

59. The chirally enriched population of oligomeric compounds of claim 41, wherein each L3 is a phosphorothioate internucleoside linkage having the (Rp) configuration, each L4 is a phosphorothioate internucleoside linkage having the (Sp) configuration, and where each of L1, L2, L5, L6, L7 and L8 is a stereorandom phosphorothioate internucleoside linkage.

60. The chirally enriched population of oligomeric compounds of claim 41, wherein each L4 is a phosphorothioate internucleoside linkage having the (Rp) configuration, each L5 is a phosphorothioate internucleoside linkage having the (Sp) configuration, and where each of L1, L2, L3, L6, L7 and L8 is a stereorandom phosphorothioate internucleoside linkage.

61. The chirally enriched population of oligomeric compounds of claim 41, wherein each L5 is a phosphorothioate internucleoside linkage having the (Rp) configuration, each L6 is a phosphorothioate internucleoside linkage having the (Sp) configuration, and where each of L1, L2, L3, L4, L7 and L8 is a stereorandom phosphorothioate internucleoside linkage.

62. The chirally enriched population of oligomeric compounds of claim 41, wherein each L6 is a phosphorothioate internucleoside linkage having the (Rp) configuration, each L7 is a phosphorothioate internucleoside linkage having the (Sp) configuration, and where each of L1, L2, L3, L4, L5 and L8 is a stereorandom phosphorothioate internucleoside linkage.

63. The chirally enriched population of oligomeric compounds of claim 41, wherein each L7 is a phosphorothioate internucleoside linkage having the (Rp) configuration, each L8 is a phosphorothioate internucleoside linkage having the (Sp) configuration, and where each of L1, L2, L3, L4, L5 and L6 is a stereorandom phosphorothioate internucleoside linkage.

64. The oligomeric compound of claim 1 or 2, wherein the central region consists of 9 linked nucleosides and has the formula:
(N_{d})_{L1}(N_{d})_{L2}(N_{d})_{L3}(N_{d})_{L4}(N_{d})_{L5}(N_{d})_{L6}(N_{d})_{L7}(N_{d})_{L8}(N_{d})_{L9}; wherein
each N_{d} is a 2′-β-D-deoxyribosyl sugar moiety and each of L1, L2, L3, L4, L5, L6, L7, L8, and L9 is a phosphorothioate internucleoside linkage; and
wherein at least one of L1, L2, L3, L4, L5, L6, L7, L8 and L9 has the (Sp) configuration or the (Rp) configuration.

65. The chirally enriched population of oligomeric compounds of claim 51 or 64, wherein L1 is a phosphorothioate internucleoside linkage having the (Rp) configuration and where each of L2, L3, L4, L5, L6, L7, L8 and L9 is a phosphorothioate internucleoside linkage having the (Sp) configuration.

66. The chirally enriched population of oligomeric compounds of claim 64, wherein L₂ is a phosphorothioate internucleoside linkage having the (Rp) configuration and where each of L1, L3, L4, L5, L6, L7, L8 and L9 is a phosphorothioate internucleoside linkage having the (Sp) configuration.

67. The chirally enriched population of oligomeric compounds of claim 64, wherein L3 is a phosphorothioate internucleoside linkage having the (Rp) configuration and where each of L1, L2, L4, L5, L6, L7, L8 and L9 is a phosphorothioate internucleoside linkage having the (Sp) configuration.

68. The chirally enriched population of oligomeric compounds of claim 64, wherein L4 is a phosphorothioate internucleoside linkage having the (Rp) configuration and where each of L1, L2, L3, L5, L6, L7, L8 and L9 is a phosphorothioate internucleoside linkage having the (Sp) configuration.

69. The chirally enriched population of oligomeric compounds of claim 64, wherein L5 is a phosphorothioate internucleoside linkage having the (Rp) configuration and where each of L1, L2, L3, L4, L6, L7, L8 and L9 is a phosphorothioate internucleoside linkage having the (Sp) configuration.

70. The chirally enriched population of oligomeric compounds of claim 64, wherein L6 is a phosphorothioate internucleoside linkage having the (Rp) configuration and where each of L1, L2, L3, L4, L5, L7, L8 and L9 is a phosphorothioate internucleoside linkage having the (Sp) configuration.

71. The chirally enriched population of oligomeric compounds of claim 64, wherein L7 is a phosphorothioate internucleoside linkage having the (Rp) configuration and where each of L1, L2, L3, L4, L5, L6, L8 and L9 is a phosphorothioate internucleoside linkage having the (Sp) configuration.

72. The chirally enriched population of oligomeric compounds of claim 64, wherein L8 is a phosphorothioate internucleoside linkage having the (Rp) configuration and where each of L1, L2, L3, L4, L5, L6, L7 and L9 is a phosphorothioate internucleoside linkage having the (Sp) configuration.

73. The chirally enriched population of oligomeric compounds of claim 64, wherein L9 is a phosphorothioate internucleoside linkage having the (Rp) configuration and where each of L1, L2, L3, L4, L5, L6, L7 and L8 is a phosphorothioate internucleoside linkage having the (Sp) configuration.

74. The chirally enriched population of oligomeric compounds of claim 64, wherein each of L1 and L₂ is a phosphorothioate internucleoside linkage having the (Rp) configuration and where each of L3, L4, L5, L6, L7, L8 and L9 is a phosphorothioate internucleoside linkage having the (Sp) configuration.

75. The chirally enriched population of oligomeric compounds of claim 64, wherein each of L₂ and L3 is a phosphorothioate internucleoside linkage having the (Rp) configuration and where each of L1, L4, L5, L6, L7, L8 and L9 is a phosphorothioate internucleoside linkage having the (Sp) configuration.

76. The chirally enriched population of oligomeric compounds of claim 64, wherein each of L3 and L4 is a phosphorothioate internucleoside linkage having the (Rp) configuration and where each of L1, L2, L5, L6, L7, L8 and L9 is a phosphorothioate internucleoside linkage having the (Sp) configuration.

77. The chirally enriched population of oligomeric compounds of claim 64, wherein each of L4 and L5 is a phosphorothioate internucleoside linkage having the (Rp) configuration and where each of L1, L2, L3, L6, L7, L8 and L9 is a phosphorothioate internucleoside linkage having the (Sp) configuration.

78. The chirally enriched population of oligomeric compounds of claim 64, wherein each of L5 and L6 is a phosphorothioate internucleoside linkage having the (Rp) configuration and where each of L1, L2, L3, L4, L7, L8 and L9 is a phosphorothioate internucleoside linkage having the (Sp) configuration.

79. The chirally enriched population of oligomeric compounds of claim 64, wherein each of L6 and L7 is a phosphorothioate internucleoside linkage having the (Rp) configuration and where each of L1, L2, L3, L4, L5, L8 and L9 is a phosphorothioate internucleoside linkage having the (Sp) configuration.

80. The chirally enriched population of oligomeric compounds of claim 64, wherein each of L7 and L8 is a phosphorothioate internucleoside linkage having the (Rp) configuration and where each of L1, L2, L3, L4, L5, L6 and L9 is a phosphorothioate internucleoside linkage having the (Sp) configuration.

81. The chirally enriched population of oligomeric compounds of claim 64, wherein each of L8 and L9 is a phosphorothioate internucleoside linkage having the (Rp) configuration and where each of L1, L2, L3, L4, L5, L6 and L7 is a phosphorothioate internucleoside linkage having the (Sp) configuration.

82. The chirally enriched population of oligomeric compounds of claim 64, wherein each L1 is a phosphorothioate internucleoside linkage having the (Rp) configuration, each L₂ is a phosphorothioate internucleoside linkage having the (Sp) configuration, and where each of L3, L4, L5, L6, L7, L8 and L9 is a stereorandom phosphorothioate internucleoside linkage.

83. The chirally enriched population of oligomeric compounds of claim 64, wherein each L₂ is a phosphorothioate internucleoside linkage having the (Rp) configuration, each L3 is a phosphorothioate internucleoside linkage having the (Sp) configuration, and where each of L1, L4, L5, L6, L7, L8 and L9 is a stereorandom phosphorothioate internucleoside linkage.

84. The chirally enriched population of oligomeric compounds of claim 64, wherein each L3 is a phosphorothioate internucleoside linkage having the (Rp) configuration, each L4 is a phosphorothioate internucleoside linkage having the (Sp) configuration, and where each of L1, L2, L5, L6, L7, L8 and L9 is a stereorandom phosphorothioate internucleoside linkage.

85. The chirally enriched population of oligomeric compounds of claim 64, wherein each L4 is a phosphorothioate internucleoside linkage having the (Rp) configuration, each L5 is a phosphorothioate internucleoside linkage having the (Sp) configuration, and where each of L1, L2, L3, L6, L7, L8 and L9 is a stereorandom phosphorothioate internucleoside linkage.

86. The chirally enriched population of oligomeric compounds of claim 64, wherein each L5 is a phosphorothioate internucleoside linkage having the (Rp) configuration, each L6 is a phosphorothioate internucleoside linkage having the (Sp) configuration, and where each of L1, L2, L3, L4, L7, L8 and L9 is a stereorandom phosphorothioate internucleoside linkage.

87. The chirally enriched population of oligomeric compounds of claim 64, wherein each L6 is a phosphorothioate internucleoside linkage having the (Rp) configuration, each L7 is a phosphorothioate internucleoside linkage having the (Sp) configuration, and where each of L1, L2, L3, L4, L5, L8 and L9 is a stereorandom phosphorothioate internucleoside linkage.

88. The chirally enriched population of oligomeric compounds of claim 64, wherein each L7 is a phosphorothioate internucleoside linkage having the (Rp) configuration, each L8 is a phosphorothioate internucleoside linkage having the (Sp) configuration, and where each of L1, L2, L3, L4, L5, L6 and L9 is a stereorandom phosphorothioate internucleoside linkage.

89. The chirally enriched population of oligomeric compounds of claim 64, wherein each L8 is a phosphorothioate internucleoside linkage having the (Rp) configuration, each L9 is a phosphorothioate internucleoside linkage having the (Sp) configuration, and where each of L1, L2, L3, L4, L5, L6 and L7 is a stereorandom phosphorothioate internucleoside linkage.

90. The oligomeric compound of claim 1 or 2, wherein the central region consists of 10 linked nucleosides and has the formula:
(N_{d})_{L1}(N_{d})_{L2}(N_{d})_{L3}(N_{d})_{L4}(N_{d})_{L5}(N_{d})_{L6}(N_{d})_{L7}(N_{d})_{L8}(N_{d})_{L9}(N_{d})_{L10}; wherein
each N_{d} is a 2′-β-D-deoxyribosyl sugar moiety and each of L1, L2, L3, L4, L5, L6, L7, L8, L9, and L10 is a phosphorothioate internucleoside linkage; and
wherein at least one of L1, L2, L3, L4, L5, L6, L7, L8, L9 and L10 has the (Sp) configuration or the (Rp) configuration.

91. The chirally enriched population of oligomeric compounds of claim 69 or 90, wherein L1 is a phosphorothioate internucleoside linkage having the (Rp) configuration and where each of L2, L3, L4, L5, L6, L7, L8, L9 and L10 is a phosphorothioate internucleoside linkage having the (Sp) configuration.

92. The chirally enriched population of oligomeric compounds of claim 90, wherein L₂ is a phosphorothioate internucleoside linkage having the (Rp) configuration and where each of L1, L3, L4, L5, L6, L7, L8, L9 and L10 is a phosphorothioate internucleoside linkage having the (Sp) configuration.

93. The chirally enriched population of oligomeric compounds of claim 90, wherein L3 is a phosphorothioate internucleoside linkage having the (Rp) configuration and where each of L1, L2, L4, L5, L6, L7, L8, L9 and L10 is a phosphorothioate internucleoside linkage having the (Sp) configuration.

94. The chirally enriched population of oligomeric compounds of claim 90, wherein L4 is a phosphorothioate internucleoside linkage having the (Rp) configuration and where each of L1, L2, L3, L5, L6, L7, L8, L9 and L10 is a phosphorothioate internucleoside linkage having the (Sp) configuration.

95. The chirally enriched population of oligomeric compounds of claim 90, wherein L5 is a phosphorothioate internucleoside linkage having the (Rp) configuration and where each of L1, L2, L3, L4, L6, L7, L8, L9 and L10 is a phosphorothioate internucleoside linkage having the (Sp) configuration.

96. The chirally enriched population of oligomeric compounds of claim 90, wherein L6 is a phosphorothioate internucleoside linkage having the (Rp) configuration and where each of L1, L2, L3, L4, L5, L7, L8, L9 and L10 is a phosphorothioate internucleoside linkage having the (Sp) configuration.

97. The chirally enriched population of oligomeric compounds of claim 90, wherein L7 is a phosphorothioate internucleoside linkage having the (Rp) configuration and where each of L1, L2, L3, L4, L5, L6, L8, L9 and L10 is a phosphorothioate internucleoside linkage having the (Sp) configuration.

98. The chirally enriched population of oligomeric compounds of claim 90, wherein L8 is a phosphorothioate internucleoside linkage having the (Rp) configuration and where each of L1, L2, L3, L4, L5, L6, L7, L9 and L10 is a phosphorothioate internucleoside linkage having the (Sp) configuration.

99. The chirally enriched population of oligomeric compounds of claim 90, wherein L9 is a phosphorothioate internucleoside linkage having the (Rp) configuration and where each of L1, L2, L3, L4, L5, L6, L7, L8 and L10 is a phosphorothioate internucleoside linkage having the (Sp) configuration.

100. The chirally enriched population of oligomeric compounds of claim 90, wherein L10 is a phosphorothioate internucleoside linkage having the (Rp) configuration and where each of L1, L2, L3, L4, L5, L6, L7, L8 and L9 is a phosphorothioate internucleoside linkage having the (Sp) configuration.

101. The chirally enriched population of oligomeric compounds of claim 90, wherein each of L1 and L₂ is a phosphorothioate internucleoside linkage having the (Rp) configuration and where each of L3, L4, L5, L6, L7, L8, L9 and L10 is a phosphorothioate internucleoside linkage having the (Sp) configuration.

102. The chirally enriched population of oligomeric compounds of claim 90, wherein each of L₂ and L3 is a phosphorothioate internucleoside linkage having the (Rp) configuration and where each of L1, L4, L5, L6, L7, L8, L9 and L10 is a phosphorothioate internucleoside linkage having the (Sp) configuration.

103. The chirally enriched population of oligomeric compounds of claim 90, wherein each of L3 and L4 is a phosphorothioate internucleoside linkage having the (Rp) configuration and where each of L1, L2, L5, L6, L7, L8, L9 and L10 is a phosphorothioate internucleoside linkage having the (Sp) configuration.

104. The chirally enriched population of oligomeric compounds of claim 90, wherein each of L4 and L5 is a phosphorothioate internucleoside linkage having the (Rp) configuration and where each of L1, L2, L3, L6, L7, L8, L9 and L10 is a phosphorothioate internucleoside linkage having the (Sp) configuration.

105. The chirally enriched population of oligomeric compounds of claim 90, wherein each of L5 and L6 is a phosphorothioate internucleoside linkage having the (Rp) configuration and where each of L1, L2, L3, L4, L7, L8, L9 and L10 is a phosphorothioate internucleoside linkage having the (Sp) configuration.

106. The chirally enriched population of oligomeric compounds of claim 90, wherein each of L6 and L7 is a phosphorothioate internucleoside linkage having the (Rp) configuration and where each of L1, L2, L3, L4, L5, L8, L9 and L10 is a phosphorothioate internucleoside linkage having the (Sp) configuration.

107. The chirally enriched population of oligomeric compounds of claim 90, wherein each of L7 and L8 is a phosphorothioate internucleoside linkage having the (Rp) configuration and where each of L1, L2, L3, L4, L5, L6, L9 and L10 is a phosphorothioate internucleoside linkage having the (Sp) configuration.

108. The chirally enriched population of oligomeric compounds of claim 90, wherein each of L8 and L9 is a phosphorothioate internucleoside linkage having the (Rp) configuration and where each of L1, L2, L3, L4, L5, L6, L7 and L10 is a phosphorothioate internucleoside linkage having the (Sp) configuration.

109. The chirally enriched population of oligomeric compounds of claim 90, wherein each of L9 and L10 is a phosphorothioate internucleoside linkage having the (Rp) configuration and where each of L1, L2, L3, L4, L5, L6, L7 and L8 is a phosphorothioate internucleoside linkage having the (Sp) configuration.

110. The chirally enriched population of oligomeric compounds of claim 90, wherein each of L3, L6, L9 and L10 is a phosphorothioate internucleoside linkage having the (Rp) configuration and where each of L1, L2, L4, L5, L7, and L8 is a phosphorothioate internucleoside linkage having the (Sp) configuration.

111. The chirally enriched population of oligomeric compounds of claim 90, wherein each L1 is a phosphorothioate internucleoside linkage having the (Rp) configuration, each L₂ is a phosphorothioate internucleoside linkage having the (Sp) configuration, and where each of L3, L4, L5, L6, L7, L8, L9 and L10 is a stereorandom phosphorothioate internucleoside linkage.

112. The chirally enriched population of oligomeric compounds of claim 90, wherein each L₂ is a phosphorothioate internucleoside linkage having the (Rp) configuration, each L3 is a phosphorothioate internucleoside linkage having the (Sp) configuration, and where each of L1, L4, L5, L6, L7, L8, L9 and L10 is a stereorandom phosphorothioate internucleoside linkage.

113. The chirally enriched population of oligomeric compounds of claim 90, wherein each L3 is a phosphorothioate internucleoside linkage having the (Rp) configuration, each L4 is a phosphorothioate internucleoside linkage having the (Sp) configuration, and where each of L1, L2, L5, L6, L7, L8, L9 and L10 is a stereorandom phosphorothioate internucleoside linkage.

114. The chirally enriched population of oligomeric compounds of claim 90, wherein each L4 is a phosphorothioate internucleoside linkage having the (Rp) configuration, each L5 is a phosphorothioate internucleoside linkage having the (Sp) configuration, and where each of L1, L2, L3, L6, L7, L8, L9 and L10 is a stereorandom phosphorothioate internucleoside linkage.

115. The chirally enriched population of oligomeric compounds of claim 90, wherein each L5 is a phosphorothioate internucleoside linkage having the (Rp) configuration, each L6 is a phosphorothioate internucleoside linkage having the (Sp) configuration, and where each of L1, L2, L3, L4, L7, L8, L9 and L10 is a stereorandom phosphorothioate internucleoside linkage.

116. The chirally enriched population of oligomeric compounds of claim 90, wherein each L6 is a phosphorothioate internucleoside linkage having the (Rp) configuration, each L7 is a phosphorothioate internucleoside linkage having the (Sp) configuration, and where each of L1, L2, L3, L4, L5, L8, L9 and L10 is a stereorandom phosphorothioate internucleoside linkage.

117. The chirally enriched population of oligomeric compounds of claim 90, wherein each L7 is a phosphorothioate internucleoside linkage having the (Rp) configuration, each L8 is a phosphorothioate internucleoside linkage having the (Sp) configuration, and where each of L1, L2, L3, L4, L5, L6, L9 and L10 is a stereorandom phosphorothioate internucleoside linkage.

118. The chirally enriched population of oligomeric compounds of claim 90, wherein each L8 is a phosphorothioate internucleoside linkage having the (Rp) configuration, each L9 is a phosphorothioate internucleoside linkage having the (Sp) configuration, and where each of L1, L2, L3, L4, L5, L6, L7 and L10 is a stereorandom phosphorothioate internucleoside linkage.

119. The chirally enriched population of oligomeric compounds of claim 90, wherein each L9 is a phosphorothioate internucleoside linkage having the (Rp) configuration, each L10 is a phosphorothioate internucleoside linkage having the (Sp) configuration, and where each of L1, L2, L3, L4, L5, L6, L7 and L8 is a stereorandom phosphorothioate internucleoside linkage.

120. The oligomeric compound of claim 1 or 2, wherein the central region consists of 11 linked nucleosides and has the formula:
(Nd)L1(Nd)L2(Nd)L3(Nd)L4(Nd)L5(Nd)L6(Nd)L7(Nd)L8(Nd)L9(Nd)L10(Nd)L11;
wherein,
each N_{d} is a 2′-β-D-deoxyribosyl sugar moiety and each of L1, L2, L3, L4, L5, L6, L7, L8, L9, L10 and L11 is a phosphorothioate internucleoside linkage; and
wherein at least one of L1, L2, L3, L4, L5, L6, L7, L8, L9, L10 and L11 has the (Sp) configuration or the (Rp) configuration.

121. The chirally enriched population of oligomeric compounds of claim 120, wherein L1 is a phosphorothioate internucleoside linkage having the (Rp) configuration and where each of L2, L3, L4, L5, L6, L7, L8, L9, L10 and L11 is a phosphorothioate internucleoside linkage having the (Sp) configuration.

122. The chirally enriched population of oligomeric compounds of claim 120, wherein L₂ is a phosphorothioate internucleoside linkage having the (Rp) configuration and where each of L1, L3, L4, L5, L6, L7, L8, L9, L10 and L11 is a phosphorothioate internucleoside linkage having the (Sp) configuration.

123. The chirally enriched population of oligomeric compounds of claim 120, wherein L3 is a phosphorothioate internucleoside linkage having the (Rp) configuration and where each of L1, L2, L4, L5, L6, L7, L8, L9, L10 and L11 is a phosphorothioate internucleoside linkage having the (Sp) configuration.

124. The chirally enriched population of oligomeric compounds of claim 120, wherein L4 is a phosphorothioate internucleoside linkage having the (Rp) configuration and where each of L1, L2, L3, L5, L6, L7, L8, L9, L10 and L11 is a phosphorothioate internucleoside linkage having the (Sp) configuration.

125. The chirally enriched population of oligomeric compounds of claim 120, wherein L5 is a phosphorothioate internucleoside linkage having the (Rp) configuration and where each of L1, L2, L3, L4, L6, L7, L8, L9, L10 and L11 is a phosphorothioate internucleoside linkage having the (Sp) configuration.

126. The chirally enriched population of oligomeric compounds of claim 120, wherein L6 is a phosphorothioate internucleoside linkage having the (Rp) configuration and where each of L1, L2, L3, L4, L5, L7, L8, L9, L10 and L11 is a phosphorothioate internucleoside linkage having the (Sp) configuration.

127. The chirally enriched population of oligomeric compounds of claim 120, wherein L7 is a phosphorothioate internucleoside linkage having the (Rp) configuration and where each of L1, L2, L3, L4, L5, L6, L8, L9, L10 and L11 is a phosphorothioate internucleoside linkage having the (Sp) configuration.

128. The chirally enriched population of oligomeric compounds of claim 120, wherein L8 is a phosphorothioate internucleoside linkage having the (Rp) configuration and where each of L1, L2, L3, L4, L5, L6, L7, L9, L10 and L11 is a phosphorothioate internucleoside linkage having the (Sp) configuration.

129. The chirally enriched population of oligomeric compounds of claim 120, wherein L9 is a phosphorothioate internucleoside linkage having the (Rp) configuration and where each of L1, L2, L3, L4, L5, L6, L7, L8, L10 and L11 is a phosphorothioate internucleoside linkage having the (Sp) configuration.

130. The chirally enriched population of oligomeric compounds of claim 120, wherein L10 is a phosphorothioate internucleoside linkage having the (Rp) configuration and where each of L1, L2, L3, L4, L5, L6, L7, L8, L9 and L11 is a phosphorothioate internucleoside linkage having the (Sp) configuration.

131. The chirally enriched population of oligomeric compounds of claim 120, wherein L11 is a phosphorothioate internucleoside linkage having the (Rp) configuration and where each of L1, L2, L3, L4, L5, L6, L7, L8, L9 and L10 is a phosphorothioate internucleoside linkage having the (Sp) configuration.

132. The chirally enriched population of oligomeric compounds of claim 120, wherein each of L1 and L₂ is a phosphorothioate internucleoside linkage having the (Rp) configuration and where each of L3, L4, L5, L6, L7, L8, L9, L10 and L11 is a phosphorothioate internucleoside linkage having the (Sp) configuration.

133. The chirally enriched population of oligomeric compounds of claim 120, wherein each of L₂ and L3 is a phosphorothioate internucleoside linkage having the (Rp) configuration and where each of L1, L4, L5, L6, L7, L8, L9, L10 and L11 is a phosphorothioate internucleoside linkage having the (Sp) configuration.

134. The chirally enriched population of oligomeric compounds of claim 120, wherein each of L₂ and L3 is a phosphorothioate internucleoside linkage having the (Rp) configuration and where each of L1, L4, L5, L6, L7, L8, L9, L10 and L11 is a phosphorothioate internucleoside linkage having the (Sp) configuration.

135. The chirally enriched population of oligomeric compounds of claim 120, wherein each of L3 and L4 is a phosphorothioate internucleoside linkage having the (Rp) configuration and where each of L1, L2, L5, L6, L7, L8, L9, L10 and L11 is a phosphorothioate internucleoside linkage having the (Sp) configuration.

136. The chirally enriched population of oligomeric compounds of claim 120, wherein each of L4 and L5 is a phosphorothioate internucleoside linkage having the (Rp) configuration and where each of L1, L2, L3, L6, L7, L8, L9, L10 and L11 is a phosphorothioate internucleoside linkage having the (Sp) configuration.

137. The chirally enriched population of oligomeric compounds of claim 120, wherein each of L5 and L6 is a phosphorothioate internucleoside linkage having the (Rp) configuration and where each of L1, L2, L3, L4, L7, L8, L9, L10 and L11 is a phosphorothioate internucleoside linkage having the (Sp) configuration.

138. The chirally enriched population of oligomeric compounds of claim 120, wherein each of L6 and L7 is a phosphorothioate internucleoside linkage having the (Rp) configuration and where each of L1, L2, L3, L4, L5, L8, L9, L10 and L11 is a phosphorothioate internucleoside linkage having the (Sp) configuration.

139. The chirally enriched population of oligomeric compounds of claim 120, wherein each of L7 and L8 is a phosphorothioate internucleoside linkage having the (Rp) configuration and where each of L1, L2, L3, L4, L5, L6, L9, L10 and L11 is a phosphorothioate internucleoside linkage having the (Sp) configuration.

140. The chirally enriched population of oligomeric compounds of claim 120, wherein each of L8 and L9 is a phosphorothioate internucleoside linkage having the (Rp) configuration and where each of L1, L2, L3, L4, L5, L6, L7, L10 and L11 is a phosphorothioate internucleoside linkage having the (Sp) configuration.

141. The chirally enriched population of oligomeric compounds of claim 120, wherein each of L9 and L10 is a phosphorothioate internucleoside linkage having the (Rp) configuration and where each of L1, L2, L3, L4, L5, L6, L7, L9 and L11 is a phosphorothioate internucleoside linkage having the (Sp) configuration.

142. The chirally enriched population of oligomeric compounds of claim 120, wherein each of L10 and L11 is a phosphorothioate internucleoside linkage having the (Rp) configuration and where each of L1, L2, L3, L4, L5, L6, L7, L8 and L9 is a phosphorothioate internucleoside linkage having the (Sp) configuration.

143. The chirally enriched population of oligomeric compounds of claim 120, wherein each L1 is a phosphorothioate internucleoside linkage having the (Rp) configuration, each L₂ is a phosphorothioate internucleoside linkage having the (Sp) configuration, and where each of L3, L4, L5, L6, L7, L8, L9, L10 and L11 is a stereorandom phosphorothioate internucleoside linkage.

144. The chirally enriched population of oligomeric compounds of claim 120, wherein each L₂ is a phosphorothioate internucleoside linkage having the (Rp) configuration, each L3 is a phosphorothioate internucleoside linkage having the (Sp) configuration, and where each of L1, L4, L5, L6, L7, L8, L9, L10 and L11 is a stereorandom phosphorothioate internucleoside linkage.

145. The chirally enriched population of oligomeric compounds of claim 120, wherein each L3 is a phosphorothioate internucleoside linkage having the (Rp) configuration, each L4 is a phosphorothioate internucleoside linkage having the (Sp) configuration, and where each of L1, L2, L5, L6, L7, L8, L9, L10 and L11 is a stereorandom phosphorothioate internucleoside linkage.

146. The chirally enriched population of oligomeric compounds of claim 120, wherein each L4 is a phosphorothioate internucleoside linkage having the (Rp) configuration, each L5 is a phosphorothioate internucleoside linkage having the (Sp) configuration, and where each of L1, L2, L3, L6, L7, L8, L9, L10 and L11 is a stereorandom phosphorothioate internucleoside linkage.

147. The chirally enriched population of oligomeric compounds of claim 120, wherein each L5 is a phosphorothioate internucleoside linkage having the (Rp) configuration, each L6 is a phosphorothioate internucleoside linkage having the (Sp) configuration, and wherein each of L1, L2, L3, L4, L7, L8, L9, L10 and L11 is a stereorandom phosphorothioate internucleoside linkage.

148. The chirally enriched population of oligomeric compounds of claim 120, wherein each L6 is a phosphorothioate internucleoside linkage having the (Rp) configuration, each L7 is a phosphorothioate internucleoside linkage having the (Sp) configuration, and where each of L1, L2, L3, L4, L5, L8, L9, L10 and L11 is a stereorandom phosphorothioate internucleoside linkage.

149. The chirally enriched population of oligomeric compounds of claim 120, wherein each L7 is a phosphorothioate internucleoside linkage having the (Rp) configuration, each L8 is a phosphorothioate internucleoside linkage having the (Sp) configuration, and where each of L1, L2, L3, L4, L5, L6, L9, L10 and L11 is a stereorandom phosphorothioate internucleoside linkage.

150. The chirally enriched population of oligomeric compounds of claim 120, wherein each L8 is a phosphorothioate internucleoside linkage having the (Rp) configuration, each L9 is a phosphorothioate internucleoside linkage having the (Sp) configuration, and where each of L1, L2, L3, L4, L5, L6, L7, L10 and L11 is a stereorandom phosphorothioate internucleoside linkage.

151. The chirally enriched population of oligomeric compounds of claim 120, wherein each L9 is a phosphorothioate internucleoside linkage having the (Rp) configuration, each L10 is a phosphorothioate internucleoside linkage having the (Sp) configuration, and where each of L1, L2, L3, L4, L5, L6, L7, L8 and L11 is a stereorandom phosphorothioate internucleoside linkage.

152. The chirally enriched population of oligomeric compounds of claim 120, wherein each L10 is a phosphorothioate internucleoside linkage having the (Rp) configuration, each L11 is a phosphorothioate internucleoside linkage having the (Sp) configuration, and where each of L1, L2, L3, L4, L5, L6, L7, L8 and L9 is a stereorandom phosphorothioate internucleoside linkage.

153. The chirally enriched population of oligomeric compounds of any of claims 1-152, wherein the 5′-region consists of 2-4 linked nucleosides.

154. The chirally enriched population of oligomeric compounds of any of claims 1-152, wherein the 5′-region consists of 1 nucleoside.

155. The chirally enriched population of oligomeric compounds of any of claims 1-152, wherein the 5′-region consists of 2 linked nucleosides.

156. The chirally enriched population of oligomeric compounds of any of claims 1-152, wherein the 5′-region consists of 3 linked nucleosides.

157. The chirally enriched population of oligomeric compounds of any of claims 1-152, wherein the 5′-region consists of 4 linked nucleosides.

158. The chirally enriched population of oligomeric compounds of any of claims 1-152, wherein the 5′-region consists of 5 linked nucleosides.

159. The chirally enriched population of oligomeric compounds of any of claims 1-158, wherein each nucleoside of the 5′-region comprises a 2′-modified ribosyl sugar moiety.

160. The chirally enriched population of oligomeric compounds of any of claims 1-159, wherein at least one nucleoside of the 5′-region comprises a bicyclic furanosyl sugar moiety.

161. The chirally enriched population of oligomeric compounds of any of claims 1-160, wherein each nucleoside of the 5′-region comprises a bicyclic furanosyl sugar moiety.

162. The chirally enriched population of oligomeric compounds of any of claims 1-160 wherein at least one nucleoside of the 5′-region comprises a non-bicyclic sugar moiety.

163. The chirally enriched population of oligomeric compounds of claim 162, wherein the non-bicyclic sugar moiety of the at least one nucleoside of the 5′-region is a 2′-substituted ribosyl sugar moiety.

164. The chirally enriched population of oligomeric compounds of any of claim 1-159 or 162-163, wherein each nucleoside of the 5′-region comprises a non-bicyclic sugar moiety.

165. The chirally enriched population of oligomeric compounds of claim 164, wherein each nucleoside of the 5′-region comprises a 2′-substituted ribosyl sugar moiety.

166. The chirally enriched population of oligomeric compounds of any of claims 1-159, wherein each nucleoside of the 5′-region comprises a 2′-modified furanosyl sugar moiety independently selected from a bicyclic sugar moiety and a non-bicyclic, 2′-substituted ribosyl sugar moiety.

167. The chirally enriched population of oligomeric compounds of any of claim 160-163 or 166, wherein each bicyclic sugar moiety of the 5′-region is selected from among cEt, LNA, and ENA.

168. The chirally enriched population of oligomeric compounds of any of claims 162-166, wherein each non-bicyclic sugar moiety of the 5′-region has a 2′-substituent selected from among 2′-MOE, 2′-OMe, and 2′-NMA.

169. The chirally enriched population of oligomeric compounds of any of claims 1-168, wherein none of the nucleosides of the 5′-region comprise a sugar moiety having a 2′-F substituent.

170. The chirally enriched population of oligomeric compounds of any of claims 1-169, wherein each nucleobase of the 5′-region is independently selected from among thymine, uracil, guanine, cytosine, 5-methylcytosine, and adenine.

171. The chirally enriched population of oligomeric compounds of any of claims 1-170, wherein each internucleoside linkage of the 5′-region is selected from among phosphodiester and phosphorothioate internucleoside linkages.

172. The chirally enriched population of oligomeric compounds of any of claims 1-171, wherein the 3′-region consists of 2-4 linked nucleosides.

173. The chirally enriched population of oligomeric compounds of any of claims 1-171, wherein the 3′-region consists of 1 nucleoside.

174. The chirally enriched population of oligomeric compounds of any of claims 1-171, wherein the 3′-region consists of 2 linked nucleosides.

175. The chirally enriched population of oligomeric compounds of any of claims 1-171, wherein the 3′-region consists of 3 linked nucleosides.

176. The chirally enriched population of oligomeric compounds of any of claims 1-170, wherein the 3′-region consists of 4 linked nucleosides.

177. The chirally enriched population of oligomeric compounds of any of claims 1-171, wherein the 3′-region consists of 5 linked nucleosides.

178. The chirally enriched population of oligomeric compounds of any of claims 1-177, wherein each nucleoside of the 3′-region comprises a 2′-modified ribosyl sugar moiety.

179. The chirally enriched population of oligomeric compounds of any of claims 1-178, wherein at least one nucleoside of the 3′-region comprises a bicyclic furanosyl sugar moiety.

180. The chirally enriched population of oligomeric compounds of any of claims 1-179, wherein each nucleoside of the 3′-region comprises a bicyclic furanosyl sugar moiety.

181. The chirally enriched population of oligomeric compounds of any of claims 1-179, wherein at least one nucleoside of the 3′-region comprises a non-bicyclic sugar moiety.

182. The chirally enriched population of oligomeric compounds of claim 1-181, wherein the non-bicyclic sugar moiety of the at least one nucleoside of the 3′-region is a 2′-substituted ribosyl sugar moiety.

183. The chirally enriched population of oligomeric compounds of any of claim 1-178 or 181-182, wherein each nucleoside of the 3′-region comprises a non-bicyclic sugar moiety.

184. The chirally enriched population of oligomeric compounds of claim 183, wherein each nucleoside of the 3′-region comprises a 2′-substituted ribosyl sugar moiety.

185. The chirally enriched population of oligomeric compounds of any of claims 1-178, wherein each nucleoside of the 3′-region comprises a 2′-modified furanosyl sugar moiety independently selected from a bicyclic sugar moiety and a non-bicyclic, 2′-substituted ribosyl sugar moiety.

186. The chirally enriched population of oligomeric compounds of any of claim 179-182 or 185, wherein each bicyclic sugar moiety of the 3′-region is selected from among cEt, LNA, and ENA.

187. The chirally enriched population of oligomeric compounds of any of claims 181-185, wherein each non-bicyclic sugar moiety of the 3′-region has a 2′-substituent selected from among 2′-MOE, 2′-OMe, and 2′-NMA.

188. The chirally enriched population of oligomeric compounds of any of claims 1-187, wherein none of the nucleosides of the 3′-region comprise a sugar moiety having a 2′-F substituent.

189. A pharmaceutical composition comprising, the chirally enriched population of oligomeric compounds of any of claims 1-187.

190. A method comprising, contacting a cell with the chirally enriched population of oligomeric compounds of any of claims 1-187.

191. A method of modulating the amount or activity of a target nucleic acid in a cell, comprising contacting a cell with the chirally enriched population of compounds of any of claims 1-187.


SEQUENCE LISTING (5 sequences)

SEQ ID NO: 1 - DNA, 14836 bp, Mus musculus
cctcccccgtgtctccccacacccgggttggggttgttttggttgaccagagtggaacacaacgatctattggcagggctgaacaccaatgggtctatttgtaaagcgccaatgaccactttctgaagcagggttttagggagcggggccttagggaactctttggtcctttttagaacactggactttcttctggaaaggcaggaaacactgaagtttaagaagttgtttccagcttccattaactgaacacacattaaaaccaagcacagagaatcaggacgtttcgcgggagtgagacccagtcatttctcctccgtttccattctgcagggtgagagttgtaatcacccacccactattcgtaccatccacccacccccagtcgagagaataggggtacagaggggaggtggcaaagaaaattcacgatactgagtatctctgggagacctgtttggtctctttgctcggtagcgcagccctacgttagaatgcatcttcccgggaatgactgtagtgagactttggctgggaatccaagttattctaactgtagattggtccacgttgccctaagcctagcagtccactgcggcacagacaccctggacatgaggtgggtcagcttaagttcctggcacgaaagaaagggtactctggcaacttttggatgcggcgaaacagactgtttcgtctctcaggttcttatttcacggcttgtgcctttgacagccccttagtttctctatctgcaggatgggagcattaagctctacgacccagcctctttacaattcaggtccaaagagcccgcccaagttggggactgggaagatcaaaggtctcagcacccagcggagccgcggacactgagggcgccaagaagggggtgggtaggtagggaactggaagggcggctgctccgcaggggatgcgcgtcagagaccccagccacactccaggcccgccccttgatgagccccgccccgccccgcctggttttcgcctctaaagcgcccagcgctcgcctcccgctgccgcactttcactctcggtccacctcggtgtcctcttgctgtccagctctgcagcctccggcgcgccctcccgcccacgccatggacgccaaggtcgtcgccgtgctggccctggtgctggccgcgctctgcatcagtgacggtgagtgcaatccgcggccgggcccgggaaaggctcgcagctctgcgccggagctccttcgggtccgcggttcctctgcccgcgccgaagtcgcggagaaagaactcggtcggcgccgttcactacaagcgaacttggggcagtccactttgcagggcgcactcccaccgggtgccctttcccgtgtcccacgggtcgcaccgaggttttgtgctctgcgaagtgcggccataggacctagagagggctgcaggggaggacccgcaggattgttgggcaagagtgggttcggcgcggaatggaagcgtgggcgattgtgtccggggcttgggccccggagcgcgccagctgcactcagctagtgtctaccggcgcccagatgtttccagaggcgaagggcagcgcggtcccggagttgaccgtgcaagaggttcactcgggtggtgcgtgtgtcagcaaactctcaaagaccggtcaagtagctcgaagtgcatggcttggctataggttcagtggtgaggctgagtttcgtcccctgcgggtgtagcgtgttctcttacagcaccctcgaggggctcagggccaccagcagcgcagcgcagctcttgaactcgcgctgccagccagggccgcgcttctgcacagttcgttggtccgtagcgacgcggacctgagcacgcgtctcttcactgcccctttttcttcttacccgggtcactagacaaaggctcagcagttacccaagctatatgcacacctctccccaacccccaaacacacctgcaaacgggcgcttttgtagccagccccggagtcctcagctctggaatgagagctgcagcggagttcagtctcccagacccagggtggtgtcttctttcactgggaaagggctttcattttgttttctttttttgacactgaagagaaaactctcagcgctgttacaagacaccgttgctgcaaaacaaaacaaaccattgcctctgaacacaaaacaaaatcctactagtcgatcccctgccttcctccgcagtggtgtttcctggagagaactgagggacagtcggggctcttggtgagactgagctctaaatgctgcccaagtacaccaactcgttcgtttgggttctttccctgtgacaacggggtacgggaatggttggagttgcctagtccgagggaaatgttctgtaaaagaatagtcagttgctgatcggagtagtaaaaaaaaagaaatgaaaggcagtttcgatttttttttttttttttttttttttttttgttaccgagaacacccgggaggctgagccttcccactggtcccccagtgccccgtcatggagcacattgatttgggcattaataattgaatgagctggtgatgttgcaagggtcacagcctctggcaagttaggtatggggcaagaatgtaggactcaggtcctcaaggttggagtgcaattatccagagtaaaagttgtctcaccctcaacatattctgaccctaggaagagtcggattgttgacagtgtctggatcagacctgttctctaggcaggaccccattgtgctgcccgaatgaacttttttacctcctagtgcctgtgtgccctctgatcttacacagccctcaagttgcagcacggctaaccttgctgtggttcctgtcttttcccatcagctactccaactcagaagctagatagtagacaccggaggcttctttggttaaacccagagcagcaggcttgccaggcttgttagattgaatggacccctggttccctaagccaagctctctagattcccaagtccagggtggcagcagagctggattagactttggtctgtacctgaagtctggttttcctatgctttagagtctaaagacactacccttcctggggcatgcatcccttagctaaataatgcttgcagaagaagataatcccatcatatatttaattcggtccacttctccagctgcttcccaaaggcagtgaacttcagaatacccagaagtctcctggaactctaaataagcaaacttaaaatcctggggctaactattctcagtcatacttttaaactttggtgaaaagacccataaattgaaacatttggggatgctcagtagagctaggataaaaccctgttgttgggggagcagctacaaatccagcagtcctcagggtttgcaattctagacttaaagggtggttcttaagggggggttctaaaggagccccttgctaatttacactaatgagtgtcaattatagcattttgcaaattggtgaattggcaaacaaagctggtaataggatccaggaggcctaggcatccaggtagtgaccataaaagccacggttgaccccagcttttgggaaaagctggatagaaggtaaatccgggtcctcccctctggattcttttgtgatttccagggcttaggatagggtgagtgggaggagggaaaactgcaggtggtagaagtgaagccccccacctccaggcctgcaccagagggccacaagggagcccagaactctgccaccccacttctcctgggtccttttgtcctttagaggctgagcccagtcagatctcactgtgatccctggccgaggggatggtctttgcaagaaactttctgtaaccattcctgctgatgttcctgagtcttccccacaagagccaccaaaccccctgcaccaggcagataatgactggccccacttttctctctacacctcctctaggtaaaccagtcagcctgagctaccgatgcccctgccggttcttcgagagccacatcgccagagccaacgtcaagcatctgaaaatcctcaacactccaaactgtgcccttcagattgtgtaagtcctagccgccatcccccaaagaggagcatggtatagaagcctcggacttggcataactaggggcagctgttaccaccaccaccacggggacactgatatgccatcagacatgggtttcaaaggatacttttgttccccagagccctgatgtcctcagtgtttctcactcttgctttccaagctgtttcttgcagcacagtgggccgcctctctacagaaaaagccatggacttgatggaggtcagccctcagctgacagttgggtctgtcttgtcagtttcaaggttctggtgtccaaagttaatcctttctcacatagaaaaaaaaattacaagacccggatggcacggggggggggggggttcagttttactcacttgcactcacttgctcagaggtcatttttgttttagagttttagagtttgctggagtgtgatggtagctgccagtatttgatttaaatttacctgggaaataagaaaagcccaaaaaaggtataaatgatgtgaatatctcactcagagtctggtagacttggcagagatgtgtcctgtgctagtctgtcctgctcactgccccccagcaggggttcccatcctcgggagactcaacactaacaacagtataaggatgcagcagctggagcaatgctagcctgacggctttgtcacccaacggtgactgcttcagactttctgtgctcatcagccttcctctccagcctccgctgctgtgttatgtacagtaggctttagagacctagatgatgaatattatttttgctgttttgattaaaatacaatactctcccgagaaagggattttaaagatgatgagtttacgtttgaataggctgtgctggtgcactgtcccgggaagggcccttgaacttagagggtcaaatacaactattgattctgggtgatcactaagttaataaatggcaggatccagactgacacccctgatccctgttgaagttacatccctctgaacgactggtcaactgcagggcagcctgcttgaagagggttacctgtccctaggacactgaacaggcatttgtttttcctagaagacagttcaccagctggagaggagtcgtctcccgtagtttctgtttggttgcttttggtttttgtttggttttggttttttaattatctggcatccaggacttgatggaaaataaccagagctaagctcaccggttcatctgcccattaggaagttctagggatgggagaaagaacacggcgtcaattaacaaatccacaaagctaagaccttgaagcattctgtgaacttgtaaacgcgctcaggcaaccattggacaatttgtctagactgctccttgcccacctgaactgccctgttcctccccttctggactcctgccgtcttcctccagagctacctttaaggttgtcccatgtactatcaaggtgctctgtcaaaagttcttaggctgcttctggcactctccagaattttccaagacctcccccccaccatgatatcagtcatccgcgccttctgggtggttcttcctccacaccctttgggcactttgactcctgtgggatattcgtccttccttttcctttagctttcctcacttgccaagctccaacttggccagaagctcaaatgcctccactgtggtctcttctctgtgtcccctgggagacatccttagcacgtccctaactctgcggtggtggtcccaacacgattcaagtgctatgtcttccaaaactgaagcttccgggagcagcagctgggccctgcagtgaggacctttagctgggtgtgttgggtgagcccacaggatcgctttctcccgcttggctgtacagcgtctctccccttgtgttttggcagtgcacggctgaagaacaacaacagacaagtgtgcattgacccgaaattaaagtggatccaagagtacctggagaaagctttaaacaagtaagcacaacagcccaaaggactttccagtagacccccgaggaaggctgacatccgtgggagatgcaagggcagtggtggggaggagggcctgaaccctggccaggatggccggcgggacagcactgactggggtcatgctaaggtttgccagcataaagacactccgccatagcatatggtacgatattgcagcttatattcatccctgccctcgcccgtgcacaatggagcttttataactggggtttttctaaggaattgtattaccctaaccagttagcttcatccccattctcctcatcctcatcttcattttaaaaagcagtgattacttcaagggctgtattcagtttgctttggagcttctctttgccctggggcctctgggcacagttatagacggtggctttgcagggagccctagagagaaaccttccaccagagcagagtccgaggaacgctgcagggcttgtcctgcagggggcgctcctcgacagatgccttgtcctgagtcaacacaagatccggcagagggaggctcctttatccagttcagtgccagggtcgggaagcttcctttagaagtgatccctgaagctgtgctcagagaccctttcctagccgttcctgctctctgcttgcctccaaacgcatgcttcatctgacttccgcttctcacctctgtagcctgacggaccaatgctgcaatggaagggaggagagtgatgtggggtgccccctccctctcttccctttgctttcctctcacttgggccctttgtgagatttttctttggcctcctgtagaatggagccagaccatcctggataatgtgagaacatgcctagatttacccacaaaacacaagtctgagaattaatcataaacggaagtttaaatgaggatttggactttggtaattgtccctgagtcctatatatttcaacagtggctctatgggctctgatcgaatatcagtgatgaaaataataataataataataataacgaataagccagaatcttgccatgaagccacagtggggattctgggttccaatcagaaatggagacaagataaaacttgcatacattcttatgatcacagacggccctggtggtttttggtaactatttacaaggcatttttttacatatatttttgtgcactttttatgtttctttggaagacaaatgtatttcagaatatatttgtagtcaattcatatatttgaagtggagccatagtaatgccagtagatatctctatgatcttgagctactggcaacttgtaaagaaatatatatgacatataaatgtattgtagctttccggtgtcagccacggtgtatttttccacttggaatgaaattgtatcaactgtgacattatatgcactagcaataaaatgctaattgtttcatgctgtaaacctcctaccgtatgtgggaatttatttacctgaaataaaatctactagttgttagatggagtgcacatacatttctgaagatggagaaaaacaggtgtgcctgctgatcaggtgctgtgggctgccctgcagtcctggtgagcgacagacactgaggcaggcttgtctcatgaacaggctgcctctgcagtgaaagtttttgtgtattttttttaacccaagctagttttctaatgaataatacttgactcactaatttcccctcctcctccttctcctcagttctcctaacatcctcatgtgatccccagactcaactccagtaatatcaagctttcctattttcccatgtaaaaaaatcccatgactctgggccatgttaatatcaggcttttgtgggaacaggtggcctcaccccataaatcattaaataccattcagcttgaatcattttaatgtgacagtcacaaaccagttgctctaataaaaactctgctaaccatccttctccttagctctctagaacaatctcagttatccctagggatgctccccagcatccagaaaagagaagtgggatcaatcatcctgcctttctccccctcctctcttggagggctgcctgagcccgtggcctccacctcccctgctttgtataatttgaaatgcagatttgtagtgaaggcagagttcacctctgcattgaaagggaaggcaggcccagagcttccttccctgccctctgagatgtgcatttatgtctcaggatggatgagctttggtaggaatgctcaaaaccaggaccagccagacaaactggcagtccctgtaagcggttcccgggtcatagggttagggcacccctgtttaactttggggtggggaaagtatctggttttctttgataaattgcttgtgaaccacatttgccaagtggcctccaggcctcaaactcaaagaccgagctaaatcgactcggaaggcaatgctgaatgaagattgtgggaactgagatagatacactcctctatgttgcaatgtgattaatggttctactaattttatctaagggggcgcagagaagaaaaagtggggaaaaaagaaaagataggaaaaaagaagcgacagaagaagagaaaggctgcccagaaaaggaaaaactagttccccgcttcctgccgatggaccgcagtgcgctctgctctggcgctttgtaactcgctcctccctcttcgggggcagaccccacactccgggcaggtgctcaaacctgacggtaaactcttccctcttcgggggcagaccccataccccggggcgggtgcttaggctttcctgccctggtggccacaccagctgctgtatttatgtgcttcataaggccctgctctgtctgctaaagctatgaagaaagatgtgcagagactggggtggagactaagccaaagaggagctgcctagcctggcagcattgccccgagctgagcccccttggccaggacttcacaaggctcacacctacaatcccatgaaggccagggtggtctgcttagccaggaaagggcaagtgccttcccctcggccacactgccccttgtggccttctcgggacatgtggtaactgacttgctctcaggcccacccgcagcttttccaaatacctgcagccttcagccctgctgccctgcctgtgggagcagctttgactccagtccagaagggtttctgcagactgtgttgggtgagacgcagaaaggatgaaatctcagaacacatgtcagctgcttctcaggaaatcttttctttggacaattcactttagagtctttaaacgggtctctcgtggggaggatagatgtgctctggaactttctgaaggaccagcagcttcagggactcttagtctgtccttccccacttttggtcccaacatccctgggatggtgtgctgtctgggcaccacggtctccatcctcactcctgagagatttctgccttctgtgagttgggttaaagctctggaattatctactatcccaatccactaccctcacctggcaatatttgtctgtttttgtttgtttgtttgtttgtttttgtcttttgccagtttgaattagaaggcaaggctctgattttagtagtgttttggaaaaggacttttttcttcaccttcctctttgcctcatgtgtacacacacacacacatcttgtaccccagacctctgggtataattttcataattggtgcagaaagaagaaatgatctgaagatgtgttaaatggattgcaggggaaggaaggcccagggccctgtgtgtcatgccctcttgggttcctaagttctatgttccttagaggttctagcattaaacagataaagcccttcatggtcctggctgaggaagagtcttgctagggggattcagggaagacccgtgttaccagctcttaccctttatctggacagctctcctaccctgtatcttctcctcagatctgaggatagcaggctggactattggtgggcacctttcaagcccagggctactgtttgtcctgtggcagccggctacagtctcgtctgagtggcctcatctggacccttcctgttattaataaaacgcttctggaggccagatctgtgctcaagccatagttctgcttagaaagggatgccccacccttaccggacactgggaagaactgttggcccctagaaaccaaaggccaaactgaggctgccctgagttggaagaccactttctgaaatgcccatggactctgcctcccaaccattcgtctctcactcctagcagagctgtctgtgcagactgtttcttaggaggcacagcaagctccagggaaccctctgtgcttatgaagctcgtctggtgggcaaccccagcccactggacagagtcctcatggaaatgcctgggaagctgatttcatctaaggatgggttgaagtaggatgtgctcctgcgacttctcaggcaggtgagaggggtagtccttacactgtctagcataaacgccttccggaaggacctgcagctccagagaccacctcctgagcaccaagacctcttctggtggtgtggaaccagccaagagatttcaaggaagagtgattatttgatgaatgctatgggaatggcctcttctcttggagttctgaggcctggggatgcccaggaacactgggcacctgctgctgttagggccaatgcatagtctcagcaccggtgtcctaaggttaaggcggtgcgccttgtcatgtgctccttgtaccatgccatctgtgccagtgtgtgtctgcctcaccctgtgcttgacatgttcacccatcttctctgcttcccgccaccatccagatcctcagcggccgccccggctgtgcccttccctgctctcccgctctctcaggcctcggaaggaagatcggtggctgcgagctgaactaaggagtagggcctgtggctcagcgctaggccacgcacgcagcatcccaggcatgtggtgagaaactgccttaatgtgtctcctctgttcttgtcaacaggaggctcaagatgtgagaggtgtgagtcagacgcccgaggaacttacaggaggagcctaggtctgaagtcagtgttagggaagggcccatagccacttcctctgctcctgagcagggctgaagccgtttgcaagggacttgctttgcacagttttgctgtactttcacattttattatgtagcaagatacatggtgatttttttttttttcatttagcctgattttccaacgtcattggtgacaggccaaggccactatgttatttcctttgttctggtatccttcccttggaggaccttctctgagtagtggctccccaggtttgtcctttgagctgaggcaggaggctcacccattcttctgaataggaactgggtgttcccaccccccaaggactgcagggctttcccaagctgaggcaggaacgtgaggccagggaagagtgagcttcaccctcatcccacgctgtcctcctcaacccaccatgctcatcattctgtctcatccatccatccatccatccattcatcgccatgtgtccgcaagactgtctccatgaccctgaaaaaggactctcgagatgaaatcctttattcaaatgggacagcaagaaggaaaagccaatgtctggtgtctctccccccgcccctaccctgcgcgcatctatgtcttgtttggaatattgtctcttcaaccccctgttcatgtccttctcactcatgatcgatgtcttgtctgtgcactgtctctaacccaaatgcaaaggctgagtgtgaggtgatggccccgaggtccaggttgtagtcatggaaagagccctgctgtctcccttctcagggggcccattttagacacacaaagcccaaagaaaggtggtttgcaacagtgcttagctcgagcctccatatttccataactgttagcttaaaactgtggggttttaccttcctggaaccaaatgcattcttctgttgaggagtaacaggtctcaattcttttcaattaattttaaaagtcaatcactaagagcatcggctttgggccctgatgggcaggcatttccctggaaagggggtgaactacctacctctcctcaagacagccgaagggtgggattggtgccgctctgggaagcgtggccccaggagttttgtcctctgcagtttttaatgcaagttcactgccactttgacaaaagcccaattagaagccagtctctagttccttaaacaaaacagacagagtaaggaaaggaaggagggtggcagccagctggctggacactcgagaaagacggggaagtaagctacagaaagatagtcttcaaaaacaggtgtttgagagtgaatactctgtagaattgttagtggggtgtgtgtggtggtggtggggggatttctacaaaatagtcctttaagttgagtttacagcagatgaaaaatccaaccagcaaaattttgatcaaatttgaacaaaaacccaaaaacctaaaactgttgagcaggttgcgatgaggagcacagggctagctgcagagctggatcctcaggaggatagcgaattattttcaaccctggaatagaaaccacacactggcttgctgtgcaccagccactttgcatctaatccaagctttgaagggtgttgcttgggaggaaacaaatacagccttccatcttcactccagttagggatcctttcaaagtctccttcacagtgaggaaaaagagaagggtagaaactttagggagccggatttgtgtatcaattcctccgctgacagtcagtttctagatggagacagcctgcttaaagcaaatccgaatttaaataggacatttacatcggaaaagtctctccctaccttaatcccccattctcttgctttcaaaatacaagcacagcagtccttgaatggctgttgacccagggcacctagctgtccctgctggtcctggggctgccagaattcccttgggcgccaagcaacctgccaggtagccagtccctctgttacaagcctttgcatctggatagggaaaggggtggagacatacagtctgctttgtgttgaaacccagatttgtaccctgtgtttatacactgctgctggctcccgaggacagtgggactttagcaaggaagtgcagccgaggggtaaagagccctctggttcattgcctgatcggctttgagagagggtttggagggcaaggggctgcattcctctgagggacttggcctgaggcctttcgggcctctccagtgggttctgtttatcctctcatgggtgattatctcagtggtgtcaccaggggcttcctcccagaagtcagtcatccccaggccgtgcacccttttcagctggatgagagccagggatgcattctctccaaacagctaccctggcccattttaaggtaatctcattcttcaaaatgttccatagaatcctccaaattcccccagcagacttctaccctcgccaagttcccaaaacccactcagcaaagttgccaacctcgacgggctagcagtgtctaagcagcgatgggttcagtgttgtgtgtggtgaatactgtattttgtttcagttctgtctcccagataatgtgaaaacggtccaggagaaggcagcttcctatatgcagcgtgtgctttcttattcttatttttaatatatgacagttatttgagaagccatttctactttgaagtcattatcgatgaaagtgatgtatcttcacctaccattttcctaataaagttctgtattcaaatatagctgccaagcatcctcagtgaatgttaccatgtggaattttccacacttggttttaccccctcaaacctgactctgaccgtgcagtcttagcagaagagcttagcaggtcctagtgttcactcttggtctaactgctggtgtcagaagatctctacagggagaggtgttccattttctccacatgacctggattgctccttagaggtcagacagccttgcactgtacaaggcaatggcttagggtaaagtcccaggagttttccctacagtcccaagaatttggaagaggaaggcccacactacacatgcaggtcatggtggaaggtgacagaggaaggactctgtccctgtaagacagctggaaaccacaatattctgcatgttcctatcctgggtgaggacgctaatggaagtcaaaggggaatttgctaactgctgttggccagcttcctccaagaatcctgcttccccaacagacagagcctttgtctcttatagtttggtcttcagattctctttatcccacattcagccatttttgtaaaagagaggctagcaccagctccaaatatccaaatctgcagtgtttgagatctcactgcgcctcctccataccaacacatttgccattacttatagggtagttttcatgtgagttctaagttgattaacacacaagaattagaagggtgggaggctctaggaaaggcactgtgggactatttgactgcatgggtgtgaaaatgtaaggaacaggcaagagcttggatcccattctctctgcccacattgtgacttgagatatactaattgctcttgggggtctcagtcatataccatccataacagagttaaactgagagagatacaggatcagctagaatgaaaagcccaccccatgcttccagaaagtcccctctttatacctcctgtgatatgaactagaggaaaagcaattgactttgcttctcaaacagcctacggcaaagccctgtgagtttg

SEQ ID NO: 2 - DNA, 20 bp, Artificial sequence
  Primer
ccagagccaacgtcaagcat

SEQ ID NO: 3 - DNA, 20 bp, Artificial sequence
  Primer
cagccgtgcaacaatctgaa

SEQ ID NO: 4 - DNA, 29 bp, Artificial sequence
  Probe
tgaaaatcctcaacactccaaactgtgcc

SEQ ID NO: 5 - DNA, 16 bp, Artificial sequence
  Synthetic oligonucleotide
gcatgttctcacatta